SEQUENCE LISTING (30 sequences)

SEQ ID NO: 1 - DNA, 1673 bp, synthetic construct
  misc_feature 1..1673: note = source = /note="Description of Artificial Sequence: Synthetic polynucleotide"
  source 1..1673: mol_type = other DNA
atccaggaagggctatggttttcatcgaagatagacaaatagacagcatgccaatgatgatcagaagaggacgagttttggccatatctggcatgtttttcatgccgattctatctgagttcgccaacctactttttgtaggtcgaggagagcttttagctcatcgaactcttaccagtcatcttatttcccagcaataacgaggttgggttttaggacttgcttcgactaggaacgggagggagaagggaacgagatactcgtagattttgttgaccgaaacaaaccaaaccgcagctacgacgcaccatgatggtatctcgattttagctcagggcactagtggtaggtagtggtggggtggcgaactacctgtctatcttttcctcaagactagaatcctcatcgtgatgagtacaggaacagtaggacagctgattttggattcccagagtgactttttgtcactaggcacctcagcgaaatctatctcggtttttccgagaacagtaccagttttaggctcgcggttcttggaaccttggcagtagacaacctttccagggaacgtgcttttgacctaacttggatgctttttgcatcctgttagtagcggcctccgtgacgtagtttttctacgtagctgatggattttgtaccgctgcagtctgctacatcagggacggactgattcacctctagctcacattttctagcgggtggtgagctttttgctcacgagtggaattcaacggccctttcaatctttttgattgaagcatccgttgttttaattccactcgcagtacatctatgtgctcagtctccgtttttcggagtagtcgcacatagatgtactgccacccgctagtgtgagctagatcatgatcagtccgtccctgatgtaggtttgtgcagcggtactccatcagctagtaacacgcaagtggtacctcctggctttttgccaggctaagccacttgcgtgttactcacggaggccttttgctactaacagcttcgtttttcgaagcaagttaggtcgcacgttccctctcgtggttgtctactgccaaggttcagtcgaccgcgagcctctggtactgttgaaactttttgtttcatagatttcgctttttgaggtgcctacacgatctcatcaacgctaaggccactttttgtggctacctcgttgatgagatcgtgtctgggaatcctcagctgtccttattgtcctgtactcagatgaatgaggattcttcgattgagggatagacaggtctgatgccaccccaccactacctaccttgtctgccctgagcttcgagataccaggtgagtgcgtcgtagctgcggtttgcagactttcggtcaactctacgagtatggaaatcccttctccctcccgttcctcaagaaagcaagtcctcccaacctcgtactgtctgggaaataatcacgctggtaagagtagtctgagctgctctcctcgagtagctcgtcgactctagtcggtgtgtttttcacacctcagtgagtcgacgagctacttggcgaactcttttagatagaatcgctctctttttgagagcagatatggccctcgtcctcttagttccatcattggcatgctgtctatactagtatcttcgatgccatagatgctcctggat

SEQ ID NO: 2 - DNA, 3561 bp, synthetic construct
  misc_feature 1..3561: note = source = /note="Description of Artificial Sequence: Synthetic polynucleotide"
  source 1..3561: mol_type = other DNA
atcacccttggtctcagacggatcaatcgctgtgttactcgtacgggcgattacagattcactgcgacacctgggagatgccgactcccatgaccaacttttttccacgtcatggtcttgttttcaccatacgtcccctgttttttgtcgagacgtttagactcaacgcccggcactcacgacgtgagaatgggcctctacgtcctgctcgtcgtctacacatgtgctacaccgtgtttagttttttggaggcacggtgcagcttttagtacatgtgcccagagtcttttgccgtcgttgtagcagcaatcatgttgtcaagcatgtagcttttacggagagatccgttcttttttcggtcgatctgtttccgtacctgcgctaggccgatgtgatgcctggctcgttactttgcactgtccgacaggttattccgagttcggtttacatgttttttgcctgaaaccgactcgttttagtaacacgaataggcttttttgcgctttcgtcaggaggcagtctatgaactggctttgggacctaccatgcggctcctgaagctgaacgacacggaccttcggtcggtgtagattgtctcggttgtcatccatatggcacggaaagacagcgtgttacccaccgatcgttcgagcagctcaaccggattggaggatcagctcgctggagttctggcgacgcacgacgcaccttttaggacaacaccgattaggccttaaagctacttacggtatctgggcattgtggtctacttccaggtgtaggtccctaatcccggcgtgctacaagcccatgaacagggccttaggcgtccaccttccctttggaagatttccagcgcagcgacacagcgatcccgtggttagtaggtgatctgttgtccgtgagacgtacaaggtattaccactcgacgaaagtgacatatccccagaaatgttctgcatttagtatccctggatgcttgctgtcttccatgtgcaatgtaaaaccgctggagttggctttggtgtcgtcaggtaagcgtttggcagaggcaagagagcaaattcgccataagagagatctcgcgaggtatgagggtaccttgcgctcttagccatggtgcacctcacacttcactctctggtggttcgcagaggcctgagctgttcgcacgtccgctgcacctcgtgaccacacttgttaggaatcgaatgggacgactaatgagcctttgaaactggttgacgtctccagaggacgtgtctaaggctgaggcaatgctggctgacacggacaacctggtcacttgcgctccgtcgtcacgactcttggggccatgaggttgacgagttttttgcctccaacctgcagcttttcaagttaagtgagaccttttttcacagcacttcatcgccaactcgccaatgcgacaagctgtctctggaaaacgcacatacgttggagtggatggagattccgtagacgtatgactgttttttcagtgatacgtgatacttttatgggtaggagaagtggttgagttcgtaccatttgccagtctcgtcttcctccagaccctacgttttcatatgacgtcagtacttttttcgtgtgacgtggtttccccgacactggagtcgctttggcaaggggttgtggcaaatcgttaacggtttgcacgcaacatctgcatcatggcaaccttttttcggttccatgaaagagttttctgagtgaccagtctagtttttctagacgaactactcagctcttgctcttaaccgttttttggttgaagagctgcagttttatgttgcgtgcaaacccgggacgatttgccacaaccccttgccaaagcccaaccagtgtcgggttttgaaacctgccaacacgttttttgtacttggcacatatgcgtagggtctgactcaagacgagactggcaaatggtacgaactcaacgaggagtcctacccatgtatcccctcacactgttttttcagtctgagggctacgttttgaatctccatacgctgcaacgtatgtgcgttttccagagacacgagaccgcattggcgagttgttttgcgatggcccactgtgttttttggtcttgggcaacttggctgcgaaagcgaggcttttttctcgtgctttccatggttttccccaagagttcgttcgacggagcgcaccgcaccaggttgtccgtgtccagtagcattgcctcttttagccttagacgtcggatctggagacgtcaaccagtttcaaagacatcgtagtcgtcccattcgttttattcctaacagccagagtcacgaggtgcagctcctccgcgaacagctcgctgctctgcgaaccttttaccagagagtctcctctgaggtgcaccatggctaagagcgcaaggtacgagtatacctcgcgattttgatctctcttccctgtaatttgctctcttgcacgccgcaaacgcttacctgaacctggcaaagttttccaactccagacgacgtacattgcacatggaagacagcaagcatccagagtgactaaatgcagttttaacatttctggggatacgcaactttcgtcgagtggtaataccttgtaccacacacggacaacattttgatcacctactaaccagttaatcgctgtgtcgctgcgctggaaatcttgactagggaaggtggttttacgcctaaggatggcgtcatgggcttgtagcctctgcggattagggacctaccgacacaagtattttgaccacaatgtgcgattaccgtaagtagctttaaggcctaatcggtgacagcctaaaaggtgcttttgtcgtgcgtcagtgtgactccagcgagctgaggacgtaatccggttgaaggcctcgaacgatcttttggtgggtaacccactctctttccgtgccatatggatgacaacgcttgtaatctacaccgaccgttttaaggtccgtgcgtgacagcttcaggagagtgatggtaggtcccaaagcagcctcatagactgcttttctcctggagccagcgcttttttgcctaggctcgttactcgagtaatacccaggcttttttcatgtggtattaactcttttggaataacctacgtcccagtgcaaagtaacgagccaggcatcgctcatgcctagcgcaggtacttttggaaacttagtgaccgttttttgaacgactaactccgtgctacatgcttgctgtcatgattgctgctacaacgacggcaaaagacatcgcacacatgtactgctgcacacctcctccttttttctaaaaggtgtgtagcttttacatgtgtagcggtttagcaggacgtagaggcccattctcacgtcgtgggatccgggcgttgattttgtctaactccgtcgacttttttcagggcggagatggtgcaagaatccctcgtggttttttgttggagggatgagtcttttggcatctcccaggtgttgtcgtgaatctgtaatcgcccgtacgagtaagtctgcgattgatccttttgtctgaagttcagggtgat

SEQ ID NO: 3 - DNA, 3561 bp, synthetic construct
  misc_feature 1..3561: note = source = /note="Description of Artificial Sequence: Synthetic polynucleotide"
  source 1..3561: mol_type = other DNA
atcacccttggtctcagaccaactcgccaatgcgacaagctgtctctggaaaacgcacatacgttggagtggatggagattcgactcccatgaccaacttttttccacgtcatggtcttgttttcaccatacgtcccctgttttttgtcgagacgtttagacgctacatgcttgctgtcatgattgctgctacaacgacggcaaaagacatcgcacacatgtactgctacaccgtgtttagttttttggaggcacggtgcagcttttagaactgaagcggtttagcaggacgtagaggcccattctcacgtcgtgggatccgggcgttgattttacggagagatccgttcttttttcggtcgatctgtttccgtacctgcgcttggccgatgtgatgcctggctcgttactttgcactctccgacaggttattccgagttcggtttacatgttttttgcctgaaaccgactcgttttagtaacacgaataggcttttttgcgctttcgtcaggagtacttccaggtgtaggtccctaatcccggcgtgctacaagcccatgaacagggccttaggcgttgttgtccgtgagacgtacaaggtattaccactcgacgaaagtgacatatccccagaaatgttgatcgttcgagcagctcaaccggattggaggatcagctcgctggagttctggcgacgcacgacctgcatttagtatccctggatgcttgctgtcttccatgtgcaatgtaaaaccgctggagttgggcagtctatgaactggctttgggacctaccatgcggctcctgaagctgaacgacacggacctttcgcgaggtatgagggtaccttgcgctcttagccatggtgcacctcacacttcactctcagctgaggcggtgtagattgtctcggttgtcatccatatggcacggaaagacagcgtgttacccaccgcaccttttaggacaacaccgattaggccttaaagctacttacggtatctgggcattgtggtcgaggcaatgctggctgacacggacaacctggtcacttgcgctccgtcgtcacgactcttggggccaccttccctttggaagatttccagcgcagcgacacagcgatcccgtggttagtaggtgatcggttcgcagaggcctgagctgttcgcacgtccgctgcacctcgtgaccacacttgttaggaatcgaatgggacgactaatgagcctttgaaactggttgacgtctccagaggacgtgtctaaggctctttggtgtcgtcaggtaagcgtttggcagaggcaagagagcaaattcgccataagagagatcccatgaggttgacgagttttttgcctccaacctgcagcttttcaagttaagtgagaccttttttcacagcacttcatcgcggatcaatcgctgtgttactcgtacgggcgattacagattcactgcgacacctgggagatgcccgtagacgtatgactgttttttcagtgatacgtgatacttttatgttgcgtgcaaacccgggacgatttgccacaaccccttgccaaagcccaaccagtgtcgggttttcatatgacgtcagtacttttttcgtgtgacgtggtttccgtagggtctgactcaagacgagactggcaaatggtacgaactcaacgaggagtcctacccatctgcatcatggcaaccttttttcggttccatgaaagagttttctgagtgaccagtctagtttttctagacgaactactcagctcttgctcttaaccgttttttggttgaagagctgcagttttatgggtaggagaagtggttgagttcgtaccatttgccagtctcgtcttcctccagaccctacgttttgaaacctgccaacacgttttttgtacttggcacatatgcccgacactggagtcgctttggcaaggggttgtggcaaatcgttaacggtttgcacgcaacatgtatcccctcacactgttttttcagtctgagggctacgttttggcatctcccaggtgttgtcgtgaatctgtaatcgcccgtacgagtaagtctgcgattgatccttttgcgatggcccactgtgttttttggtcttgggcaacttggctgcgaaagcgaggcttttttctcgtgctttccatggttttgatctctcttccctgtaatttgctctcttgcacgccgcaaacgcttacctgaacctggcaaagttttagccttagacacgtccgagtgagacgtcaaccagtttcaaaggctcatgccacgtcccattcgttttattcctaacagccagagtcacgaggtgcagctcctccgcgaacagctcgctgctctgcgaaccttttgatcacctactaaccagttaatcgctgtgtcgctgcgctggaaatcttgactagggaaggtggttttccccaagagttcgttcgacggagcgcaccgcaccaggttgtccgtgtccagtagcattgcctcttttgaccacaatgtgcgattaccgtaagtagctttaaggcctaatcggtgacagcctaaaaggtgcttttggtgggtaacccactctctttccgtgccatatggatgacaacgcttgtaatctacaccgcctcttttagctgagagtctcctctgaggtgcaccatggctaagagcgcaaggtacgagtatacctcgcgattttaaggtccgtgcgtgacagcttcaggagagtgatggtaggtcccaaagcagcctcatagactgcttttccaactccagacgacgtacattgcacatggaagacagcaagcatccagagtgactaaatgcagttttgtcgtgcgtcagtgtgactccagcgagctgaggacgtaatccggttgaaggcctcgaacgatcttttaacatttctggggatacgcaactttcgtcgagtggtaataccttgtaccacacacggacaacattttacgcctaaggatggcgtcatgggcttgtagcctctgcggattagggacctaccgacacaagtattttctcctggagccagcgcttttttgcctaggctcgttactcgagtaatacccaggcttttttcatgtggtattaactcttttggaataacctgtcggatctggcaaagtaacgagccaggcatcacatcgtagtagcgcaggtacttttggaaacttagtgaccgttttttgaacgactaactccgttcaacgcccggcactcacgacgtgagaatgggcctctacgtcctgctcgtcgtcttcagttctgctgcacacctcctccttttttctaaaaggtgtgtagcttttagtacatgtgcccagagtcttttgccgtcgttgtagcagcaatcatgttgtcaagcatgtagcttttgtctaactccgtcgacttttttcagggcggagatggtgcaagaatccctcgtggttttttgttggagggatgagtcttttgaatctccatacgctgcaacgtatgtgcgttttccagagacacgagaccgcattggcgagttgttttgtctgaagttcagggtgat

SEQ ID NO: 4 - DNA, 6552 bp, synthetic construct
  misc_feature 1..6552: note = source = /note="Description of Artificial Sequence: Synthetic polynucleotide"
  source 1..6552: mol_type = other DNA
caactcctcgattcccgcttgtttgcacttgtatgtacatagtgtcagatcgcttacgcttgcgtggcgatcatctagtcgtcgttttttgttccactagatagtattttcaggcgttgactaggcgcccgaggtatttcaagaagacaactgattaagtgtgttttcaagtccaactactacttttttgcggaagttgtcccttctctaccgtcttgctcagtgttcagagactttgctggtgaagtgccgcaccgcctgctgttaagagagcttttttgggagcttaacgcaaattttccacgtaaactgttggttttttgactcagtttaggactgagtcaagcatggtcggtcccacagaatccggaagcccagtcagaaaacatggtagcgcttcccctgggttttttcagaagggaagcgccattttgcatatttaccactcgtgactcgtagggaggacgcctagtgaagcgcgtctcattttctcgaggagtgagggcttttttccaggcactctgtgtcgtcccgttaaggatgtgagtgtcatggcgagcaccagacagaggccgtgaacgcttgaagcgagagccgttttttgacatctcgctggtgcttttcacctagcaactatcgttttttcatgagttgcctctgcttgagcatcaagtcgtaatcgtccatgttaggtgcgcttctagaagaccggtgtccagtatgggtgtgatctcctgtcttgcctggtaccgctttccaggatcgggtacgggactcgtgcccggaacatcagggcaaatcgcggtctcatataggactgtacccacacacttgtcttcgccgttcggaactttcggagtatacacagtcttgttgcgctttaacagatcatctgtcataagtaaggggttgcacatatccaagatgggattgcaacggtcaaccggatattacgtattttcctgggctagcgtcggatttcggcttccttgggactgaacagggatcgtttggtgctaggcgctggcctttcacacgggcgtgtgcagcacagatcatctaatcatatgtaacttgaccccgtttgcctcatcagtcctcatgatgagtaagtagccatcttcagtaaatcttcgtctaacatagggtacatactcatcatggacactttcttccgaatgcctgctacccgcacctcaaccgccctaagactattggtgcttcttcacttcgtctgagtcagcctctccatctgtcactccaagggatagcggacgaccccgagtgtcttgaattgcatctacgaaaacgttcggactacttttctacgtctgttatctcagtccttgccgacgtcgtaggtcgtgataacgtccaagtcgggttgcgaccaaggccagacggaaggtggattagttgttacttcgccagtgagagtttgcctagcttggatcactgacgtctgatgttagcgtaatctagatcactggggttctgccacagtccggtgagtacagcacagatcgtatacatcacgaacgtttgtccagtcgcggaacactgaaatgaactaacgctgacatgtatgacaaccaacatgattacacacgcttgtgagcgagtctctgcatgaggagtgctccacagtgagtgactacgctgccagctgcagcgcgcgtttgtttcgggtcccttcagcatcgaactgatccgctaaacgtcttcacaggcaagacgtcgcgaatagcggaacgatgcaggtatcttagtcatgcgcaaccaaccaccacaggtgtgctactacgattcgtccgtcggcatttaacgctccgccgttggatcgataaatggtttctggaattatatagccggtagatcggccgagacacttacgtataatgagcccacttatcctcttcttgtgaacactgcttgctatactcttgcacacctcggtcgcagacggtaaagactactgaatacggcgatagcgtatgtgcgcatgacgcgcggtgaacacatggtgccaaacactttcttcgtgacccgaattgaataatcgcttatacaacctgaaagtgccgtacgcattcgccgtgggctaaagcgcgatcaagtctcacccttggtagtcgagttgtcgggcgtagcagctctctgtcctcgtcccattactttgcttagtgaatggaagactgtggtaatgctcctcacgaggatagtcaactgaccgtcatgcagctgacgacgtttaacgatcctgcatctctcccaagatagtcagatccgtaacgagacttgcaggactcgaataccctctaactatccgcactagagtgccggaggcagttcgcccttgtaggatcggaggtagtcagttgatagaagggctgatggctcgtcctcgaagttgaactccctcagccacacttggttcctacagcagttgcatctgaagaatctgtcgacacagacatgccttcggcgctcgtcccacttatgcaagcgcattcgtcgaggcaaggagctcacttagagcgtctgatggtttagtactgagtgagatatcgtcccttctaatggactttggtcctgcggttattttgggagcggcacatgacgcgccaagcactcatacttttttcgatcagtgctcgtacttttcttatgcttcgagccacgttgccgcgcatatcaattctacctaggcatacctgttttcttaacagccactgtgttttttgcggctggctggtcagggcgactctgagaatagaaaggtgcgtgtaatcatagagtgagagcgggtgtctgtggtctccgatatcttttttgagcgcggagagtttattttatgcctcgtggggctcttttttgtagcccacgtcagattgctgacagatcacgttgatttcgttccagtaaagtgaaatgaactctgaccgggagtgccaacgatacttttttgtaatgttggcgcatattttaagcgcgtgactgcatatcagatgagatgttagggaggtgagaagtactcaccttttggcgtgcacgccgtggttttttgattagcgtggtcgggtggccggaatggtatgcctaaacaccagacatctgaactgtctggttcataatgtgacacttgtctggcttttttcgtgaacaagtttgtattttactagggaggatgggctttttgcccacggatcctagttacaatgatcgtcacgttttttgccagcgatcagtcacttttattatgaaccgtaatcttcagatgtctggtgtttaggagcgtcattccggccattttcccgacggacttaatcttttttccacgagtcccacgccggtgagtacttaagctctccctaacatctcatctgattgtccatcacgcgctttatgcagtatgattacttttttgtatccatactactccttttcggtcagagtagcccacactttactggaacgaaatcatgaccatctgtcagcattttatctgaaagttgctacttttttgagccaacttaggcattaaacgaccgtcgctcttttttgatatacggtcccacattttgacacccgcttgtgcactatgattacacgcacctttcatctgtcagagtcgccttttctgacctaggcgccgcttttttcacaggcctagttaagcaggtatgcctgacgtgaattgatatgcgcggcaacgactgtagaagcataaggtacgtctatcgatcgttttttgtatggatagatggcgttttcgtcattcttcgctcccaaaacctaaccaggaccaaaacgacttagaagggacttttgatatctcacctctgtctaaaccatcagacgctctaaacatcctccttgcctcttttgacgaatgcgacgaagtaagtgggacgagcgccgaagagggctctgtgtcgacttttagattccctggatgcaactgccaggcaaaccaagtgttcacaagggagttcaattttcttcgaggactacagttcagcccttctatcaactgacacgtcccgatcctacattttagggcgtgtggcctccggcacagttccgcggatagttgacaagtattcgagtcttttctgcaacagccgttacggatcaacgattcttgggagatcccaaggatcgttaattttacgtcgtcagtggacagacggtcagttgactatcctcagcttgagcattaccattttcagtcttccagactggaagcaaagtaatgggacgagggatctgagctgctacgttttcccgacgtcacgactaccaagattagaacttgatcgctgcacagcccacggcgttttaatgcgtacgtgagactcaggttgtataagcgattatgggcatcgggtcacgattttagaaagcaggtggcaccatgtaagtgtcgcgcgtcattatgtcatacgctatcttttgccgtaactggtagtctttacgtagaacgaccgaggtaacgtagagtatagcattttagcagtgttctgactgagaggataagtgggctcattacgaccaagtgtctcggttttccgatctaccacttcaataattccagaaaccatttatgagcacaacggcggagttttcgttaagcctcgacggacgaacgactttagcacacctacgacggttggttgcgttttcatgactaagtactgagcatcgttccgctattcgcgagaggatgcctgtgaagttttacgtttctgtgatcagttcgaagtgatagggacccgactaacacgcgcgctgcttttagctggcagccgtaagactcactgtggagcactcctccaagtgagactcgctcttttacaagcgtgtagacagatgttggttgtcatacatgtccatactagttcatttcttttagtgttccgcttcactacaaacgttcgtgatgtatacacagagtgctgtactcttttaccggaagcgggcagaacccctgctgactagattacgaacaaatcagacgtcattttgtgatccaaggtcctaaaactctcactggcgaagtaatggtcaatccaccttcttttcgtctgatgctggtcgcaacctcgtagggacgttatcgtggtctacgacgtcgttttgcaaggttcaagataacagaccgtctgaagtagtccggtgcatttcgtagatgttttcaattcaagaatacgtgggtcgtccgctatcccttggcaacgcagatggagagttttgctgactcagcttgcatgaagaagcaccaatagtcttgcatgggttgaggtgcttttgggtagtgttcattcggaagagttcacccatgatgaggcgcaaccctatgttattttgacgaagattatacctagatggctacttactcatcatcgtctctgatgaggcattttaacgggaactagttacatatgggtgagtgatctgtgcgctttacgcccgtgtgttttaaaggcgtctgcctagcaccatgactaccctgttcaggatgcaggaagccgaattttatccgacgcttcatttggaaaatacgtaatatccggtacgtggttgcaatcccttttatcttggatactcactaccccttacttatgacagatgtattcttaaagcgcaattttcaagacaacttatactccgaatctagtgaacggcgaaagagggtgtgtgggtattttcagtcctatagcacttcgcgatttgccctgatgttcctcaatcgagtcccgtattttcccgatttcagaaagcggtactgtaggagacaggagaggctgcccatactggattttcaccgggtgcctagaagcgcataaccgatggacgattgtccattgatgctcaattttgcagagtttcttcatgttttttcgataagaaataggtggcaccgtggtcatgtcttttttcggctgaccactcaagttttcgttcacggctcagtactggtgctcgccatgacactcgtgagcttaacgggacttttgacacatccgtcctggttttttgccctacggactcgagtgagacgcgctcgttgaggcgtcctccctacgagtcaacgtatgtaaatatgctggcgaacaagttctgttttttcccagcttgttcgctattttccatgttttcacaagaggcttccggattctgtgggactacgtatgcttgactcttttagtcctgaggagagtcttttttccaactcctcacgtggtttgctacatcctcccttttttgctctgatgtaagcagttttgcggtgcggcggctatccagcaaagtctctgaacactcgatcagacggtagagttttaagggagcctatccgcttttttgtagttaggcgacttgcacacttaatcgaccatcttcttgaaatacctcgggctaggactcaacgcctgtactactgcagggaacttttttcgacgctgcagtgatcttttgccacgcaaggtagtccgatctgacactatgtacataatgcagcaaacaagcgttttggaatcatccggttgg

SEQ ID NO: 5 - DNA, 7521 bp, synthetic construct
  misc_feature 1..7521: note = source = /note="Description of Artificial Sequence: Synthetic polynucleotide"
  source 1..7521: mol_type = other DNA
atcacgacgctgtttcacggttaactcctcacgctccacagaccagtacttccgacttttcaaccttgtaccactaggagtgtttcctcgagttaaccttttttctccaactcgatggtattttaagattggtgtctggcacatagctgctcctagtaccaaaccttagattcctacgttctataggttttcagaggccaagatcggttttttgggagcttggcaccacgctcatgtgtcgggatcataggctgacgattactcggtacttgcgatcatgacatagagattgttgtgattcgctgtccttttttgttgggcgaatacaggttttataccactgacgaaggttttttcgtgtgtcaggcataggtcgttcgtagggtcccctatgcgttgatagaatttggtgttcggaacgtcagaacaattcgggagcagcgtcatggacttttttcgaagtgacgcatgacttttgcgtactgttccctcgcaaatatgccaggagtgtgaacacatcctggtcaccctcagggaccattttacgatgtcctcaattcttttttgtcccgaggagctccccttgctcaatggtgacgaaagtcataccgtccagagccgtgtggatggctacggtcagaggagggatctctgtcctggtatgggtggaagtcctcgtgagagttgggtgcgcgtacattgccagacttcggcaagtttagccttaactcgtcaggtgccagacatcctctattgtccgacaactgtagtctcaaaggcgtctggctgcacggcttattacgacgactccgtcaatggactgccttgacagtcggtggcatagcggtgttatgagtcagcgtgaggttactacgatactacgagggacagatgttctctgtacatgtgcaagtgggtgccatttaagctagtagcagagtgcgatgtgcactgtggacttcttcatgcttggacttgtaaaccgagagctcacgacaccattcaatcggcgacagagcacaccgtcgaatgcataggaccttggcttttgtatcctacggtgcataaatgccagagatgatgtagctctccgatatgcgatgtgacaccgtaccagcacgtacgtatcccatgtacggaccttgatcgcgcgcataaccgtccgcccactatgtcggctgctgctactccctcgattatgtaccctaaaaggcacgtagcgtgaaggggcatcatttggtccacaatgtgctaatgtttgaagccatagcagggcgggtgtatgtatgccgtggtgaaagcgtcacgctgggtagcagcgatggccaccaagcagcgtgcatcaggtccagaatagcctcgcaaagccagaatagacacaccaagtccatgtccagtctcgcaaaagtagtgaaggcttttgctgggtttcgaccttagttactggcagcatggatagcacatgacgccaacgtgtgaaagtaccaatgtcgactgcgcgagagttagactgaacggcagtacgtgctatctcctgcagcggtgattcaatctcggaggagtaggcagagcgtcgaggaatagtcactggcgataatcttgtattcgagcgctgccgcatgcgaattagtcaccgtgggtgtatctacgttggctgcagctcgcacgtcggctgactccgtaccgctcttccgaactatcgaccataacctgccggcgaactcctttgctaggctccggaagcatagacaatagcttcgagtacccatggccttgtagctgaaagatgacttgccacaattggaggctcgttccaatctgtctgcgattcaaacctcttcccaaatctatacggttacctcgcagggctcctcatttccatgattcattctacccgctaaccgtccttgcctggaaataacctgtccgtagcaggtagaccgcttgccatcgtcagcgtcctccgtgcggtaaacttgtcactgagggttacctttggtcaagtttctgaccgatcagtgacacccagacctttccacaccccgaaggctaggcaagactgtggtatggaagtgtgggtgtccctgcctacgcctcatgtggacgcactcattgaccgactcgtggtgctctacatgttccacagcaacggaaggggactcgatggtgcgtgctagagagttcatgtacggaatccatgtctatcctaggtcgattaggcgttgacggttgctcgggagtcctcaagttctatctatatcggggcatctgagatacggtgtctatccatatactccggtaccgattaacaggttcgatatgccctcaagactgggttgtacagagcaggctggtccatctaggctagttattgcgtccggatggctgcaggccacgcatgcctacgcttagtatgcctgcagtctaaatgttttgtccggtcagtgacacttgacgaagaatgagtggattgtgctattaacacgtcggctacctcacattctgtctaccctttactgcctcgagctccgagccggaacgactacacagtttctgaaaacttcgaaactccatttgaaactgcgagacgcttgcacgtctacgcatggctgttcgtaacggagtgaccgtcacactacagggatacagcaagccattcgcgttgccaacagcgtctaacatgtattagaggagtcggatttcttatgtgtcgcgtgcggactctcgatctcgttccactctctggaaaacatttgtcttaggggtgttgacccgttcggttgtcattgaaatgacccgactcgcactaactgcgtattggtttctccaaaatgttggggtcactccaaggtgacgtctcgtgccgtgttgcgcaccatttccttcacgttagtaccactttctctggggtagctgagggtacaataggttttttgtagctgtaccatacgttttcatgactgtgaatgtcttttttcctgatcacatgacggtgctgaaccgagccttcatcaacgtcctgagtactgcctacaacaagttctggtctgctggattacctgggagagtaggttttttgccactctccctcgacttttgttatcttgactaggaagcgaacgcctgggttggtcgtcgatagcaccacgctaccagacctcttttgaggtagctcctacccttttttgaactggagccaattgcaacatgcttccgactcatggtcatcattgatgggtcatcaatacgtgttggacagtagatgcgtacctgcagcacgagttttttgcaaggctgcagacacttttattcccatctcaggacttttttgtaccgagataattgtctcattggtgccaccatcaccgtccaccaatggcccagatgcgattccaaagctgtgctgtccgactattgtcgtacacttttttatactcgacaaggcagttttcgaacggaactcagggtcggaaccattatccgtgcggatacacgcaatgtgacactaccaggattttcctctacctagcctagttttttcccttctaggtgacacggcatgtgactgaggcaaaagcggtttctgcgatggctaatttcgtcgaagccgatcaactattttccagagaggtaccttttttgtgagctctctcttgcttttgagactacagcctctagtttttctagagatcccagtctcgcaagcatctcctcacttttttggtacgagatgggaaattttatagttgatcggcttcactcaaattagccatcgcagaaaccgcttttgagcaagtcacatgccttttgtgtcaagccaaagggttttttctaggtggcttagaggtcctggtagtgcgtgattgcgtgtatccgcacggataatggttgtgcccctgagttccgttcgctgcccaggatagtatttttttgtgtaatcctgtagtcttttggacagcacacgttcagaatcgcatctgggccattggtggacggtgattcacgcaccaatgagttttacaattgacgtggtacttttttgtcctacgtcgggaatgtgtccgtctacttgcttttttctcgttagacgggtacttttgcatctactgcatgctacgtattgatgacccatcaatgatgaccatgacttcgaagcatgttgttttcaattgagcagagttcttttttgggtactgcttacctcgaggtctggtagtcgggtgctatcgacgaccaacccaggcgttcgctgtagcatcaagataacgtcgacatctggtggcttttttcctaccagatgaggtattttatccagcagagtacgccttgttgtaggcagtactcaggacgttgatgaccagtcggttcagcattttccgtcactcactcaggttttttgacatgtgaggtcatgcgtatatactggctacttttttcctatcagtatctcagttttctaccctgacgaaagtggtactaacgtctctgaaatggtgcgcaacactcaccgagacgtcacttttcttggacaagccccaacattttggagatcgtaatacgcagttagtgcgccaggggtcatttcattttatgacaaccggcttcatcaacacccctaagacaaatgttttcatgcccgtggaacgagatcgattttgagtcccagtgcgacacataagaaatcactatcctctaatacatgttattgcctgttggcaacttttgcgaatggctctgacgatccctgtagtgtgacggtcactccgttacgagaccccatgcgtagattttcgtgcaagcggtcatgagtttcaaatggagtttcgaagttttcagaaatcccgtagtcgttccttttggctcgtctgtcgaggcagtaaagggttcgtagaatgtgaggtagccggtccgttaatagcacttttaatccagggtttcttcgtcaagtgtcagcagccggacaaaacatttagggaccaggcatactattttagcgtaggcaacacccgcctgcagccatccggacgcaataacgctcgaagatggaccagcctgttttctctgtacaatggccacttgagggcatcagcaacctgttaatcggtacgcatgtatatggatattttgacaccgtattcggactgccccgatatagatagaacttgagggttaaggagcaaccgtcaacgttttcctaatcgactgctactagacatggattccgtacatgaactctctagccgacaccatcgagtcttttcccttccgttatggacgaacatgtagagcactggctttcggtcaatgatctggtccacatgagttttgcgtaggcagtcctcgccacacttccataccctccgattgcctagcctcgctggtgtggaaagttttgtctgggtgtcactgagcactcagaaacttgaccaaaggtaaccctcacacgcaagtttaccgttttcacggaggactggtcggatggcaagcggtctggtacgtacggacaggttattcgagctcaaggttttacggttagcgccgtacatgaatcatggaaatgaggagccctggtcacaaaccgtatagatttgttttggaagaggtttgaatcaggtacagattggaacgagcctccaattgtgggcgttcatctttcagttttctacaaggccatgggtgacggaagctattgtctatgcttccggagcctcctcaaggagttcgcttttcggcaggttagctgacatagttcggaagagcacctgcgagtcagccgacgtgtccagggcagcttttcaacgtagattgcgtgacggtgactaattcgcatgcggcagctagcctatacaagattatcgcttttcagtgactatggacacacgctctgcctactcacagtcgattgaatcactcgggcaggagatagttttcacgtactgcgctttggtctaactctcgcgcagtcgacattggtacttggtgacgttggcgtcttttatgtgctatctccaacgccagtaactaaggtcgaaacccagcaaaagcgtcgactacttttgcttttgagactggacgctgtgttggtgtgtctattccacgagtgcgaggctatgtgcgacctgatgcattttcgctgcttggcccagttcgctgctaccatcggtgacgctttcaccacgcggaacatacacccgttttccctgctatgaacgggaacattagcacattgtggaccaaatgcagagacttcacgctacgtgcttttcttttactcaacataatcgagggagtactgacagccgacatagtgggcactgggttatgcgcgttttcgatcaaggtggtagaatgggatacgtacgtgctggtacggtcgaggttcgcatatcggagagttttctacatcatcgatcagatttatgcaccgtaggatacaaaagccaaggtgagctgcattcgacgttttgtgtgcgagctcgccgattgaatggtgagacgagctctcggtttacaaacgtaagcatgaagattttagtccagcacgcacatcgcactctgctcgacgcttaaatggcacccacgacgacatgtacagattttgaacatctgttgtcgatagtatcgtagtaacctcacgctgactcataagttggctatgccaccttttgactgtgtgagcagtccattgacggagaacccgtaataagccgtgcagagtcacgcctttgagttttactacagttgctcagaaatagaggatgtctggcacctgacgaactcccgctaaacttgccgaattttgtctggcaatccagaagcacccaactctcacgaggacttccacccataaggcgacagagatccttttctcctccagacgtagccatccacacgggaagggacggtatgactttcgggcacattgagcaagttttgggagcagcgtgggacttttttgaattacgctcatcgttggtccctgagcaacaccaggatgtgttcacactcctggcatatttgtcgacaaacagtacgcgtcatcacatgcttcgttttttgtccacatgtgtgctcttttccgaattgtttgctgtttccgaacaccaaattctatcaacgcatagggacagctacgaacgacttttctatgcagtcgacacgttttttccttccgacttggtatcctgttcgtcaccaacttttttggacatgacgacacaattttcaatctctattctcgcatcgcaagtaccgagtaatcgtcagcctatgactgtgacacatgagcttttgtggtggtctcctcccttttttccgatgagaccctctgcctatagaacggctcaatctaaggtttggtactaggagcagctatgtctgatccaccaatctttaccacaccagtggagttttttggttactggtgggaaattttcactcctagtggtacagcagtgaaaagtcggaagtactggtctgtggacaaggaggagttaacttttcgtgaagggatgtcgtgat

SEQ ID NO: 6 - DNA, 7051 bp, synthetic construct
  misc_feature 1..7051: note = source = /note="Description of Artificial Sequence: Synthetic polynucleotide"
  source 1..7051: mol_type = other DNA
atccagggagtggcgtaccgacacaagagtacgcccctaatgcatggagcctcgtctaggatattttcttccccaggcctattcacagttttttggtctcataggcagctagcactgctagtgggatgagctggacacacttgtggtgaggcagtgcgtcaacctaggcttacgggactacctacctccctgggaggctgtcttcacggatgctgctttggtctaagccttttttcctgtcgaccaatgaatcgcacttggccatgctttctgcatttgggctaggataaggaccatgctcgtgcacgtacagagaagactcttttttgtccattctcttagtccggtatgagcttatggacgaagccctaggatctgtaaggagcatcgagtaaatcgttcgtgtgtggcatagcgacgtcaagcaggtgtacctgttcgagttttcctggaagagttcccgttttttggaccactcttatggcgcaaccatcgaaatggggctgtaatcgcctggatggagcgattccctttaggaccgtccactgcagtggacttttttcgagtctgcagtccgacaaaggcaggtctgttggtcttcggtatgagcatccgcaaccacatgtgcccagaaagtaggatcacttctctgcatgtacgtctcagccaagaggaagaagccagagacactcatgtcttttttgtatgcagtgtcaacgacaagcggttcattgggattgagtgtctatgattcgttcatctaggctcgaccgagagtaagccagatgccttttttcttaggtggctttgtcgcaaatcacggaaccaaaggatgcatgattagcggagactctcttccgatttgcaatcggaggttctgatcgtaccacatgccgacttacaaccatggtaacatgtttctcgccaggtagatcttcgtacttatcccgatagagttcttcgtttgagcctctcatgagttctacactcttgtcacttcggtacagaagtcgtggatacctgattggttaagatcttccacatggcacgacaacagaggcttgcgtgcctggaatcgttagtgatattcctacattgccccattcctccgctaagggtacggtacaccagtaggcatgtcaactcacacacactggagtttggaacaacgagaaattgtaggcagccatggtaacgtttataatgtaacatttcggaggaacagacagctcatcgccttcacacggtaccatgtcctcaatcctggggacatcctgacatgctaggataaatcgggctctacgacgaacttggcctaggtttataacgcgtctatatatctcggaaaatgagatacctaggggaccctacactttctctacaccaagcatctggccctacatgagtttcacatgggttggaatctaatggttggcaatcgcagaacgtgcctttaacctgggagtaacagacctacgattagccattatcccctaatctgccaaccatttgacacgctagggatagatgcttagcccgaatcttgcacttgtgtttccatatggcggatcttcgaaactcatggccacctttcccaacgcatttggatctttgtgcaactcgaagggacaaaggttcctatcaatcagtgtaaaccggtcgctcatttccgcctggtgctcgtgcgttgcttacctaatacactctaaggaatttggaacaagctggtgacggacggctgaggtcgtcctgtttcgggtaactgcacactagcgtcgtatcgtagggtaagtggctcaggggacgcatttgctgacggtcagaatatcgcgtttaatccagttgtctcgacgatttgcaggaccttaatacgctgcatgtcaacggctacgttgatagtgcttcttacgtttcactctccagtagctacagctcgtgaatccccaacactgagcatttaacgcagaccgttagatgtactcacacagtccaacgcttcactagaaaacaattttctaggtaacgcgcgagactctaatcttgtccacacgatcggattaatgccggaactgctgatactagccgtccctctcgatgtgtgacttcggacgatccttattggatgctatggactccgactgcgtaggatagaagataccggctacatccagagccatatgaagtgtccgttgtgtcggttacggtgtcctaacggtcttgggacccgtatcctgcttgcaacacagcttctttcaaggttgctcgtttgagtggtctccccagggttatcaagacagtggccatactagcgttccactcggtaaaatccgacctcggacactcagcctgagacgcagagagaactaggtcggctgaagcctttggtgagtaacgacccttactcggagcgtgtgttcgaaggaaagtcattgacagaaggcacgttttctcaggacctagtgttaggttacgggctatcacatgaactggcttattgaatctttcaccatacaacagatgtgtatgtgcatcctatctaaagcactgtttgtccaagatgaggatctagtatcggttcctcctcacacactgggacacttctgtttgacccagactggtccgaatgctacatgtaagctcttcatagtcttttccagcggcactactgtaggagcccagagtcacaaccgccacatggacacccgctgttgtactccacgattggcatggtaagtgaagttctaggtcagagggtcctattcacgtgcgatactaagtgatacgatagccatcgtgacttgtttgtgactaccaaccaccacgtcctactcttgaggaaaggatcactttcctaccgtagcgatctactacccagaccacaccgaaggcaagatcccttcttgtttgattggctagccatactacgtgaagttgtgcgaaagtgggacgtctacctaccgtctcatattgtatcataaccgagcctgacgttctgtggtagacacttcgaaaccttgccttttttggttgtgtttctcgtctgtcgaccatctacgaagacttcgtcgcatgctgttcaagatgaaagtcggaacggacaaccacagtgtccttttttctgtgatgtggtagtacgtagctgcagctgtcaactatcggattctaagcgcgagtgtgctgctgaggtagacgttgacaccacccgccacaccatagtctctggatgaatgaccccgtgggaagtgtgacgtggttttttctcttgcacactagccgtatagcccaaatcctacagtctttgtctggctatatgccaattgcgacgaatgctatcacaacatggatgttttttgagtggtgttggatgcgtgaacatttctacacacgtagagaaggacgtgcacggagcagtcttgcactgggcacatgtgtgtagtcttgctatccaacacgggatgacttgtgctgcatcgtggcactcgtctagtttttctagactccacccacgatgcagtttcacaagagccaccgtgttggatagcaagactaagtgtgtgtgctttccagtgcaagtgatggccgtgcacgtccttctctacggaccaagaaatgttcatttcgcatcatgacccactcttttttcatccagtcattgatagcattctttgtcgcacgacacatatagccagacaaagactgaccacgttgggtttctatacggctttccaccaagagttttttccacgtgtggaatcccatttcggggtcattgcaaacgagactatggtgtggcgggtgtcacaaacgtctaccttttcagcaggttggtcgcgcttagaatccgatagtaagatcctgcatttgctacgtacttgagactcacagttttttggacacgtctcatgtcctttgttccgacttgtgcaatgaacagcatgcgacgaagtccctagagatggtcgactttagacgacctctacaaccttttttggcaagagagggaagtgtctactttcacagattaggaggctcggttatgatacaatactgttaggtagtttgtagacgtccagagagcgcacaacttccatgagtatgttggtccaatccaagaagggatagatgcttcggtgtggtctgggtagtcccaatctacggtagggtgatttggctctcaagagtaggacgtggtgggatccagtcaccaagtcacgattctgttcgtatgtgatagtatcgcacaactcaaggactttcctctgacctgctgtatcacttaccatgccaatcgtgcgaaccaacagcgggttttgtccataaacgggttgttggcctgggctcctatctcgttgccgctggagactaatgctcgcttacatgtagcattcggactccattgggtccagaagtgtccgtccttgtgaggaggaaccgatactaagcatgcatcttggaccagtggacagcataggatgcacatacacatctccgtaatggtggattcaataagagctgtcatgtgatagcccgtaaccttagctgaggtcctgagacgtgtggtacgtcaatgacttgatgtcgaacctgttctccgagtaagtttggtcgtgttcgaccaaaggcttcagccgaccttacagcctctgtttcgtctcaggcaactcttccgaggtcggattttaccgatacctacgctagtatgtttgccactccagagataacgtgtgggagaccactagggtcagcaaccttggaagccactgagcaagcaggatctttgtcccatcgaggttaggacacctttgtaacctgtgaaacggacacttcatatggctcgtttgctagcctttggtatcttctcgtggtcgcagtcggagtccatagcattgtcgaaggatcgtcctttgaagtctggtctcgagagggacggctagtatcccatcatccggtttcattaatccgcgaccttggacaagattcacctctcgcttccaacctagattgttttctaagcccgcgttggactgtgtgagtacaatgtccggtctgcgtttgctccagctaggggattcacgagctgtagctcagctagagtgcgtaagaagcatgcctaacgtagccgttgacatgcagatcccaaaggtcctgctcgtctcctagactggattaaacgcgatattcgcgtagtcagctgcgtcccctgtcatccttaccctacgatacgacgctcacacacagttacccgcaggaatcgtgcagccgtccgtagagagcttggcgttaattccttagatttgtgtatcccaaaagcaacgcacgagcaccaggacctcgtgagctttgaccggtttatgtgttttgataggaacacgggtccctagaccttgcacgatccaaatgccacacgaaaggtggccatgagtttcgtgacagcgccatatggcacaatcatctgattcgggctaagcatctatcttcgtcgtgtctggttggcagaacgtcggataatggctaatcgtaggttgagacctcccaggttggcactgtagccgattgccaaccattagattcgctatcatgtgctcatgtaggggtctttgcttgcctgagagaaagtgtcaaacgccctatttggtatctcatcgaggtagatatatagacgcgttataattgggggccaagttcgttttcgtaggtgaagatttatcctagcatgtcaggtctaacccaggtttattgaggacaccttctcgtgtgaaggctcctagctgtacacgcctccgtgttacattatgtggcttaccagacttgcctacaattcagtagtgttctttcaaactccagatccgttgagttgacatgcctactggttctgagtacccttagctttggaggatcctagcaatgtaggaatatcactaatccacacaggctttacgcaagcctgacagttcgtgccatgtggaagatcttcgaagatcaggtatcctttacgactggctaaccgaacactcaagagtgtaggtgaattgagaggctccgaagtgagtgatcgggataagtacgaagatcgtggaggcgagcatgttaccatatagctaagtcggcatgtggtacgatgctacactccgattgctcggacacttttctccgctaatacgtcatcctgctagtccgtgatttgtttcgacaacagaccctaagttttttggcatcgtctgtactctcggtctttgagcctcttgcaacgaatcatagacactcaatagatcggaacctttgcttgtcgttcctcaggcatacttttttgacatgctgaggtctggtttcttcttcctccctttcgagacgtacatgcagagaagtttggttactttctgggtttcacatgcttcggcggatgctcataccgaagacactgtcacctgtttcctttgtcggtacctcgactcgttttttgtccacgaggtaggacgtttgtcctaaaggtgtggactccatccaggcgattacagctaggattcgatggttgtttcgccatctccttggtccttttttcgggaaaggagccaggaaaacttttcgaacatcagaacctgcttgacgtcgctatgcacggatcgaactttgatttactcgtgaagacttacagatcctagggcttcgcagtcaagctcatacctttggactagttccatggacttttttgagtctggaacgtacgtgcacgtttagcatgcagtgtatcctagcccaaatgcagaagatcctgccaatttgtgcgattcagactgtgacaggttttttggcttaacagtcagcagtttcatccgtgaactttagctcccagggaggtaggtagtcgttgtagcctaggttgtttacgcacctatccaccacaagtgtgtccagctccgtattctagctttagtgctagcttgagtggagaccttttttctgtgacactcactgggtttgaagaaaatagagacaacgaggctccatgcattagggtgaccctcttgtgtcgtttgtacgcgtggacctggat

SEQ ID NO: 7 - RNA, 1779 nt, synthetic construct
  misc_feature 1..1779: note = source = /note="Description of Artificial Sequence: Synthetic polynucleotide"
  source 1..1779: mol_type = other RNA
gggagaggatccagatgatgtctctatggccaaaagttgaaggtccgactacacgttgcgtaacggtaagcactcatatgagtatgacaggtcatagcagtaaaacgtctatagtccagcaggaaactgctggataggttgggcagtgcaactaggtcgacgaaagtcgagttgcctagtatgaaaagaacagtcgtctcgtgatgtccagcgagtagcagtatcttagagagtcgattctcgcactgggttgaaaatccaagtaacactttgcgaaagcaaagtgggtaactcaaggtttcatctccaccacgaaagtggtatgtgtctgccttaaaattgcggtgcttcataagtgatcgatcgttactgtcagctccagacctttaccactgtgtcagacagaaaatggatacgggtcgttactcaccgctttagtagtgatcagtacttctacttaagaccactagatgctaaaatctggcccaaaagatttggatttgacccatgtccttactgattactcattctcgtcatccgagttcaaaactacgtaggaatgcatcatgcagaactactgagcgatccaataatggttcaggagtgtgtatatctaaaagcgtgtctagcaggtgtggctgggtacacgtctcgaatagacgagcgacgttgggcactatacggtaaaactgtgacactgttgtcggaaacgacaacagacctcaagagattagcaacgtgcacggaaacgtgcgtcgcaaagctacaaaatctagtgcaccttaggaatggtcataatagatgccgtccagctgtcatgacacctagggtcactccaaaactctctaactggtacgagaaatcgtaccactgaccaagggaactagagacagtgtccttctagaaatagaaggacacacatgaacgttccaaaacttggtcagtctccagcgaaagctggagagttagagagggagtgaccctaggttaagtagaagctggacggcatctattatgaccaagtaacgagtgcactagagtagctttgcgacaacgggaaaccgttacgttgctaatctaaaacttgaggtccatgtatcgaaagatacatggtgtcacagaccgctgacacaccaacgtcgctcgtgagctgacgacgtgtacccagcacttatgactagacacgcagatatacacactccatcgactctattggatcgctcagtagttctgcaatcacgagtcctacgtaggaactcggatgacgacatactcaaatcagtaaggacatgggtcaaatctgtagtcgttgggccagaagcatctagtggtctgtcatgacagtactgatcactactaaagcggtgttcctaagcccgtatccactgttatagtgcgtggtaaaggtctgctattcgaagtaacgatcgatccacacctgagcaccgcaaaaggcagacacatgtgcagaaatgcacggagatgaaacctaaaatgagttaccccaacgaagaaattcgttggttacttggacaacccagtgcgagatgaaccattctaagatactgctactcgctggactgatgcatacgactgttccatactaggcaaccgttcgaaagaacgcctagttgcactgaaaacccaacctagccgtagagaaatctacggcctatagacgactgctatgacctgtgaatgagttatgagtgcttaccgttacgcaacgcaaatcttgaccttcaacggccagttcatgttcatctggatcttctcgag

SEQ ID NO: 8 - DNA, 1808 bp, synthetic construct
  misc_feature 1..1808: note = source = /note="Description of Artificial Sequence: Synthetic polynucleotide"
  source 1..1808: mol_type = other DNA
atcggaccttacatcagaacacacgaggtcctaatgtggtgcttcgtgtggagccagattaggcctaaaatcttgaacacgggtctcagtaattgatgatgctgtctgagagcatgccacaactacgggagggtaggccccttgacatcctttctgggttcaccaaataggggatgtgttaggatgtctcggttcttaagctattggtcttcttgattgtcagcctgtcgttcataacagacgagtaactgtgttggcacggcacgtctgttgtcggaatcctgcacaacattctgttgactttcccaagacgaccttccagtgctgctaaatgtatctagcctcaacttgcaaggcgcgtaccgcgaatcgaatgcgttttccatctggaaaattgtgtgaaacgttatgactattgcgcacgaaatgtagagctcagacctcacgtgcgttcgtttgacaataggtggtcgtagttgccatgacgtggcactgtccgaatcggacgtgcagcctgcattagctggcttatttagatttctgaagccggctgttggcagttctcccgattacaatagcccaacagtgcacgtgggcacggcctctggtagggcatgtgcgattgtcgaaagtgggaccaagctctctcgtcggtccgcctggaggtttggacccataagtttgcgagaaatcactgagctctctttctcaacacccagacatccaacgttacgtacgacaagtcttgcagccatagtttcgtggatctcagggagccattcaaatccgactactgggatagagtctggttctattagacgtagaccggtgatgcctataaagcaggggtagttgcggcgactggaaggctcgaagtgccaattgattttcaggctgatctagtttttctagatcagcctgaaaatcaacgttaccttcgagccttccagtcgccgggtgtacccctgctttataggcatcatttccggtctacgtctaataccgtcagactctatcccagtaagacgatttgaatggctccctagactccacgctatggctgcaagacttgtcgtacgtggtcggggatgtctgggtgttgagaaagagaacctcatgatttctcgccttatgggtccaaacgaagacgcggaccgacgagagcgagacgtcccactttctcccatcgcacatgccctaccagaggtttccgtgcccacgtgcactcaccggctattgtaatcgggagaacgtaacgcagccggcttcagaaatctaaataagtttccagctaatgcgcaggacacgtccgattcggatgccgtcacgtcatggcaactcgtctgacctattgtccgaacgcacgtgaggttgaggttctacatttcgtgcgcaatagtcatccgacctcacacaattttccagatggacgcattcgattcgcggtacgcgccttcacacgtgaggctagatacatttagcatgtgtggaaggtcgtcttggggtcaacagaatgttgtaggctgttccgacaacagacgcagtgcgccaacacagttactacgaccttatgaacgacaggcttttgacaatcaactccagcaatagcttaagaacagcttgatcctaacacagacactatttggtgaacccagggatgtcaaggggcctaccctcgaacagttgtggcatgctctcgtcgagcatcatcaattactggagaccgtgttcaagtttattttaggcctaatctggctcgcaagtaagcaccacattaggacctcggcacttctgatgtaaggtccgat

SEQ ID NO: 9 - DNA, 2418 bp, synthetic construct
  misc_feature 1..2418: note = source = /note="Description of Artificial Sequence: Synthetic polynucleotide"
  source 1..2418: mol_type = other DNA
atcttgactggaataacttgtcgatcctcgtgtggcgtttgtcagggtagtacaggtgcggactcaggaacgatgtatcgccatacaccaagctacagtctccaaaggaaaagcgttgcttagtcaccccgacgttcggaccatgatgcgggtgaaaatgcaacgttctccgtagtgatctcggtgagcttggtacgccagtgaagtgcgacggactagtcggatcgtttgtatccacttaccagcaataacaaagtgacgacatcaaaacgtgcgaccgcacagaccagtgttctgtccgctaattgacgtaccactacctaatcgaaacatacctgctcgtatgtgctcgtggatgcttgcggaactgttatgggctttctctttagcggcgatgtctcggctatggagttttggagttctgcaccggccggtgtggtgtggcaaagatcccatttacttctctgcccagtggctgttatcaagccgagcgtcaacccaaacttaactggcagtgctaactgcactgggttctctcacttaaatggacctcggctcgggcagctgggccactcgaatctgcgctcccagataaactcgacccatcacttgcataggtgggctgtcgtgactagcatgttatcctcaagctcggactcgggatcccgtgttccgcacaattcgggcttgagggcacgtgtcccataggtacctacgctggagaaaacagatctgcgaaggacaggctattagctttagatctctgtggcacaacgggttgccattggagctggtataagcatagctcgcatctaggcccaagtctcttctaggttcttcctcgcgtcgagagaagtataaatcgcacccaatccataatacccaacccggcataaagtcctcaaaggattactgcaactgttactgctgattctcggaaatgtgacggtagttacgtacggtaccagacccttgacaatttcgattgggtccgggttcttatcttgatcacacttttcatgatacctatgtgtacacagcctgagccttaactagtttgacgggaaaacacaatagacgcactgttttcagcgaaataggacctgagaggactttgtcaagcatggctgctttgagccgttgggaatcagtgtttatggacggatcttgcacctgacgtctctcacctaaaggttatctagtttttctagataacctttaggtactcgacgtcaggtgcaagatccgttcgttgacactgattcccaacggctcaaagcatttgccatggtccacaaagtcctctcaggttacctttcgctgaaaacagtgtacgtattgtgttttcccgtcctagttaaggctcaggtgggagacacataggtatcatgaaaagtgtgcgagccataagaacccggacccaatcgttgtcaagggtctggtaccgtatgcgactaccgtcacatttcccgtcatcagcagtaagctggtcagtaatcctttgagtttgactttatgccgggttgggtacaacgaattgggtgcgatttatacttcgagtgacgcgaggaagaacctagaagtttagacttgggcctagatgtccactatgcttatagagcagccaatggcaacccgtgtagtgacagagatctgctaatagcctgtcctactgccatctgttttctccaggacgcttacctatgggacacgcactggcaagcccgaattgtgtttcggaacacgggatcccgcaggcgagcttgaggataacatgctctgtgtgacagcccacctatgcaagtgatgggttttcgagtttatcctgtgtcgcagattcgagtggcccagctgccatcaaggaggtccatttaagtgagagaacccatttgtgcagttagctcgcagagttaagtttgggttcgtaggcggcttgataacagctgccctgcagagaagttgggatctttgccacaccacacacctcggtgcagaactccaaaactccatagcgatcacatcgccgctaaagaggcccataacagttccgcaagcacgagcgagcacatacccagctgtatgtttcgattagtgtgccgtacgtcaattagcgtttgacagaacactggtctgcgtagtcgcacgttttgatgtgagaactttgttattcagttgaagtggataccgatccgactacttggtcgcacttcactggcgcctaaagctcaccgagatcaccgtcgagaacgttgcattttcacccgtttcatcatggtccgaacgtcgggacacagaagcaacgcttttcctttggacctggtagcttggtgtatggcgatactttatcgttcctgagtccgccggcgtactaccctgacaaacgccacacgagcgaggacaagttattccagtcaagat

SEQ ID NO: 10 - DNA, 2720 bp, synthetic construct
  misc_feature 1..2720: note = source = /note="Description of Artificial Sequence: Synthetic polynucleotide"
  source 1..2720: mol_type = other DNA
atccacagtagatgcgctttccgctgtcgaagcgactagaatcgataacctcgtcgcctaaattctagcgatctttctcgtgggtatgtaagcaaggaccgagtccatagttgaaatcggacacttctagtcctctgggcataaacagagtcgactggtctcgtacatactgtcaaacgaatgcctgtagaggatacctccacctacgataccataaggatagacattttctgttacacctattcatcaccggtttggctacgtccgtgcagccgttcgtgagtaagaccacatctgacgcgggagaaagcgcatgtgccaagtacagacctagcaagccggacggatgtaattctgcctaggagcaccaggctacgatcgattcgcgtttatagcctcttatctcgagctcgtctcgtctgccacgtacttccgcgcgggtatatagacactttgccaacgtgctgagcgtttccataactaggatatatgtgggttccagtagagagggacaagtcctggttgatggttttatcgccactgggtccagcgaaccaccgatcagcaggatcacatctgcagtagacgcgaaagaggtcgggactcccgtataagtgctaggcaccaaacgcatttaagaatccggacggtctgccggatactcgttcaccgtagccaaaatggcacctgaggctcaaaaccacgaccgcgaacataagaatacttacgctgccatgggacgggtatccctgacacttaggaggcagtcgataaagaagtgcagtgactgcctatcacccttggtctgcccgtctgttagactctatattctgactacccagtggtcacgataccggtgccagcctacgggtctgaagccaggagtcgatgctgaattgtcctttgcgatacagatatgaacgacgtgctaggcgctgtggagtcctactgtactacgggatgtatacgtgtgcctagacaataaaggtaagctatgctaccatcgcaggactattcactaacgtctttcctgagctacataccgatcgatagagttgccttttagtttagatgaacgtcacctaaatatcttccagacaggccgagaactggcacgcatcttcgtatttagtcgacggatgggctcctttgatcctgttggattacgactatggtaccaaagaacgatctctatgagatccactttctccgtgaagaaacaatcccgcacgaggcaactacgtttaccctgttggagagcacttgctgcctgcgcgtcaaaccgtcagagggtggcatctgagacgttgtaccggattgatgggagagtagcgcccagccgagttctagtttttctagaactcggctgggccacgctctcccatcaatccggtacagtttggcagatgccaccctctgacggtttgactttgcgcaggcagcaagtgcaggtcaacagggtaaacgtaggcatctcgtgcgggattgtttcgagacggaggtggatctcatagagaccatggtttggtaccatagtcgtaatccaacgtcgtgaaaggagcccatccgtcgacttacgaagatgctgtgcagttctcggcctgtctctgcgatatttaggtgacgttggtgtaaactaaaaggcaactctatctttgatcggtatgtagctcaggaaccaaactagtgaatagtcctgcgatggcgtgatagcttacctttattgtctagtttgcacacgtatcgcggacgtagtacagtaggaagacgaagcgcctagcagagggttcatatctgtatcgcggacaattcagcatctcgcgttggcttcagacccgttcctgcgcaccggtatcgtgatggaccggtagtcagaatatagtttagtctaacagacgggcatgataagggtgataggcagtcactgtcccagtttatcgactgcctcctaagtgtcagtttggatacccgtctcgttccagcgtaagtattcttatgttcgcgaggatcgttttgagcctcaggtgccattttggtttctacggtgaacgagtatccggacacgggtccggattcttaaatgcgtttggtaggagccacttatacgggagtctttccgacctcttgactccctactgcagatgtgaaggctgtgatcggtggttcgcccactgcagtggcgataccatcaaccaggacttgtccctgcgaactggaacccacatatatcctagttatcagcacgctcagcacgttggcgtgtctatatacccgacatccagtacgtggcagacgctccacgctcgagataacgtcctataaacgcgaatcgatcgtatttgcctggtgctcgctcctagaattacatccgtccggcttgctaccgtgttacttggcacatgcgctttctcccgctttgtcagagtgcgtcttactcacgaacggggaaacggacgtagccaaacccatcatgaataggtgtaacagatgtctatccttatggtatcgttctcggaggtatcctctacagttgctcgtttgacagtatgtacttcaccagtcgacttttctgtttatgcccagaggactactgggatccgatttcaactatggactcatcacttgcttacatacccacgagaatttagatcgctagaatttagctctcgaggttatcgattctagtcgcttcgaggaaggaaagcgcatctactgtggat

SEQ ID NO: 11 - DNA, 3022 bp, synthetic construct
  misc_feature 1..3022: note = source = /note="Description of Artificial Sequence: Synthetic polynucleotide"
  source 1..3022: mol_type = other DNA
atcatgcgtgagccggactcctgtactcattgctaatgtacctatggctaaggagtcgggtacacatatctctttcccgataaacacatctgcgattcggaagcccgtcagctttcgatcgctctgatcacaccggagttggctcttgcttcgtatagtgcagaaagtgcgaattgagtaacctctctcacgaatgagaagacactactgcgtgtcgtcagttgggatttccgctgggtacacagcttcgggtacgctctattcactgcccatacgcggctagtgcctggaaagcaattcaaacgtcacggagtctcctcacgtgggatgagcacccacgtgactaacgaaccactgggacgcggctcgactttgaagtttcatctttaagcccagtaaaccgcagcttcaaacgaaatgttacacgacacatccatcgctcatgaacaaacgcatttgtactccactagaccggatccttacttttccgacggtttccaattgcctgccaagacacaatctaacgtcctcggaacccttagcgacgcacataggtccctcgtgggaccaacccgaaaggagtatggagagtgtcttttggtgggatttaggacctcgcgtccacacatctgcatttgtaccgcgggtgtaagtcacagggcttttcgggatcacttgctaacttttccctattgctattacgaacaggcagacatatgaaaggccaccagtcggcatccggttgctgcacgtcacttgctttccggttgttctcctggtaacaacatgttggctcgtgagtatgcaactgtccaactgtccagtaaagatcttctgatgccaaatcgttcctggacttcattcagttgtatcagtacttacctggtacgtttgttgaaaacaagccttatgcacactttacccagttacgaacggtcatgactctccgttaagagagaagagtgatttgcatcggaaatggacgtttaaggaccttagagtagtaagccataagacagagaacgagaggaatggagctgagccgacatattccactgacaagcaatgccagccgtgtgctgcggcaatagttaactcagctcatgctacactggcctcttgattaaacctctgacaaaagccgcacggactgggcacagtagccgtagcgtgtgatgttcgactgtgcaccagagcttttggtaacgcttttaggtagacgggaaccgggaaaactgtgtgacatgttaaccaatctgccatatacgaggaacgtcccgaagtgactttgcagaacatcatacagctccatgactggcacgtccgcgaagtcggttcgacgcaccttggttggtttccgtcccttataatgtggtggagtaccagtagcaataagtcgggttcctaggctcgcagagttctatccatgtgccgactatgggaccgtctcagcagggagtaggtacgagaccctgaccctcgggcagtgggaatctgcgttctctagtttttctagagaacgcagattcatgttgcccgagggtcagggtctcgtacctaggttctgctgagacggtcccatagtctttggcacatggatagaactagtggagcctaggaacccgacttatgatccatggtactccaccacattataagggactttggaaaccaaccaaggtggtgtgaaccgacttcgcggacaggtcagtcatggagctgtatctgcttctgcgtcacttcgggacgttcctcgtatatcactggttggttaacatgtcacacagttttcgttgcacccgtctacctagcgttaccaaaagccaggcagcacagtcgaatgggacacgctacggctactgccgacagtccgtgcggcttttgtcagtttaggtttaatcaagaggccagttggatctgagctgagttaactattgcccactcacacggctggcattgcttgtctttagtggaatatgtcggctagtgtccattcctctacgtggctgtcttatggcttagaggagtaaggtccttcgtccatttccgatgcaaatcaggcatctctcttaacaccgtctcatgaccgttcgtacggtcttaaagtgtgcataagtttgcttgttttcaacaaaccgctcaggtaagtactgatacaactgaatgacttgcaggaacgatttggcatcagaatttgatctttactctctccttggacagttgcatatggtccagccaacatgttgtttgcgtcagaacaaccgggcaagtgacgtgcagctgcaacatgccgactggtggcctttcatatgccagtgtgttcgtaatagcaatagggagttagcaagtgatcccgaaaagcccttcagagtacacccgcggtacaaatgcaccgatgtggacgcgaggtccttcccaccaaaagacaggacagatactcctttcgggtctcacgcacgagggacctatgaccagggctaagggttccgaggtttacgttagattgtgtcttctctggcaattggaaggagagggaaaagtaaggatcactgggagtggagtactgcgtttgttcatgagcgatggccacgtcgtgtaacatttcgtttgaagctgcctcctactgggcttaaagatgcttcaaagtcgagccgcgtccccagcgttcgttagtccgttctgtgctcatcccacgtctactcactccgtgacgtttgtttaattgctttctctggtctagccgcgtacatccagtgaatagagcgtactgccagctgtgtacccagcggtcccaactgacgacacgcagtacgtccttctcattcgtgagaggtgctactcaattcgcactttgatgactatacgaagtttcaagagccaactccggtgtgagtgactcgatcgaaagctgacgggcttgatgatcgcagatgtgtttatcgggatttaagagatatgtgtaccccaagccttagccataggtacattagcaatgaagcgaggagtccggctcacgcatgat

SEQ ID NO: 12 - DNA, 588 bp, synthetic construct
  misc_feature 1..588: note = source = /note="Description of Artificial Sequence: Synthetic polynucleotide"
  source 1..588: mol_type = other DNA
gaacaggtgagctcataatggcgtacgttcgtacccattttcgtagacactcctcagttttttcaagagagtgggcgtgttgagactacaacaggttttttgtcactgtagtagatcttttcctggcttaaatcaggtcgccggcatctgatactggcatcaggctgtgacggacaaaatcaacttttgacaaagagcacagggttttttgatactgctcgagctctcgggaagcgagttggttttttgaagttcgctttcgggttttgcaagataagaggcaccctagcctcagcgcagcaattattcgttgttgacgaaacgcagtccgttttctccaaggtacataggttttttgcaacgtacccatggtccttgacatgtttaggttttttgcgtgacatgtgtcggttttaagagtggtggacacggacgtcaccttgaagtctgatgcacaactctggacccatgtgtatcattttagatacgagcaatccgttttttgagggtgctcgcccgtggaagagacagtgcggttttttctctcctgtcttggcattttggacttctcgtgcttccacaatgacc

SEQ ID NO: 13 - DNA, 572 bp, synthetic construct
  misc_feature 1..572: note = source = /note="Description of Artificial Sequence: Synthetic polynucleotide"
  source 1..572: mol_type = other DNA
cacctgttcggtcattgtgacacgtcgagaagtcctgccactgtacgagagttttttccgcagtacagcttccttttacgggccttctccctcttttttcggatagaaggtatcttgatacacatgggtcgttcgatgtgcatcagacttcaaggtgacgtcgaattgcaccactcttccgactacacgcacgcttttttcctaacgtgtacaaggttttaccatgccagggttgcttttttcctatcctggttggagcggactgcgtttcgtgctggccgaataattgctgcgctgaggctaggcgttggcttatcttgccccgatggataacttcttttttccaactatccacccgattttgagctctctgcgtatcttttttccctggcagatttgtcgttgattttgtccgtagtccgctgatgccagtatcagatgccggcgagtcacgttaagccagggatctcgattggtgacttttttcctgtcaatcgctcaattttcacgccaccagtcttgttttttctgagctggttctacgtgggtacgaacgtacagacagatgagct

SEQ ID NO: 14 - DNA, 268 bp, synthetic construct
  misc_feature 1..268: note = source = /note="Description of Artificial Sequence: Synthetic polynucleotide"
  source 1..268: mol_type = other DNA
catgacggagattacctcgaactccagctcggataggttttgaggactacaacgtgtaatattttgcctaagggttagcaatcctgtctagctaaaacacgtagttttcgagctctgacgtgacgccatcgatacctcagcgtatcgcctcggactctaccacagagggtattttccttatgcgcccaacggtgtgtggctccgtgcaccaattcctgccagcgttgcttacagcgacttttgcgctcgactcaattctccactgatc

SEQ ID NO: 15 - DNA, 252 bp, synthetic construct
  misc_feature 1..252: note = source = /note="Description of Artificial Sequence: Synthetic polynucleotide"
  source 1..252: mol_type = other DNA
tccgtcatggatcagtggacgtgtcagtcgagcgcgtcgctgtaagcaaccaacaaaggaattggtgcacggagccacacacgtgcctgcgcataaggtaccctctgtggtagcacagcaggcgatacgctgaggtatcgatggccctgattcagagctcgctacgtgttttagctgccattgattgctaacccttaggcaaaatattgaagcatgtagtcctccctatccgagctggacagagtggtaatc

SEQ ID NO: 16 - RNA, 1896 nt, synthetic construct
  misc_feature 1..1896: note = source = /note="Description of Artificial Sequence: Synthetic polynucleotide"
  source 1..1896: mol_type = other RNA
gggagagttgcgaagaatctaacactagtgtacaagggcttctccttatcagagatcatgatgacaacaggtgaatggagggtctaacataagagtaatccaatgcttcgaactaaccttccccttgtggttatcctagcctagcatatacgttctgagaaaacttccctctactcaccatctgcagcaatactctactacaggtctcttttcactctaggggatgcagctaacgacctacgtgttatgaaggtcctagatttgagtagctccactctcgtgcatggggtggacggtaacgcccttctgggtttctcaaaaggggtgttgccagctgaactatggctttggcgtttctttcggaagcgaagctgatgcttgctgaggcagatccactgaaaacccttgcctccagactcgagcttcgcgacagcgtggccggatcatgctccccttagtcagagtcttactgacgtaacaaaacaggcaaatggtctctgtcaagatgcccctttgtgaccgtcgggatgtatgtaacttatgcgtgatttcaccggccatactctggtcgtcattcgctgattgtagggatgccagcggcctcaactactagtggatgtacaaccccggtaatgctctgccataccggtaaggggactataagacccacgtgttactcgcgcgttatatgatggctcatcagccatgttatccaaaagtccagttacctctgactcctcgccgtgctgggactatctcgagtcgcgaactacaatacgagcaattccgcatcccaaaagtccataacttgtattcgatccgcatgctagatgacagtttcttgtcggctgtgaactggttgcgacgtgagctctagcgccctgtgtaacctgaagaagtagttgtgctattgaactcttgtgctagaaccgaatgactcgaagtctagaaatagacttcgacatgtactgttctagcacaagagttcaatagcaatcaggtctcttcaggttacacagggcgctagagaaaactcacgtcgcctaggctacacagccgacaagaggaaggtttctagcatgcggatgattactcagttatggacgggatgcggaattgctcgtattgtagggatcatgtcgagatagtcccagcacggcgaggtgggcttcgtaactggacggataacatggctgaatcccctacatataacgcgcgactgtagtatgggtcttatagtcatggtgagggtatggcagagcataaaataccggggttgtacatccactgacctgataggccgctggcatccctacaatcagagtacatggaccagagtatggccaaaaggtgaaatcagcgttaccttacatacatcccgcgagagtgaaaggggcatcttgtaggacctcatttgcctggttacgtcagtaagagaagcccaaaggggagcatgatccggccacgctcatgatccgctcgagtctggaggcaagggcagtggatctgcctcatgtcatctcagcttcgcttccgaaagaaacgctgtacactaagttcagctggcaacaccccgagaaacccagaaggcgcataaggtccaccccatgcaacggtcacgagctactcaaatcacagagactcataacacgtaggtaaaacgttagctgctgagccatgagtgaaaagagacgtaacacggagtattgctgcagcccttacctagagggaagctcagaacgtatatgaaccagttggataaccacaaggaactgtcaagttcgaagcattgcgaatacattatgttagaccctcaaaacattcacctgtgcaagcaatgatctctgataaggagaagccctcaaagccatgtgttagattcttcgcaactctccc

SEQ ID NO: 17 - RNA, 1684 nt, synthetic construct
  misc_feature 1..1684: note = source = /note="Description of Artificial Sequence: Synthetic polynucleotide"
  source 1..1684: mol_type = other RNA
gggagattactcataagggctggcttggttcactaggagctagttgggtagcccgtcaccagtgcgtacagcccgttcatccgctttgcgattgctcacacaacgcttcgagtttacccgttctgcgattgatcgaaagatcaggacatcgacgggtgaactcgagttgggaagtgagcgatcgcaagcgatcgaaacggaaaactcgctgcttaccgtatgaataggaggtaccttctgccggtagtcgttcgttcagtaagctgagctcgaaagagctgtagtagttgaacggacgactaacttagatcgtagagaccgaggcatacggttccttgaaaaaggacgcaatgacctcggtttctacggtctaagtaaatcaatatcaccactactacctatgccacgaaaacccattgccgaggatccacaatggtgctcacgcgtttatgtagcattttgagcgggatcggttgagagaaatctcatggagttacgctcaagatgctagcacacgccgagcctatagagatggatcctgcttcgaaagaagctcctacggtctctatgggctcggtgtgtgcctagctcgtagctctaactccaatcatggtggaaaatgagtagtccatcgcagagtattcggcctgtgagcgttgttacggatttgctgcagcggatggagtttatgcgaaagcatagactctcgatcgcgcagcagatccgtattcccaaccacaggtcgaataccgatgtccggactgctcaaaaagagcggggttagcatgcgttgccatctcaacatctccgtactgcactctacatgacaagtacgagggtatcttgttcgtgagatcgttcatggtagcacgcagcttcggctgaggagcgatccacaacgctctagaaatagagctggtgacatcgctcttcagccgctcctaggtgctatcatgaacccttatgagaacaaaaagtcgcgtgggccccaatgcctagagctaaatgcgaaaggtgcaagctacgcacagcgtctgataaggcgagtgaaaactcgtcttagttcgtcttgtgcgtggcttgccgcgattccatttagttctaggtcgtctatcccatgcgacaaaaagatatcctccctctgaccatgtagcgtgcagtgcggagaagaggtgtgagacgcgcatgctgcgttgaaaaacgctcgaaaaccgtctcatacctctctccgtgatatcagtaggattcgtcagaggcgcatgaaaatgcggtacttgtgaatcctgctgatattacggagtgttgaggtggcaagttttcgaaacctcgctcccaccgtgataccgatccgagctatgagctagcataaatgcgtgagtaccattgccgtaggacggcgatgggttgcctcagacgcagccctagttatctacctttcgatccttggccacttcattggggacttcgaaagaagtatagacgaaagtggctaaggatgaatcgcgagataattagggctagacgaacggcaaaaaacgtggtatagcagcttacggtgatgttgatttccggcaggaggtacttcctatttcattgcgaagcggcgagaaaagctgtgcgcacgttgtgggggctactcaactagaagctgctgaaccgagccagcgatctcacgtaatctccc

SEQ ID NO: 18 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggtctaaagttttgtcgtgaattgcg

SEQ ID NO: 19 - DNA, 34 bp, synthetic construct
  misc_feature 1..34: note = source = /note="Description of Artificial Sequence: Synthetic oligonucleotide"
  source 1..34: mol_type = other DNA
gtaaagcttttttttttttacaaccaccaccacc

SEQ ID NO: 20 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggtagagcttgacggggaaatcaaaa

SEQ ID NO: 21 - DNA, 34 bp, synthetic construct
  misc_feature 1..34: note = source = /note="Description of Artificial Sequence: Synthetic oligonucleotide"
  source 1..34: mol_type = other DNA
tgtagcattttttttttttacaaccaccaccacc

SEQ ID NO: 22 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggcgagaaaggaagggaacaaactat

SEQ ID NO: 23 - DNA, 34 bp, synthetic construct
  misc_feature 1..34: note = source = /note="Description of Artificial Sequence: Synthetic oligonucleotide"
  source 1..34: mol_type = other DNA
tgagtttctttttttttttacaaccaccaccacc

SEQ ID NO: 24 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggataggaacccatgtacaaacagtt

SEQ ID NO: 25 - DNA, 35 bp, synthetic construct
  misc_feature 1..35: note = source = /note="Description of Artificial Sequence: Synthetic oligonucleotide"
  source 1..35: mol_type = other DNA
caagcccattttttttttttacaaccaccaccacc

SEQ ID NO: 26 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggcaccaccctcattttcctattatt

SEQ ID NO: 27 - DNA, 34 bp, synthetic construct
  misc_feature 1..34: note = source = /note="Description of Artificial Sequence: Synthetic oligonucleotide"
  source 1..34: mol_type = other DNA
ccgccagctttttttttttacaaccaccaccacc

SEQ ID NO: 28 - DNA, 50 bp, synthetic construct
  misc_feature 1..50: note = source = /note="Description of Artificial Sequence: Synthetic oligonucleotide"
  source 1..50: mol_type = other DNA
ggtggtggtggttgtggtggtggtggctacattttgacgctcacctgaaa

SEQ ID NO: 29 - DNA, 34 bp, synthetic construct
  misc_feature 1..34: note = source = /note="Description of Artificial Sequence: Synthetic oligonucleotide"
  source 1..34: mol_type = other DNA
ccctcagttttttttttttacaaccaccaccacc

SEQ ID NO: 30 - DNA, 26 bp, synthetic construct
  misc_feature 1..26: note = source = /note="Description of Artificial Sequence: Synthetic oligonucleotide"
  source 1..26: mol_type = other DNA
ggtggtggtggttgtggtggtggtgg

(19) United States
(12) Patent Application Publication
(10) Pub. No.: US 2025/0340891 A1
(43) Pub. Date: Nov. 6, 2025

(54) HIGHLY KNOTTED MOLECULAR TOPOLOGIES FROM SINGLE-STRANDED NUCLEIC ACIDS

(71) Applicant: ARIZONA BOARD OF REGENTS ON BEHALF OF ARIZONA STATE UNIVERSITY, Scottsdale, AZ (US)

(72) Inventors: Hao YAN, Chandler, AZ (US); Fei ZHANG, Chandler, AZ (US); Xiaodong QI, Tempe, AZ (US)

(21) Appl. No.: 19/217,148

(22) Filed: May 23, 2025

Related U.S. Application Data
(63) Continuation of application No. 17/050,918, filed on Oct. 27, 2020, now Pat. No. 12,344,845.
(60) Provisional application No. 62/663,678, filed on Apr. 27, 2018.

(51) Int. Cl. C12N 15/117; C07H 21/00

(52) U.S. Cl. CPC C12N 15/117; C07H 21/00; C12N 2310/17; C12N 2310/18

(57) ABSTRACT

In some embodiments, complex molecular knots with high crossing numbers are achieved by folding, following a prescribed folding order, single-stranded DNA or RNA of customized sequences into target shapes. Such complex molecular knots with high crossing numbers are useful for biomedical applications including use as immunostimulatory agents and/or protein hosts and carriers.

### RELATED APPLICATIONS

This application is a continuation of U.S. application Ser. No. 17/050,918, filed Oct. 27, 2020, which is a 371 National Phase Entry of PCT/US2019/029201, filed Apr. 25, 2019, which claims benefit of U.S. Provisional Application No. 62/663,678, filed on Apr. 27, 2018, the entire disclosure of which is incorporated herein by reference.

### GOVERNMENT FUNDING

This invention was made with government support under N00014-15-1-2689 awarded by the Office of Naval Research and 1360635, 1563799, 1334109 awarded by the National Science Foundation. The government has certain rights in the invention.

### SEQUENCE LISTING

The instant application contains a Sequence Listing, which has been submitted electronically in XML format and is hereby incorporated by reference in its entirety. Said XML copy, created on May 19, 2025, is named G8118-01303_SL.xml and is 88,047 bytes in size.

### BACKGROUND

Topological knots are complex structures which are formed from continuous loops with a number of crossover events. Molecular knots are present in biopolymers such as DNA and proteins. (M. L. Mansfield, Nat Struct Biol 1, 213-214 (1994); F. Takusagawa, et al., J Am Chem Soc 118, 8945-8946 (1996); W. R. Taylor, Nature 406, 916-919 (2000); J. R. Wagner, et al., Nature 438, 325-331 (2005); J. J. Champoux, Annu Rev Biochem 70, 369-413 (2001); L. F. Liu, et al., Nucleic Acids Res 9, 3979-3989 (1981)). During protein folding, the protein molecules occasionally exhibit small amounts of strand-crossing structures. DNA knots can be formed during DNA replication and are eventually resolved by DNA topoisomerases. While DNA knots are present in some bacterial phage genomes during DNA packing, the DNA knots are not predictable or well organized (L. F. Liu, et al., P Natl Acad Sci-Biol 78, 5498-5502 (1981)).

Constructing synthetic molecular knots presents an extraordinarily high level of control over the objects and their assembly behaviors at nanometer scales. (R. S. Forgan, et al., Chem Rev 111, 5434-5464 (2011)). It is challenging to design and construct highly knotted nanostructures with well-defined geometries. DNA synthesis technologies allows for the programmability of nucleic acid sequences imparting both information and function. The programmability of nucleic acids makes them suitable candidates for creating arbitrary knots at the molecular level. A diversity of design techniques and approaches for DNA self-assembly have been exploited, resulting in a wide variety of nanostructures that exhibit geometric complexity that is comparable to or even more complex than that found in nature.

### SUMMARY

In some aspects, this disclosure provides for a knotted, self-assembled single-stranded nucleic acid (ssNA) nanostructure comprising a crossing number from 1 to 1200 and comprising at least one paranemic cohesion crossover. In some aspects, the nanostructure comprises a crossing number from 2 to 100 crossings. In some aspects, the nanostructure comprises a crossing number from 9 to 67 crossings. In some aspects, the nucleic acid is selected from DNA, RNA, or combinations thereof.

In some aspects, the ssNA nanostructure comprises an NA sequence of about 1000 to about 10,000 nucleotides in length. In some aspects, the NA sequence is about 1800 to about 7500 nucleotides in length. In some aspects, the NA sequence is about 6500 to about 7500 nucleotides in length.

In some aspects, the knotted ssNA nanostructure self-assembles into a 3-dimensional shape. In some aspects, the nanostructure self-assembles by means of paranemic cohesion into knots comprising four ssNA regions.

In some aspects, the knotted ssNA nanostructure further comprises a plurality of paranemic adhesion crossovers which occur in one or a plurality of periodic frequencies. In some aspects, the one or a plurality of periodic frequencies is selected from every 6, 7, 8, 9, 10, 11, 12, 13, 14, 15, 16, 17, 18, 19, or 20 bp.

In some aspects, the folding efficiency of the nanostructure is greater than 50%. In some aspects, the folding efficiency of the nanostructure is greater than 85%. In some aspects, the folding efficiency of the nanostructure is greater than 90%. In some aspects, the folding efficiency of the nanostructure is greater than 91%, 92%, 93%, 94%, 95%, or higher.

In some aspects, the knotted ssNA nanostructure comprises at least about 75% sequence identity to SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16 or SEQ ID NO:17.

In some aspects, the knotted ssNA nanostructure comprises at least about 85% sequence identity to SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO: 10, SEQ ID NO: 11, SEQ ID NO:12, SEQ ID NO:13, SEQ ID NO:14, SEQ ID NO: 15, SEQ ID NO: 16 or SEQ ID NO:17.

In some aspects, the knotted ssNA nanostructure comprises at least about 95% sequence identity to SEQ ID NO: 1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO:10, SEQ ID NO: 11, SEQ ID NO:12, SEQ ID NO: 13, SEQ ID NO: 14, SEQ ID NO: 15, SEQ ID NO: 16 or SEQ ID NO:17.

In some aspects, the knotted ssNA nanostructure comprises at least about 99% sequence identity to SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO: 10, SEQ ID NO: 11, SEQ ID NO:12, SEQ ID NO:13, SEQ ID NO: 14, SEQ ID NO: 15, SEQ ID NO: 16 or SEQ ID NO:17.

In some aspects, the knotted ssNA nanostructure comprises SEQ ID NO:1,SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16 or SEQ ID NO:17.

In some aspects, the knotted ssNA nanostructure comprises DNA having 9 crossings forming a square knotted DNA.

In some aspects, the knotted ssNA nanostructure comprises a nucleic acid having the sequence of SEQ ID NO: 1. In some aspects, the knotted ssNA nanostructure consists of a nucleic acid having the sequence of SEQ ID NO:1.

In some aspects, the knotted ssNA nanostructure comprises DNA having 23 crossings forming a 3-square knotted DNA before hierarchical design.

In some aspects, the knotted ssNA nanostructure comprises a nucleic acid having the sequence of SEQ ID NO:2 or SEQ ID NO: 3. In some aspects, the knotted ssNA nanostructure consists of a nucleic acid having the sequence of SEQ ID NO:2 or SEQ ID NO: 3.

In some aspects, the knotted ssNA nanostructure comprises DNA having 57 crossings forming a 9-square knotted DNA before hierarchical design.

In some aspects, the knotted ssNA nanostructure comprises a nucleic acid having the sequence of SEQ ID NO:4. In some aspects, the knotted ssNA nanostructure consists of a nucleic acid having the sequence of SEQ ID NO:4.

In some aspects, the knotted ssNA nanostructure comprises DNA having 57 crossings forming a 9-square knotted DNA after hierarchical design.

In some aspects, the knotted ssNA nanostructure comprises a nucleic acid having the sequence of SEQ ID NO:5. In some aspects, the knotted ssNA nanostructure consists of a nucleic acid having the sequence of SEQ ID NO:5.

In some aspects, the knotted ssNA nanostructure comprises DNA having 67 crossings forming a hexagonal knotted DNA.

In some aspects, the knotted ssNA nanostructure comprises a nucleic acid having the sequence of SEQ ID NO:6. In some aspects, the knotted ssNA nanostructure consists of a nucleic acid having the sequence of SEQ ID NO:6.

In some aspects, the knotted ssNA nanostructure comprises RNA having 9 crossings forming a square knotted RNA. In some aspects, the knotted ssNA nanostructure comprises a sequence encoded by or complementary to a nucleic acid having the sequence of SEQ ID NO:7. In some aspects, the knotted ssNA nanostructure consists of a sequence encoded by or complementary to a nucleic acid having the sequence of SEQ ID NO:7.

In some aspects, the knotted ssNA nanostructure comprises DNA having 15crossings forming a tetrahedron. In some aspects, the knotted ssNA nanostructure comprises a nucleic acid having the sequence of SEQ ID NO:8 or SEQ ID NO: 17. In some aspects, the knotted ssNA nanostructure consists of a nucleic acid having the sequence of SEQ ID NO:8 or SEQ ID NO:17.

In some aspects, the knotted ssNA nanostructure comprises DNA having 20 crossings forming a pyramid. In some aspects, the knotted ssNA nanostructure comprises a nucleic acid having the sequence of SEQ ID NO:9. In some aspects, the knotted ssNA nanostructure consists of a nucleic acid having the sequence of SEQ ID NO:9.

In some aspects, the knotted ssNA nanostructure comprises DNA having 22 crossings forming a triangular prism. In some aspects, the knotted ssNA nanostructure comprises a nucleic acid having the sequence of SEQ ID NO:10. In some aspects, the knotted ssNA nanostructure consists of a nucleic acid having the sequence of SEQ ID NO:10.

In some aspects, the knotted ssNA nanostructure comprises DNA having 25 crossings forming a pentagonal pyramid. In some aspects, the knotted ssNA nanostructure comprises a nucleic acid having the sequence of SEQ ID NO:11. In some aspects, the knotted ssNA nanostructure consists of a nucleic acid having the sequence of SEQ ID NO:11.

In some aspects, the knotted ssNA nanostructure further comprises a topological control strand comprising a sequence selected from a nucleic acid having the sequence of SEQ ID NO: 12, SEQ ID NO:13, SEQ ID NO: 14 or SEQ ID NO:15. In some aspects, the knotted ssNA nanostructure further comprises a topological control strand consisting of a sequence selected from a nucleic acid having the sequence of SEQ ID NO:12, SEQ ID NO:13, SEQ ID NO:14 or SEQ ID NO:15.

In some aspects, the knotted ssNA nanostructure further comprises a 5′-end and a 3′-end, wherein the 5′-end and/or the 3′-end further comprises 1-5 nucleotide terminal extensions. In some aspects, both the 5′-end and the 3′-end of the knotted ssNA nanostructure comprise terminal extensions and the terminal extensions hybridize to form a single strand of nucleic acid.

In some aspects, the knotted ssNA nanostructure further comprises at least one diagnostic agent operably linked to said nanostructure.

In some aspects, the knotted ssNA nanostructure further comprises at least one therapeutic agent operably linked to said nanostructure. In some aspects, at least one therapeutic agent is an anti-tumor agent. In some aspects, the at least one therapeutic agent is a chemotherapeutic drug.

In some aspects, the nanostructure is replicable. In some aspects, the replication occurs in a transformed cell.

In some aspects, this disclosure relates to a pharmaceutical composition comprising a knotted, self-assembled single-stranded nucleic acid (ssNA) nanostructure comprising a plurality of crossings and comprising at least one paranemic cohesion crossover, and a pharmaceutically acceptable carrier.

In some aspects, this disclosure relates to a method of inducing an immune response in a subject, comprising administering to the subject an effective amount of a knotted ssNA nanostructure described herein. In some aspects, the knotted ssNA nanostructure comprises a knotted, self-assembled single-stranded nucleic acid (ssNA) nanostructure comprising a plurality of crossings and comprising at least one paranemic cohesion crossover.

In some aspects, this disclosure relates to a method of treating a disease or disorder in a subject, comprising administering to the subject a therapeutically effective amount of a knotted ssNA nanostructure as described herein or a pharmaceutical composition as described herein. In some aspects, the knotted ssNA nanostructure comprises a knotted, self-assembled single-stranded nucleic acid (ssNA) nanostructure comprising a plurality of crossings and comprising at least one paranemic cohesion crossover. In some aspects, the disease or disorder is cancer. In some aspects, the cancer is breast cancer.

In some aspects, the method further comprises administering at least one therapeutic agent to the subject. In some aspects, the at least one therapeutic agent is a tumor targeting agent. In some aspects, the tumor targeting agent is selected from a monoclonal tumor-specific antibody or an aptamer.

In some aspects, this disclosure relates to the use of a knotted ssNA nanostructure as described herein or a composition as described herein for the manufacture of a medicament for inducing an immune response in a subject.

In some aspects, this disclosure relates to a nanostructure for inducing an immune response in a subject.

In some aspects, this disclosure relates to a use of a knotted ssNA nanostructure as described herein or a composition as described herein for the manufacture of a medicament for treating a disease or disorder in a subject.

In some aspects, this disclosure relates to a knotted ssNA nanostructure as described herein or a composition as described herein for the prophylactic or therapeutic treatment of a disease or disorder in a subject.

In some aspects, this disclosure relates to a kit comprising a knotted ssNA nanostructure as described herein or a composition as described herein and instructions for administering the nanostructure/composition to a subject to induce an immune response or to treat a disease or disorder. In some aspects, the kit further comprises at least one therapeutic agent.

In some aspects, this disclosure provides a single-stranded nucleic acid (ssNA) nanostructure comprising from 9 to 67 crossings and comprising at least one paranemic cohesion crossover, wherein the nanostructure is self-assembled. In some aspects, the nanostructure is replicable. In some aspects, the nanostructure is a topological knot.

In some aspects, the nucleic acid is DNA

In some aspects, the nucleic acid is RNA.

In some aspects, the nanostructure comprises an NA sequence of about 1000 to about 10,000 nucleotides in length.

In some aspects, the NA sequence is about 1800 to about 7500 nucleotides in length.

In some aspects, the nanostructure comprises at least about 75% sequence identity to SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, or SEQ ID NO: 17.

In some aspects, the nanostructure comprises at least about 85% sequence identity to SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO: 11, SEQ ID NO:12, SEQ ID NO:13, SEQ ID NO:14, SEQ ID NO:15, SEQ ID NO:16, or SEQ ID NO: 17.

In some aspects, the nanostructure comprises at least about 95% sequence identity to SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO: 16, or SEQ ID NO: 17.

In some aspects, the nanostructure comprises at least about 99% sequence identity to SEQ ID NO:1, SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, or SEQ ID NO: 17.

In some aspects, the nanostructure comprises SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO: 13, SEQ ID NO: 14, SEQ ID NO:15, SEQ ID NO:16, or SEQ ID NO:17.

In some aspects, the nanostructure comprises DNA having 9 crossings forming a square knotted DNA.

In some aspects, the nanostructure comprises SEQ ID NO:1.

In some aspects, the nanostructure consists of SEQ ID NO:1.

In some aspects, the nanostructure comprises DNA having 23 crossings forming a 3-square knotted before hierarchical design.

In some aspects, the nanostructure comprises SEQ ID NO:2.

In some aspects, the nanostructure consists of SEQ ID NO:2.

In some aspects, the nanostructure comprises SEQ ID NO:3.

In some aspects, the nanostructure consists of SEQ ID NO:3.

In some aspects, the nanostructure comprises DNA having 57 crossings forming a 9-square knotted DNA before hierarchical design.

In some aspects, the nanostructure comprises SEQ ID NO:4.

In some aspects, the nanostructure consists of SEQ ID NO:4.

In some aspects, the nanostructure comprises DNA having 57 crossings forming a 9-square knotted DNA after hierarchical design.

In some aspects, the nanostructure comprises SEQ ID NO:5.

In some aspects, the nanostructure consists of SEQ ID NO:5.

In some aspects, the nanostructure comprises DNA having 67 crossings forming a hexagonal knotted DNA.

In some aspects, the nanostructure comprises SEQ ID NO:6.

In some aspects, the nanostructure consists of SEQ ID NO:6.

In some aspects, the nanostructure comprises RNA having 9 crossings forming a square knotted RNA.

In some aspects, the nanostructure comprises a sequence encoded by or complementary to SEQ ID NO:7.

In some aspects, the nanostructure consists of a sequence encoded by or complementary to SEQ ID NO:7.

In some aspects, the nanostructure comprises DNA having 15 crossings forming a tetrahedron.

In some aspects, the nanostructure comprises SEQ ID NO:8.

In some aspects, the nanostructure consists of SEQ ID NO:8.

In some aspects, the nanostructure comprises DNA having 20 crossings forming a pyramid.

In some aspects, the nanostructure comprises (SEQ ID NO:9).

In some aspects, the nanostructure consists of (SEQ ID NO:9).

In some aspects, the nanostructure comprises DNA having 22 crossings forming a triangular prism.

In some aspects, the nanostructure comprises (SEQ ID NO:10).

In some aspects, the nanostructure consists of (SEQ ID NO:10).

In some aspects, the nanostructure comprises DNA having 25 crossings forming a pentagonal pyramid

In some aspects, the nanostructure comprises SEQ ID NO:11.

In some aspects, the nanostructure consists of SEQ ID NO:11.

In some aspects, the nanostructure comprises topological control strand SEQ ID NO: 12, SEQ ID NO:13, SEQ ID NO:14 or SEQ ID NO:15.

In some aspects, the nanostructure consists of topological control strand SEQ ID NO: 12, SEQ ID NO:13, SEQ ID NO:14 or SEQ ID NO:15.

In some aspects, the nanostructure has a 5′-end and a 3′-end, wherein the 5′-end and/or the 3′-end further comprise 1-5 nucleotide terminal extension.

In some aspects, both the 5′-end and the 3′-end comprise terminal extensions and the terminal extension hybridize to form a single strand of nucleic acid.

In some aspects, at least one diagnostic agent operably linked to the nanostructure.

In some aspects, at least one therapeutic agent is operably linked to the nanostructure.

In some aspects, the at least one therapeutic agent is a tumor antigen peptide.

In some aspects, this disclosure provides a pharmaceutical composition comprising the nanostructure described herein and a pharmaceutically acceptable carrier.

In some aspects, the pharmaceutical composition further comprising at least one therapeutic agent.

In some aspects, the at least one therapeutic agent is a chemotherapeutic drug (e.g., doxorubicin).

In some aspects, this disclosure provides a method of inducing an immune response a subject (e.g., a mammal, which can include or exclude a human), comprising administering to the subject an effective amount of a nanostructure or a composition as described herein.

In some aspects, this disclosure provides a method of treating a disease or disorder in a subject, comprising administering to the subject a therapeutically effective amount of a nanostructure or a composition as described herein.

In some aspects, the disease or disorder is cancer.

In some aspects, the cancer is breast cancer.

In some aspects, the method further comprises administering at least one therapeutic agent to the subject.

In some aspects, the at least one therapeutic agent is a tumor targeting agent (e.g., a monoclonal tumor-specific antibody, antibody fragment or an aptamer).

In some aspects, this disclosure provides a use of a nanostructure or composition as described herein for the manufacture of a medicament for inducing an immune response in a subject (e.g., a mammal, which can include or exclude a human).

In some aspects, this disclosure provides a nanostructure or composition as described herein for inducing an immune response.

In some aspects, this disclosure provides the use of a nanostructure or composition as described herein for the manufacture of a medicament for treating a disease or disorder in a subject.

In some aspects, this disclosure provides a nanostructure or composition as described herein for the prophylactic or therapeutic treatment a disease or disorder.

In some aspects, this disclosure provides a kit comprising a nanostructure or composition as described herein and instructions for administering the nanostructure/composition to a subject to induce an immune response or to treat a disease or disorder.

In some embodiments, the kit further comprises at least one therapeutic agent.

### BRIEF DESCRIPTION OF DRAWINGS

FIGS. 1A-1D. Design of single-stranded DNA/RNA knots. A knot with a crossing number of 9 is constructed via one of two methods: FIG. 1A depicts the first method which is to assemble with preformed individual X-nodes that are linked together with specific sticky ends associations and ligation. FIG. 1B depicts the second method where a single chain is threaded and knotted into the target topology. FIG. 1C depicts paranemic crossover motifs which were introduced as the building blocks for the knotted nucleic acid nanostructures. FIG. 1D A schematic diagram that shows the design and folding pathway of a single-stranded DNA to form knot 9₁ as an example. A single chain DNA was assigned with partially paired regions to first form a large loop-stem hairpin structure. Then, the unpaired loop regions were designed to interact with each other through paranemic cohesions to form the target knot. The formation of the knot involved the threading of the two ends of the loop-stem structure by following a pathway similar to that shown in FIG. 1B.

FIGS. 2A-2E. Design and AFM characterization of two-dimensional single-stranded DNA and/or RNA knots. Designer models (top row) for the 2D nanostructures and their corresponding AFM images (middle row shows the zoomed-in images and bottom row shows the zoomed-out images) with increasing crossing numbers: FIG. 2A shows a 9 crossing DNA square, FIG. 2B shows a 9 crossing RNA square, FIG. 2C shows a 23 crossing DNA rectangle with 3 square cavities, FIG. 2D shows a 57 crossing DNA 3×3 square lattice, and FIG. 2E shows a 67 crossing DNA hexagonal lattice.

FIGS. 3A-3D. Optimization of the folding pathway for ssDNA knots. (FIG. 3A) A designed model shows the best folding pathway for the three-square structure with 23 crossings, by following the optimization rules. The red to gray color scale represent the order of the paranemic cohesion interaction strengths on the edges based on the number and length of the paranemic cohesions involved. FIG. 3B shows the paranemic interaction regions are designed with lengths of 4 bp or 6 bp with distinct expected binding strengths (6 bp>4 bp). The folding order of the knot structure is guided by controlling the sequences and lengths of the paranemic interactions in each individual edge. FIG. 3C shows that the folding efficiency of the various structures was compared by using different folding pathways before and after optimization. FIG. 3D shows that the AFM images revealed a dramatic increase in the the folding yield of well-form structures from 0.9% (N=221) to 57.9% (N=214).

FIGS. 4A-4B. Topological control experiments. FIG. 4A shows a DNA link structure with linking number of 8 was designed and constructed from four linear ssDNA (top row and bottom left image). As used herein, the term “linking number” refers to the number of links within a knotted NA nanostructure. High resolution AFM images confirmed the successful formation of the target structures (bottom right images). FIG. 4B shows that the four linear ssDNA strands were first ligated into two closed circles, and the assembled final structures from the two circles showed obvious defects under AFM imaging.

FIGS. 5A-5D. Design topologies and cryo-EM reconstruction of three- dimensional single-stranded DNA knots. A schlegel diagram (left panel) transforms a three-dimensional object into a two-dimensional diagram. By following the same design rules of two-dimensional knots, several three-dimensional DNA knot frameworks were designed (middle panels of two different view angles for each structure). The cryo-EM reconstruction (right panel) revealed the correct formations of a tetrahedron with crossing number 15 (FIG. 5A), a square pyramid with crossing number 20 (FIG. 5B), a triangular prism with crossing number 22 (FIG. 5C), and a pentagonal pyramid with crossing number 25 (FIG. 5D).

FIGS. 6A-6D. The design of a DNA knot 9₁. FIG. 6A. The schematics show how to assign 9 crosses on a square geometry. As each edge has the same length, well-distributed crosses are preferred to maintain the stability of the DNA knotted nanostructure. FIG. 6B. Paranemic cohesion interaction, each contains two parallel crossovers with 4 bp or 6 bp. These base pairs represent the cross in the knot design schematics. The distance between the adjacent paranemic cohesion crosses are designed as integer multiples of one DNA helical turn (10, 11, 21 or 32 bp). A linking structure comprised of two ssDNA loops and one crossover was designed to connect DNA strands in each outer corner. FIG. 6C. An arrangement of each of the edges of the square with the corresponding DNA knotted nanostructures. FIG. 6D. Adding small linking structures at the vertexes finishes the design of the structure. The DNA strands in the inner corners are connected directly using poly T loops (T4 is used for a 90 degree turn in the square). The outer corners are linked with the linking structure containing one paranemic cohesion interaction.

FIGS. 7A-7B. A schematic of the folding pathway design. FIG. 7A. An edge with an odd numbers of crosses enables the two loops that form the edge to connect into one large loop, i.e. to form a knot structure, while an edge with even numbers of crosses produces a link between two separate loops. The schematics show an edge with 3 crosses that formed a knot of 31, and an edge of 2 crosses that resulted in a link structure called a Hopf link. FIG. 7B. To form a knot structure instead of a link, an odd number of crossings among the individual loops was required and different orders of connections to link the loops (a, b, c, c) was selected to produce the target knot. The loops are designated according to their geometric relationships, and the two c loops are the same due to their symmetry. The largest loop a is connected with either loops b or c, while c is only connected with loops a or b, not with the other loop c. All of the possible orders of connections among the loops is listed, which represents different folding pathways that could form the target knot structures. Pathways 1 and 3 are branched and pathways 2,4, and 5-8 are linear.

FIGS. 8A-8B. A comparison between a linear folding pathway (FIG. 8A) and a branched folding pathway (FIG. 8B). According to the connection relationship between the loops, a to c, the edges were designed with either 3 crosses or 2 crosses, (with the total number of the crosses being an odd number), to form the knot. The edges with 3 crosses are marked with an X. These edges form earlier than the edges with 2 crosses, due to the annealing step and the difference in the strength of the paranemic cohesions involved. The pathways represent the order of the formation of the three cross edges, i.e. the formation of the corresponding loops. The direction of the linear pathway c-b-c-a (FIG. 8A) is reversed as a-c-b-c without changing the relationships of loop connection. However, these two linear pathways are not equivalent. The loop sequence c-b-c-a was identified as a preferred direction because the two ends will not need to thread into any preformed loops during the early steps, which is when the unfolded strand is still long. For the branched pathway (FIG. 8B), the two ends need to be separated after forming the first 3-cross edges. Each end then travels individually and threads through a preformed loop (the central one) to create the 2-cross edges to form the loops on the sides. The branched path is expected to be less favorable than the linear one because the formation of the 2-cross edges is expected to occur later than the 3-cross edges due to thermodynamic reasons. Among the linear paths, the path should avoid threading through pre-formed structures when the unfolded strand is long. Due to these reasons, path 5 (depicted in FIG. 8A) is more efficient at forming self-assembled knotted nanostructures better than paths 3 or 4 shown in FIGS. 7A-7B.

FIGS. 9A-9D. The sequence design for the hierarchical folding based on the selected best pathway. FIG. 9A. A schematic of the cross section of the c-b-c-a folding path (as shown in FIGS. 7A-7B, 8A-8B). The ends of the partially folded dsDNA are located at the upper left corner. The gradual color change from red to grey represents the order of the looping. FIG. 9B. The folding order of all of the edges are labeled as steps 1 to 7. The edges that are marked as 1-3 are the 3-cross edges while 4-7 are the 2-cross edges. FIG. 9C. A knotted DNA nanostructure that represents the target topological geometry. The white numbers are the length (bp) of each paranemic cohesion interaction. FIG. 9D. A summary table of the design parameters for the structure, including the number of bases in each paranemic cohesion, and the GC content of the paranemic cohesions in each edge.

FIGS. 10A-10B. A comparison between the yield of the three-square knot structure 9₁ and the different folding pathways, via the use of AFM imaging. With an unfavorable folding pathway (FIG. 10A), the folding yield is only 0.9% (2/221). With the best folding pathway (FIG. 10B), the folding yield is increased to 57.9% (124/214). As used herein, the term “folding yield” refers to the number of nanostructures which have adopted the designed nanostructure compared to the number of nanostructures which have not adopted the designed nanostructure.

FIGS. 11A-11B. The replication and production of an ssDNA by using a recombinant M13 phage. FIG. 11A. The ssDNA replication process. First, the two halves of the ssDNA gene (red and blue), were obtained by restriction enzyme digestion from a commercially synthesized plasmid. Then, the custom synthesized ssDNA gene was ligated into a phagemid vector pGEM-7zf(−) (black) by T4 DNA ligase (New England Biolabs) and co-transformed into E. coli DH5α competent cells (New England Biolabs) with the helper plasmid pSB4423 (orange). During the phage replication, the ssDNA sequence (red and blue) was packed into the phage capsid as its genome. Recombinant phages were then harvested from the E. coli medium and the recombinant phage genomic DNA was isolated and purified. The EcoRV restriction sites were initially designed at the ends of the ssDNA and the phage DNA digestion by EcoRV restriction enzyme produced the ssDNA molecule (partially paired and folded into a hairpin, with the 5′ and 3′ ends meeting each other and the unpaired bubbles as paranemic cohesion sites). FIG. 11B. An example of the 1800 nt ssDNA purification by gel electrophoresis. Lane I represents the 1 kb dsDNA ladder. Lane 2 contains the purified phage DNA without EcoRV cleavage. After EcoRV digestion, the 1800 nt ssDNA molecule (lower band) is separated from the vector DNA (upper band) in lane 3. The 1800 nt ssDNA molecule runs slightly faster than the 1 kb dsDNA (2000 nt).

FIGS. 12A-12B. The design and characterization of the square knotted DNA nanostructure 9₁. FIG. 12A shows the design schematic of the square knotted DNA nanostructure 9₁. FIG. 12B shows representative AFM images of the square knotted DNA nanostructure 9₁.

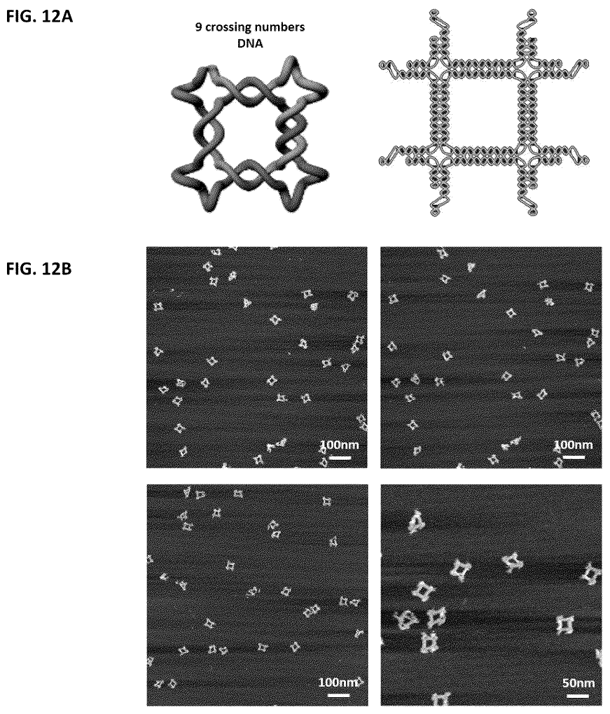

FIGS. 13A-13B. The design and characterization of the 9-square knotted DNA nanostructure. FIG. 13A shows the design schematic of the 9-square knotted DNA nanostructure. FIG. 13B shows representative AFM images of the 9-square knotted DNA nanostructure.

FIGS. 14A-14B. The design and characterization of the hexagonally knotted DNA nanostructure. FIG. 14A shows the design schematic of the hexagonally knotted DNA nanostructure. FIG. 14B shows representative AFM images of knotted DNA nanostructures of the hexagonally knotted DNA nanostructure.

FIGS. 15A-15C. The design and characterization of the 9-square knotted DNA nanostructure with hierarchical folding. FIG. 15A depicts the folding pathway design. The numbers on the edges mark the anticipated order of the formation of the crosses on the edges, based on the designed sequences. FIG. 15B depicts the sequence design. The length of the paranemic cohesion, the number of the base pairs involved, and the GC content of the base pairs are all tuned to make the expected strength of the paranemic cohesion interactions follow the anticipated folding order. FIG. 15C shows representative AFM images of the folded knot structure (with 57 crossings). A majority (if not all) of the structures formed show some degree of errors. It seems that if the crossings in some of the earlier steps did not form properly, the crossings in the later steps could still form, but that the errors would be permanently trapped and there would be no chance of correcting the errors. Since each crossing may have a certain rate of error, with 57 crossings, the final product is expected to have a low yield. Nevertheless, the stepwise yield is quite high (>90%) with the overall structures having a high resemblance to the expected design. Most of the structures have more than half of their edges properly formed.

FIGS. 16A-16B. Topological control with linear and circular DNA. FIG. 16A shows two linearly annealed and partially paired dsDNAs, (with internal loops for paranemic cohesions between the two DNA), can self-assemble into the designed structure with a high yield, as shown in the AFM images of the self-assembled knotted NA nanostructures. A small number of the final structures show mild defects. FIG. 16B shows that after circularization, the two circular dsDNA molecules cannot form the nanostructure. The AFM images show that all of the final structures contain moderate to extensive defects.

FIGS. 17A-17C. The design and characterization of the square knotted RNA nanostructure 9₁. FIG. 17A shows the design schematics of the square knotted RNA nanostructure 9₁. In some embodiments, an a-form double helix is the structural model. FIG. 17B shows an RNA paranemic cohesion design that was based on A-form double helices. 8 bp was chosen as the length of the paranemic cohension for RNA; 33 bp was chosen as the length of the repeating unit (3 full turns). FIG. 17C shows representative AFM images of the self-assembled knotted NA nanostructures, illustrating a high folding yield (˜60%) of the expected structure.

FIGS. 18A-18D. The design and characterization of the tetrahedron knotted DNA nanostructure. FIG. 18A. The design schematics of the folding pathway. In the middle 2D diagram, the red dots mark the edges of the tetrahedron that have the 3 crossing numbers. The number on the edges mark the anticipated order of formation of the edges. FIG. 18B shows the vertexes all show the illustrated chirality. FIG. 18C shows the sequence design parameters that facilitate the folding order. FIG. 18D shows a representative AFM image of the self-assembled knotted NA nanostructures. Most of the structures in the image display the expected structure. However, some of the structures shown are missing one or two edges.

FIGS. 19A-19C. The design and characterization of a pyramid knotted DNA nanostructure (crossing number=20). FIG. 19A shows the design schematics of the folding pathway. FIG. 19B shows the sequence design parameters that facilitate the folding order. FIG. 19C shows representative AFM images. However, although many of the structures that are shown are distorted or broken, due to interactions with the substrate surface, a majority of the edges shown are well formed.

FIGS. 20A-20C. The design and characterization of the triangular prism with the knotted DNA nanostructure (crossing number=22). FIG. 20A shows the design schematic that includes the folding pathway. FIG. 20B shows the sequence design parameters that facilitate the folding order. FIG. 20C shows representative AFM images of the self-assembled knotted NA nanostructures.

FIGS. 21A-21C. Design and characterization of a pentagonal pyramid that has a knotted DNA nanostructure (crossing number=25). FIG. 21A shows the design schematics of the folding pathway. FIG. 21B shows the sequence design parameters that facilitate the folding order. FIG. 21C shows representative AFM images of the self-assembled knotted NA nanostructures.

FIGS. 22A-22D. Cryo-EM characterization of a single stranded DNA tetrahedron. FIG. 22A shows a raw cryo-EM micrograph with visible nanostructures. FIG. 22B show the reference-free 2D class averages and projections of the final 3D reconstruction. FIG. 22C is a graph depicting a gold-standard FSC plot for the final 3D reconstruction of the nanostructures. FIG. 22D shows the tilt-pair validation result. The grey circle shows nanostructure pairs that cluster around the experimental tilt geometry.

FIGS. 23A-23C. Cryo-EM characterization of an ssDNA triangular prism (FIG. 23A), pyramid (FIG. 23B) and pentagonal pyramid (FIG. 23C). For each structure, a raw cryo-EM micrograph is shown with visible particles. There are also images on the right in smaller boxes, showing the raw particles. Furthermore, a Gold-standard FSC plot is also shown for the 3D reconstruction of these particles.

FIG. 24 shows one embodiment of a ssRNA knotted nanostructure in the form of a tetrahedron with crossing numbers.

FIG. 25 shows AFM images of ssRNA knotted nanostructures in the form of tetrahedrons with crossing numbers.

FIG. 26 shows ssRNA knotted nanostructures in the form of tetrahedron without crossing numbers.

FIG. 27 shows AFM images of ssRNA knotted nanostructures in the form of tetrahedrons without crossing numbers.

FIG. 28 shows DNA as a nanoscale building block.

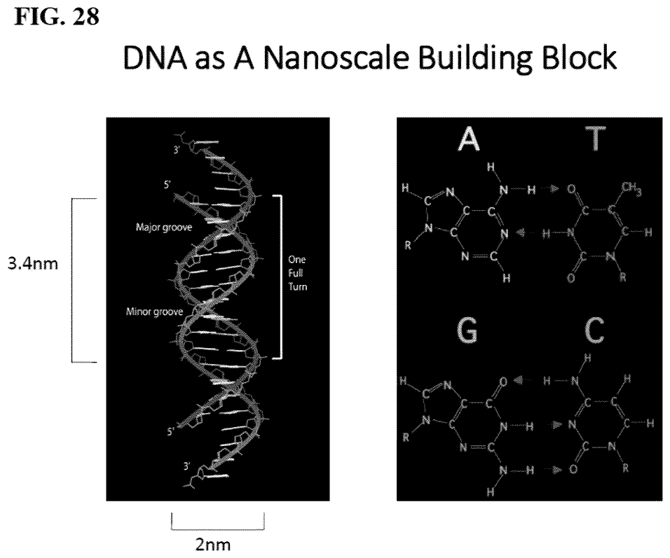

FIG. 29 shows two methods for creating DNA knotted nanostructures.

FIG. 30 shows single stranded nucleic acids: traditional DNA nanostructures and self-replicating ssDNA nanostructures.

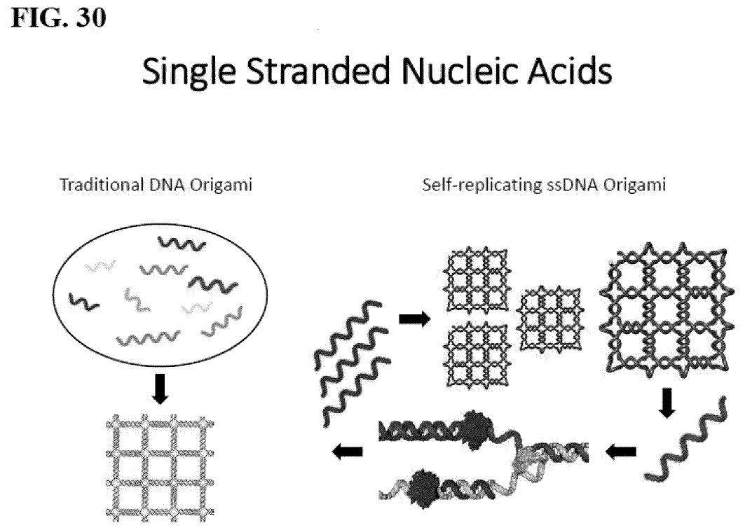

FIG. 31 shows knotted nanostructures with high numbers of crossings.

FIGS. 32A-32B shows a depiction of 3D knots.

FIG. 33 shows the size of nucleic acid knotted nanostrucutres of some embodiments of this disclosure.

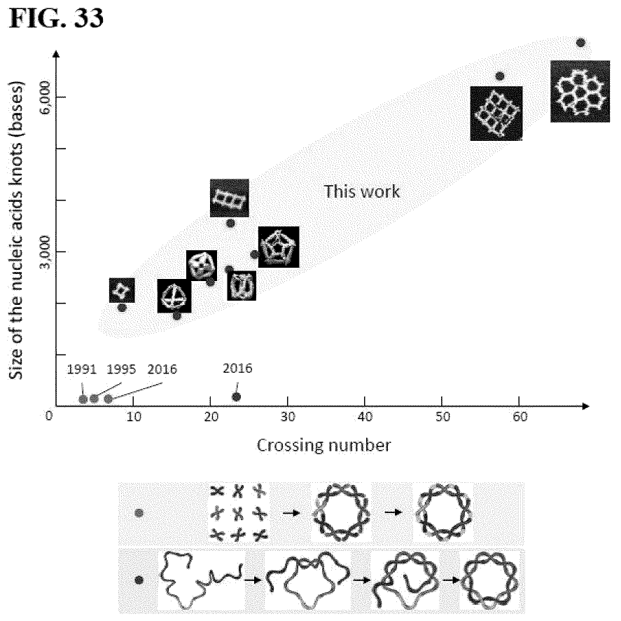

FIG. 34 depicts the production of ssRNA which creates knotted ssRNA nanostructures of some embodiments of this disclosure.

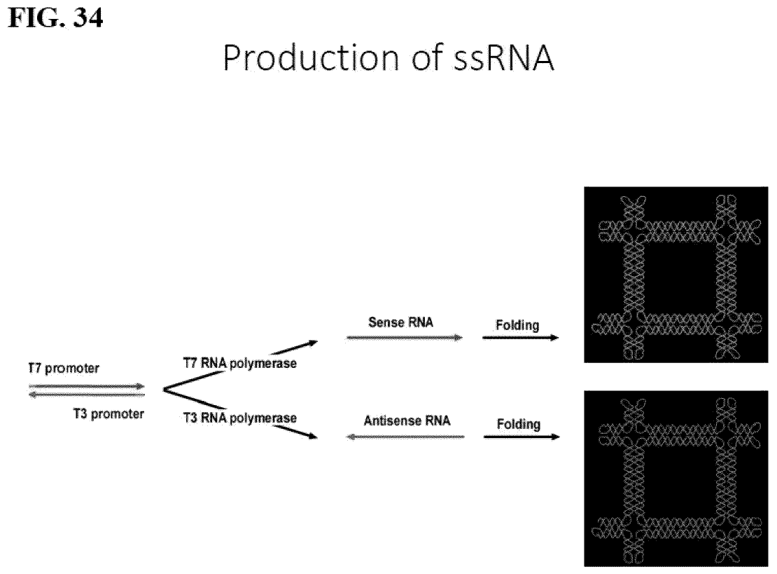

### DETAILED DESCRIPTION

This disclosure provides a method to create self-assembled ssDNA/ssRNA knotted nanostructures with high crossing numbers. These knotted nanostructures are self-assembled from a replicable single stranded nucleic acid and the knotted nanostructures are well organized with relative high yields. Various two-dimensional (2D) ssDNA knotted nanostructures are designed and characterized with high crossing numbers. The self-assembly is optimized with hierarchical folding, which significantly increases the yield. In some embodiments, the knotted nanostructure method creates ssRNA and three-dimensional (3D) ssDNA knotted nanostructures. In some embodiments, the crossover points occur in a periodic manner. This disclosure further provides for a method for constructing nucleic acids nanostructures with complex molecular topologies.

In some embodiments, this disclosure provides for a method of designing and constructing highly knotted nucleic acid nanostructures, each weaved from a single-stranded DNA and/or RNA chain by hierarchical folding in a prescribed order. In some embodiments, sets of DNA and/or RNA knotted nanostructures of two-or three-dimensional shapes are designed and constructed from NA sequences ranging from 1800 to 7500 nucleotides in length. The shapes of the nanostructures of this disclosure exhibit an unprecedented amount of complicated topological features, with high crossing numbers. Each step of the folding/threading process formed one crossing node and resulted in a surprisingly high yield of well-formed one crossing (˜96%). In some embodiments, the single-stranded DNA and/or RNA knots are replicated and amplified enzymatically in vitro or in vivo, and offers many potential transformative applications. In some embodiments, the ssDNA and/or ssRNA knotted nanostructures with high crossing numbers are self-assembled from a replicable single stranded nucleic acid and are well organized with relatively high folding yields compared to randomly designed NA sequences.

Complex molecular knots with high crossing numbers are achieved by folding, following a prescribed folding order, single-stranded DNA and/or RNA of customized sequences into target shapes.

### Programming Highly Knotted Molecular Topologies from Single-Stranded Nucleic Acids

Molecular knots represent some of the most extraordinary topological structures. Knotted DNA structures are often formed during genomic DNA replication and transcription. However, natural DNA knots are not predictable and do not have well-formed geometric shapes, nor periodic crossover events. The inventors have designed rules for programming synthetic nucleic acid sequences which self-assemble into highly knotted nanostructures with well-defined geometries and topologies.

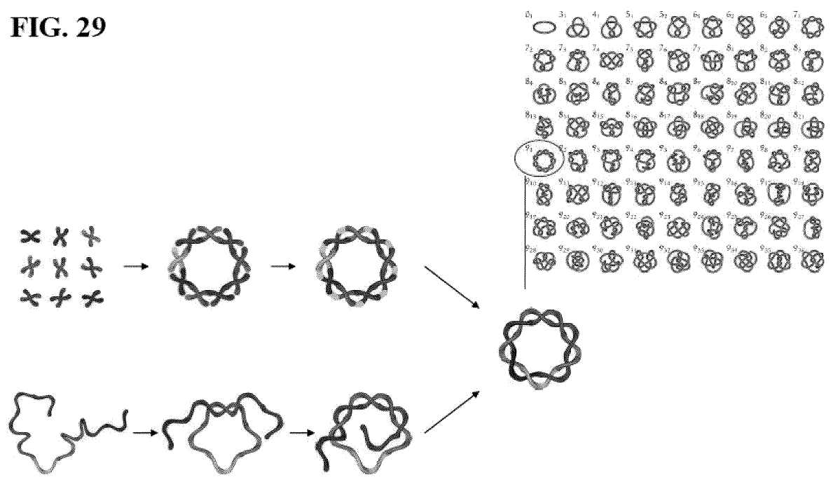

In some embodiments, the nucleic acid (DNA and/or RNA) knotted nanostructure is constructed by a “granular build-up method.” In some embodiments of the granular build-up method, as summarized in FIG. 29, a knot is assembled by connecting nine right-handed X-shaped junction tiles together. In some embodiments, the nucleic acid knot is constructed by a “single threading method.” In some embodiments of the the singular threading method, a single chain polynucleotide sequence is threaded through itself nine times. This disclosure includes a method for creating topological knots of single-stranded DNA and/or RNA with high crossing numbers. In some embodiments, the method comprises the use of single-stranded nucleic acids and high crossing numbers periodically throughout the nanostructure.

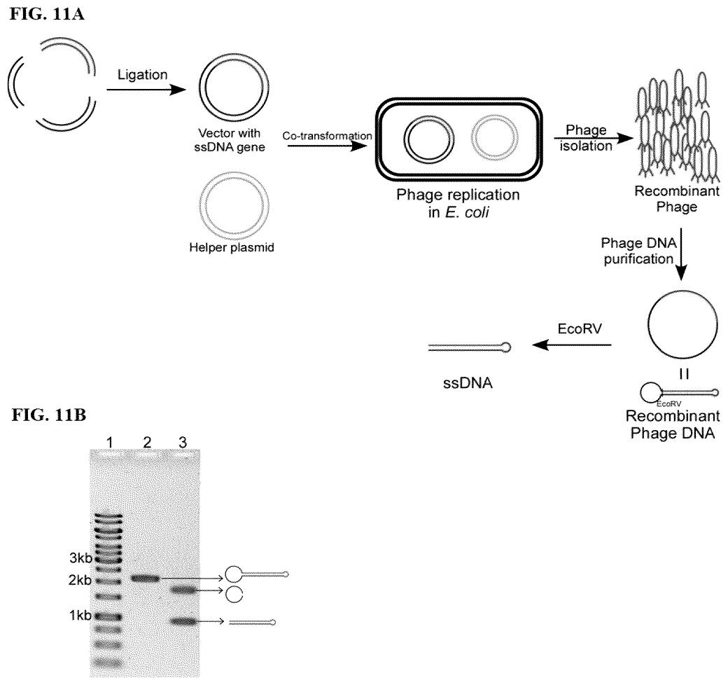

Both multi-stranded DNA and/or RNA sequences designed for NA knotted nanostructures and single-stranded DNA and/or RNA sequences designed for NA knotted nanostructures self-assemble via self-folding. There are advantages to the nucleic acids being single-stranded nucleic acids. As shown in FIG. 11, a ssDNA knotted nanostructure can be a self-replicating system, which enables cost-efficient, large-scale production using enzymatic and biological replication. The ssDNA knotted nanostructure also involves a unimolecular folding process which is independent of the reactant concentration, and offers higher folding yield and more robust folding kinetics than multi-stranded structures produced via concentration-dependent intermolecular self-assembly methods.

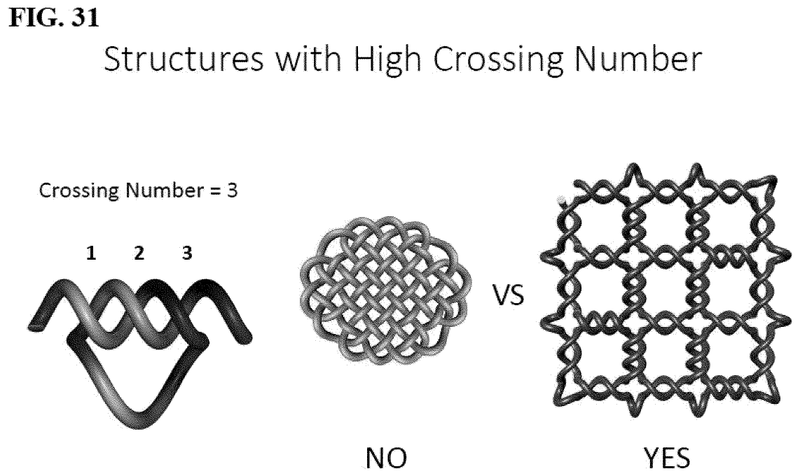

As used herein, the term “crossing” refers to a knot invariant that shows the smallest number of crossings in any diagram of the knot, representing the topological complexity of a knot. In some embodiments, the knots described herein can comprise 2 to 100 crossings. In some embodiments, the knots described herein can comprise from 9 to 67 crossings. In some embodiments, the crossing number of the knots described herein can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 crossings. In one embodiment, as shown in the left panel of FIG. 31, a knot consists essentially of a crossing number of 3. Compact structures do not facilitate the correct folding of DNA. To create high crossing number knots, “wireframe” networks are better candidates for constructing knotted structures because they offer more space for DNA chains to thread through the structures. A wireframe network is defined by the construction of 2D or 3D structures in which only vertices and edges/lines are used to define the outline of the structures. For DNA wireframe networks, the vertices are represented by multi-arm junctions and the edges are DNA tiles which can include or exclude: dsDNA helices, DX (double crossover) tiles, paranemic coheson tiles, and multi-helical bundles.

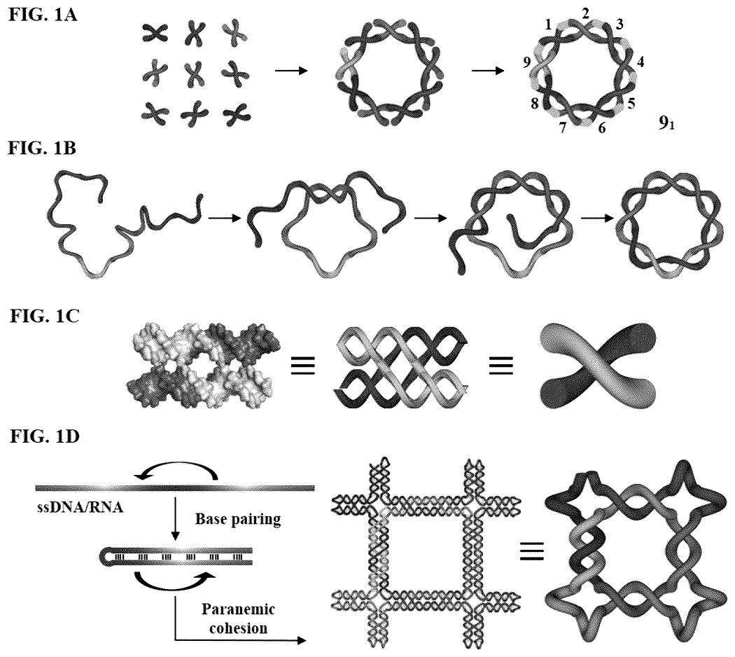

In some embodiments, the method for creating knotted NA nanostructures comprises using crossover motifs as the modular building blocks and a node-edge network as the geometric blueprint for arbitrary nanostructures. In some embodiments, as shown in, FIGS. 1A-1D two parallel crossovers are separated by 4 or 6 base pairs to form an X-shape. The nine X-shaped DNA tiles were arranged into a square with 2, 2, 2, and 3 crossings on the four edges respectively, and the adjacent paranemic cohesion junctions separated by 1 or 2 turns of the double stranded DNA. After connecting the nearest DNA strands and adding small linking structures at the four vertexes, the resulting design consisted of only one long contiguous ssDNA. The overall routing of the ssDNA could be treated as a two-step process. First, half of the DNA chain folded back to partially pair with the other half of the DNA, leaving several unpaired single-stranded regions in between the perfectly paired regions. Then, the unpaired regions matched to each other by paranemic cohesive interactions and finally knotted into the target topology.

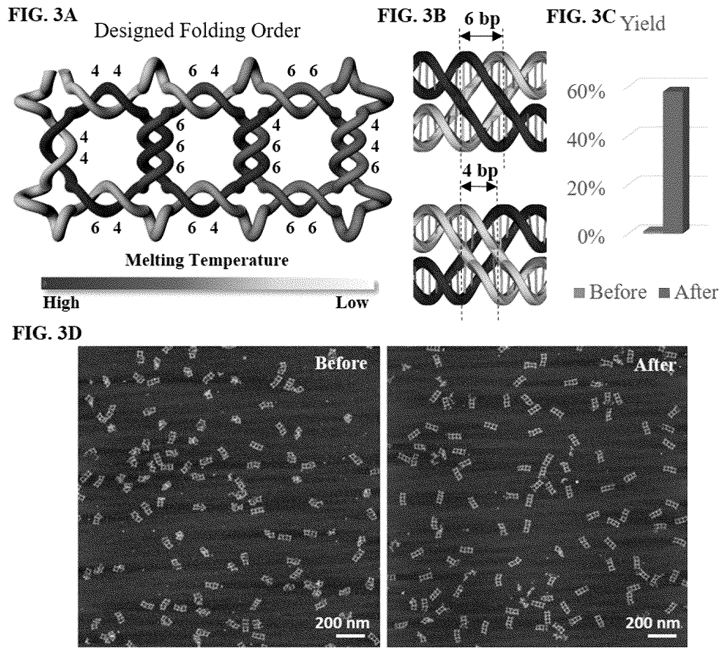

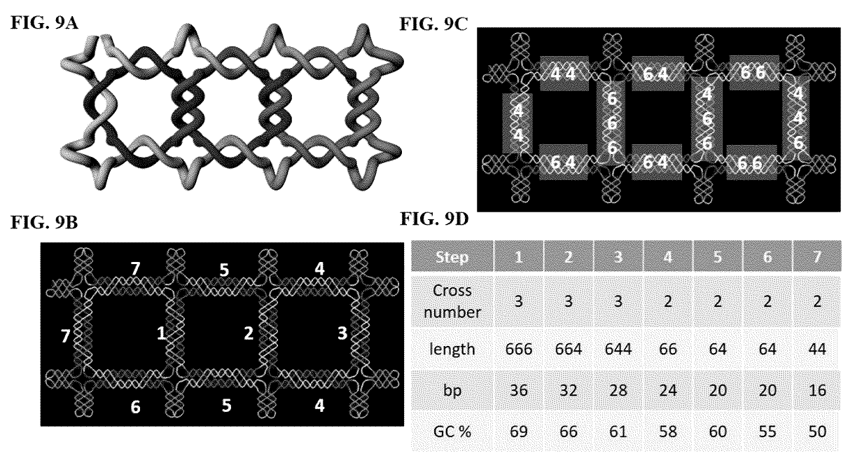

In some embodiments, this disclosure includes a topologically-and kinetically-favorable folding method to create knotted DNA and/or RNA nanostructures with high crossing numbers. In some embodiments, the crossings occur in a periodic manner. In some embodiments, the method involves a hierarchical folding strategy to guide the knotting process in a prescribed order. In some embodiments, as discussed in Example 1, the method comprises several essential rules for optimizing the folding pathway. The optimized folding path for a three-column grid structure with 23 crossings is shown in FIGS. 3A, 3B, and 9D. As shown in the foregoing figures, the folding order of the edges is guided and controlled by changing the number (2 or 3), the length (6 or 4 bp), and the sequence (GC percentage) of paranamic cohesion regions. The folding yield of the two types of scaffold routings were compared by AFM imaging as shown in FIGS. 3C, and 3D. The selected best folding pathway as shown in FIGS. 3A, 3B, 9D produced about 60% well-formed structures, while a control random other folding pathway showed a yield as low as 1%.

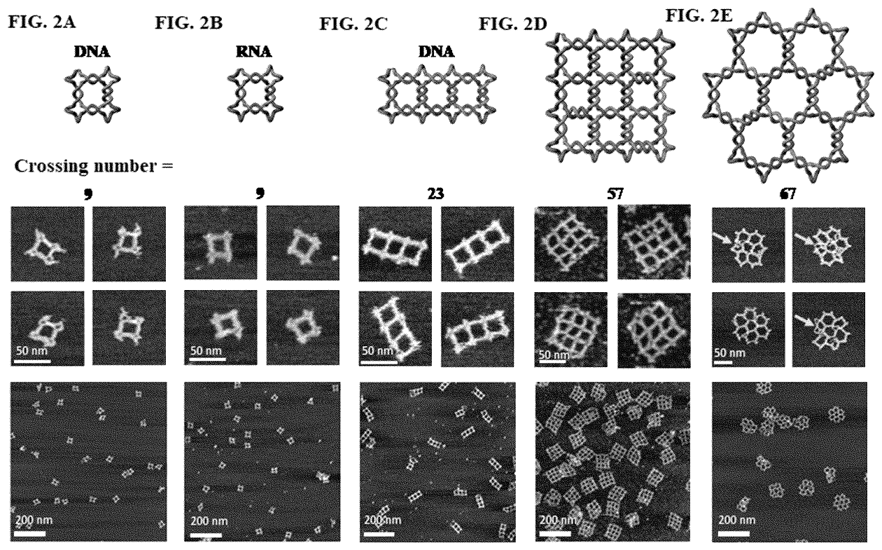

In some embodiments, the method comprises design procedures to create highly complex DNA knots with increasing crossing numbers occurring in a periodic manner. In some embodiments, as shown in FIGS. 2A-2E, a 3 by 3 square grid of DNA knots with 57 crossed nodes in a periodic manner was designed with an optimized folding pathway. Other geometric layouts also follow similar design principles. A large molecular knot with 67 crossings in a hexagonal lattice was also designed and constructed. In some embodiments, the design strategies for ssDNA knotted nanostructures is adapted to create ssRNA knots by adjusting selected design parameters and experimental conditions as described herein. Each of these ssDNA and/or ssRNA knots are folded from a replicable single-stranded nucleic acid molecule with sizes ranging from 1800 to 7500 nucleotides. High-resolution AFM images confirm the successful formation of the target nanostructures, as shown in FIGS. 2A-2E.

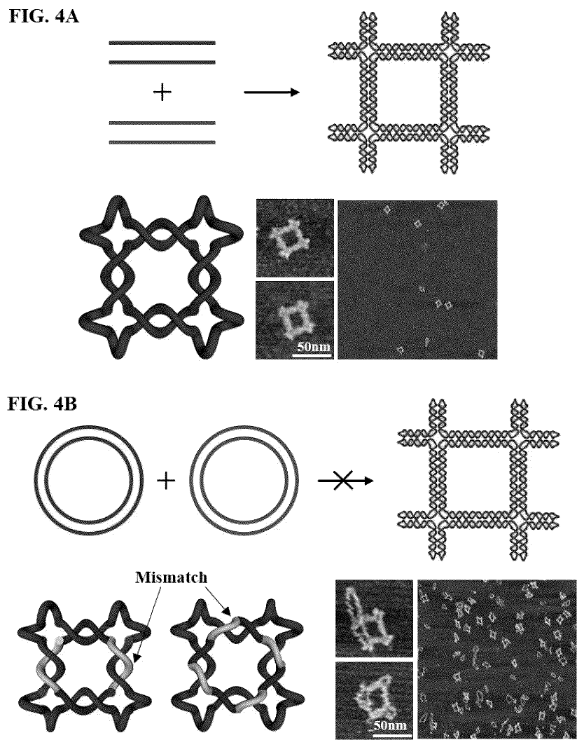

AFM imaging confirms the formation of the defected nodes in the final knotted NA nanostructure comprising perfect-match or mis-matched links. Topological control experiments were conducted to validate the use of AFM imaging as a method to detect the formation of defected nodes. As shown in FIGS. 4A-4B, a link structure was designed with a linking number of 8 as the topological control. As used herein, the term “linking number” refers to the number of links within a knotted NA nanostructure. A link is a sub-structure comprising two dsDNA rings, which connected to each other through 8 paranemic cohesions. This particular link structure was constructed by annealing two linear dsDNAs with 8 unpaired bubbles. These unpaired regions within these two linear dsDNAs interact with their counterparts to form the stable paranemic cohesions. Sticky ends closed the two rings after the formation of the nanostructure. This particular link structure assembled well as compact conformations, and the nanostructures were characterized by high resolution AFM images, as shown in FIGS. 4A-4B. If, however, the two linear dsDNAs were first ligated to form closed rings before interaction, these two dsDNA rings would not be able to interweave into the fully inter-locked loop structure. As expected, although the two dsDNA rings could still bind with each other partially through some of the paranemic cohesion interactions, extensive defects were observed in all of the structures under high resolution AFM. This experiment confirmed that the AFM imaging method can confirm the formation of the inter-lock topology.

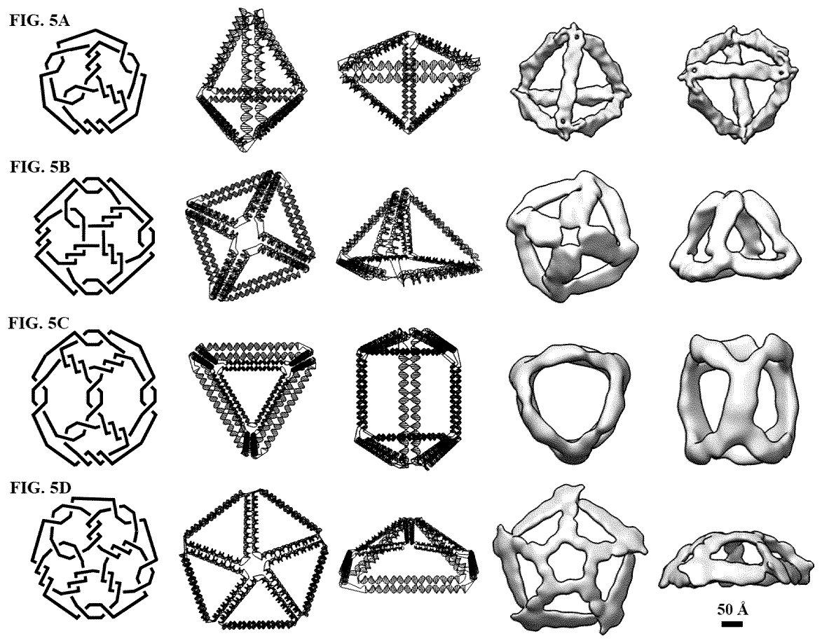

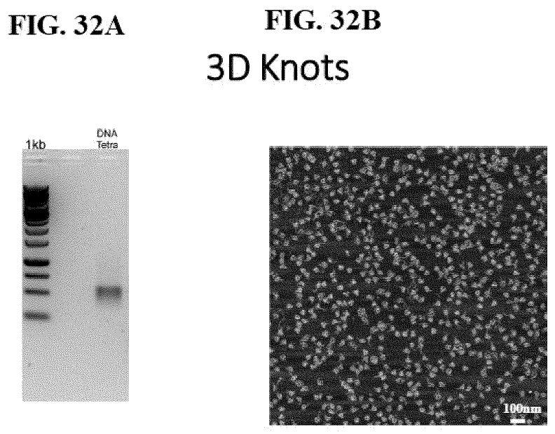

In some embodiments, this disclosure provides for a method to create 3D architectures with arbitrary geometries comprising knotted NA nanostructures as described herein. The versatility of the method was demonstrated by constructing four ssDNA polyhedral meshes as shown in FIGS. 5A-5D: a tetrahedron, a pyramid, a triangular prism, and a pentagonal pyramid with crossing numbers 15, 20, 22, and 25, respectively. A schlegel diagram transfers the 3D objects to their topologically equivalent 2D nets. Optimized folding pathways were designed carefully for step-wise hierarchical assembly and the corresponding ssDNA strands were designed, synthesized, and assembled. AFM images showed an abundance of well-folded 3D nanoparticles with the expected sizes. Single particle cryo-EM 3D reconstruction revealed that the overall conformations matched the designed geometries well. The vertex design for the ssDNA knots was different from the multi-arm junction design based on the double crossover motif, as shown in FIGS. 5A, 18B, 32A, and 32B. There is a 5 bp difference in length between the two parallel dsDNAs that form each edge, leading to chiral vertices and inclined edges in the ssDNA knots. This unique geometric feature identifies conformational diastereomers, which is when the structure is turned inside out, while satisfying all programmed Watson-Crick base pairing with the same network. The 3D reconstruction data indicated that the ssDNA knots preferred to point the major grooves inwards at the vertices.

It has been a long-standing challenge to construct molecular knots with increasing size and complexity in a programmable and controllable way. In some embodiments, this disclosure provides for methods of folding single-stranded nucleic acids with completely custom-designed sequences, to create ssDNA and/or ssRNA nanostructures with highly complex topologies that are programmable, replicable, and scalable. This disclosure further provides for various 2D nanostructures which are designed, constructed, and characterized using high resolution AFM imaging. The folding yield of the knots is optimized by following a set of design rules to select the best folding pathway and programming the step-wise hierarchical folding pathway through sequence design in the paranemic cohesion regions. In some embodiments, the method is applied to construct ssRNA knotted nanostructures and/or three-dimensional (3D) ssDNA knotted nanostructures. The 3D knotted nanostructures are characterized by cryogenic transmission electronic microscopy (cryo-EM) single particle reconstruction, which confirmed that the nanostructures had assumed the designated geometry. The present ssDNA knotted nanostructures are much larger than structures made by other methods (up to 7.5k bases, compared to only up to 30 bases for other methods) and can exhibit complex topology structures (with as high as 67 crossing numbers, compared to only 1 crossing for other methods).

### Design Principles for ssRNA Knotted Nanostructures

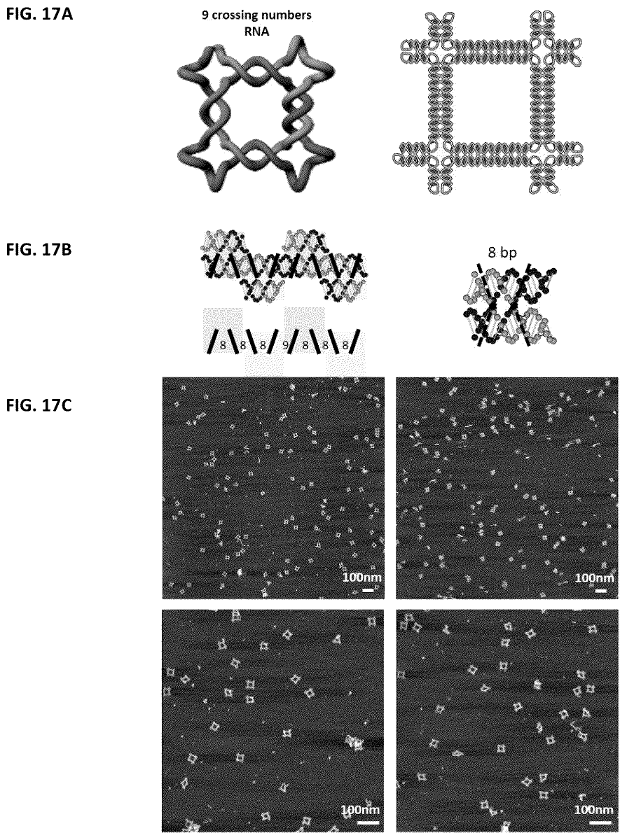

In some embodiments, this disclosure provides for methods of designing and creating ssRNA knotted nanostructures. An X-shaped RNA modular building block is designed that was similar to the paranemic cohesion structures of DNA. The same steps are followed for constructing the ssRNA knotted nanostructures as the steps for constructing ssDNA knotted nanostructures. First, based on the 3D modeling of an A-form dsRNA helix (11 bp per helical turn, 19 degree inclination of base pairs) and the best geometric fitting, 8 (instead of 4 or 6) base pairs were chosen for the length of a paranemic crossover, as shown in FIG. 17B. For an 8 base-pair paranemic cohesion, a total of 4⁸=65536 possible sequences provided an adequate sequence space for the selection of unique complementarity to sufficiently avoid undesired interactions between the paranemic cohesion motifs. Second, given the 11 base pairs per turn of an A-form dsRNA, the lengths of the inter-motif stems were assigned as alternating between 8 and 9 bp, as shown in FIGS. 17A-17C) to achieve a structural repeating unit of 33 bps for three full helical turns (i.e. 8 bp+8 bp stem+8 bp+9 bp stem=33 bp=3 full turns). In this design, the neighboring structural units were in line with each other without accumulating helical twist, and the final assembled knotted NA nanostructure was expected to stay in 2D. Like the ssDNA 9₁ knot, 2, 2, 2, and 3 crossings were assigned on the four edges of a square, respectively, and looped the vertices to form one single-stranded RNA. After generating the appropriate sequences by following the same sequence design rules as the ssDNA nanostructures, the dsDNA gene coding for the long RNA strand was synthesized, and the ssRNA molecule was obtained by an in vitro transcription reaction. After an annealing step, the ssRNA knotted nanostructures comprising 9₁ knots were confirmed by AFM images.

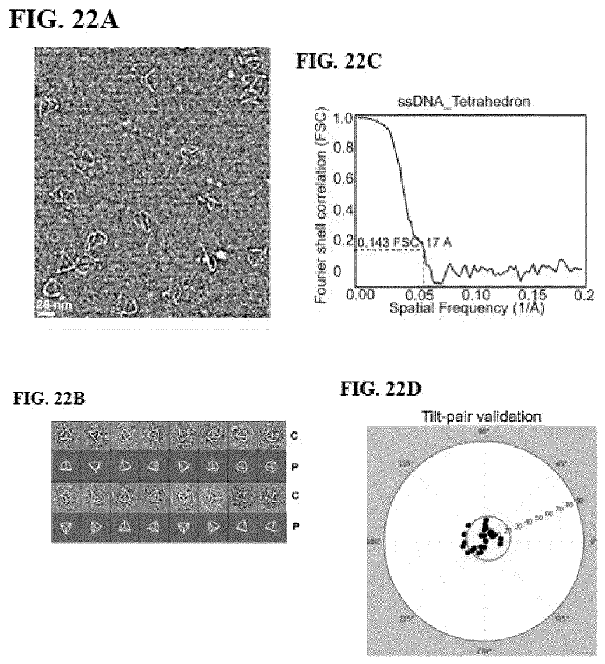

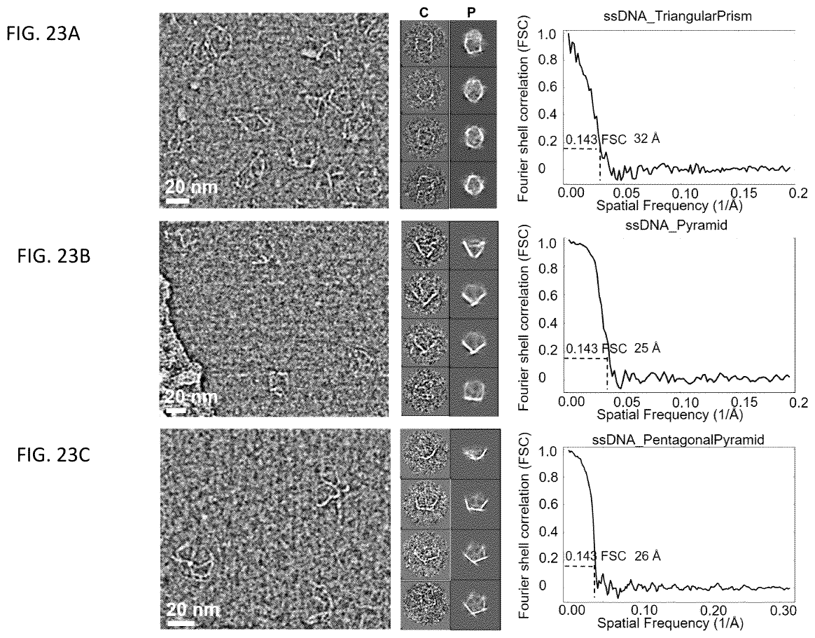

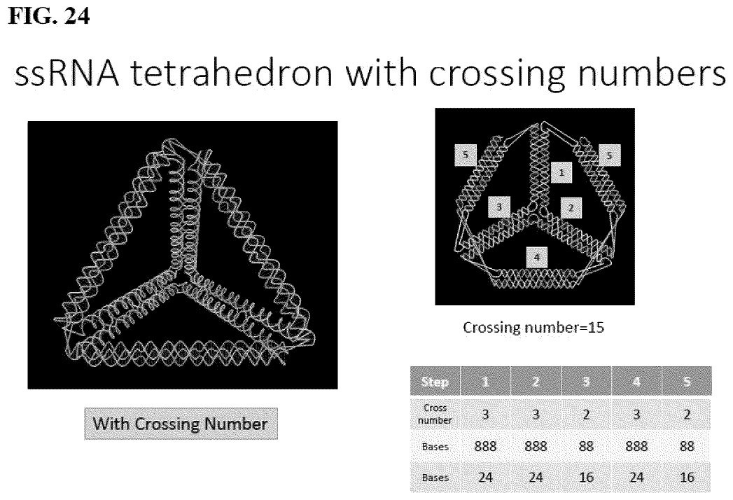

Both the chemical and enzymatic synthesis of long ssDNA molecules is technically challenging because the chain possesses a large portion of self-complementarity. As shown in the folding pathway depicted in FIG. 11A, the ssDNA molecule first forms a long hairpin structure with the 5′ and 3′ ends meeting each other. The full-length ssDNA strand was first split into two equal halves, with each strand lacking significant secondary structures. Each of the halves were then inserted into plasmids as double stranded genes and amplified by cloning. The two dsDNA genes were obtained separately from the plasmids by restriction enzymes digestion (EcoRI+XbaI and XbaI+HindIII respectively) and were then ligated together with a linearized phagemid vector, pGEM-7zf(−). In order to obtain the full-length ssDNA molecule, the recombinant M13 phage was replicated in E. coli with the assistance of a helper plasmid, pSB4423. Because the helper plasmid, pSB4423, does not contain a phage replication origin, only the phagemid vector containing the full-length ssDNA gene was able to act as a template for the phage DNA replication. After the extraction and purification of the recombinant phage DNA, EcoRV digestion was performed to cut out the target ssDNA. Native agarose gel electrophoresis was used to separate the target ssDNA from the phagemid vector ssDNA. Using this method, all of the long ssDNA strands were synthesized and amplified at a nanomole quantity (with 1 L scale of E. coli culture) and high purity. The ssDNA strand was obtained, then self-assembled (folded) in a 1×TAE-Mg buffer with a 12 hour or 24 hour annealing ramp from 65° C. to 25° C., as shown in FIGS. 22A-22D and 23A-23C. The folded products were then characterized by using AFM imaging, gel electrophoresis and/or cryo-EM imaging. ssRNA knotted nanostructures were similarly produced, as shown in FIG. 24.

### Nucleic Acid Knotted Nanostructures and Compositions Thereof

As used herein, the term “nucleic acid nanostructure” refers to a nanoscale structure comprising nucleic acid (NA), wherein the nucleic acid acts both as a structural and function clement. In some embodiments, the nucleic acid nanostructure is DNA. In some embodiments, the nucleic acid nanostructure is RNA. In some embodiments, the nucleic acid nanostructure comprises both DNA and RNA. In some embodiments, NA nanostructures can also serve as a scaffold for the formation of other structures. The nucleic acid nanostructures are prepared based on the concept of base-pairing. While no specific sequence is required, the sequences of each oligonucleotide segment must be partially complementary to certain other sequences within the oligonucleotide segment to enable hybridization and assembly of the nanostructure. In certain embodiments, the nucleic acid nanostructure is a nucleic acid rectangle nanostructure, self-assembled from one single-stranded nucleic acid molecule through paranemic cohesion crossover.

In some embodiments, the length of each nucleic acid strand is variable and depends on the type of nanostructure to be created. In some embodiments, the nucleic acid nanostructure is comprised of a single oligonucleotide strand. In some embodiments, the NA strand is about 100 nucleotides in length to about 20,000 nucleotides in length, or any length there-between. In some embodiments, the NA strand is about 100 nucleotides in length to about 10,000 nucleotides in length, 1000 nucleotides in length to about 10,000 nucleotides in length, 1200 nucleotides in length to about 10,000 nucleotides in length, about 1200 to about 7500 nucleotides in length, about 1500 to about 8000 nucleotides in length, about 1800 to about 7500 nucleotides in length. In some embodiments, the nucleic acid strand is about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, or about 10000 nucleotides in length, or between any of the aforementioned lengths.

In some embodiments, the NAs are synthesized de novo using oligonucleotide synthesis methods. In some embodiments, the oligonucleotide synthesis methods are selected from the cyanoethyl phosphoramidite method (Beaucage, S. L., and Caruthers, M. H., Tet. Let. 22:1859,1981); nucleoside H-phosphonate method (Garegg et al., Tet. Let. 27:4051-4054,1986; Frochler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market, including the use of an in vitro transcription method. In some embodiments, the oligonucleotide synthesis methods are selected from enzymatic step-wise addition methods. The enzymatic step-wise addition methods can include or exclude the method described in: WO 2016/034807, WO 2015/159023, WO 2019/030149, US 2014/0363852, US 2018/0274001, and US 2016/0108382, each of which are herein incorporated by reference.

In some embodiments, the NA nanostructure exhibits increased nuclease resistance compared to a control. In some embodiments, the control is an unfolded single-stranded nucleic acid molecule comprising the same nucleic acid sequence as the NA nanostructure. In some embodiments, nuclease resistance of the NA nanostructure is 10%, 20%, 30%, 40%, 50%, 60%, 70%, 80%, 90%, 95% or more than a control NA sequence.

In some embodiments, the NA nanostructure is assembled using a single stranded NA molecule. In some embodiments, the NA nanostructure comprises both single stranded and double stranded regions. In some embodiments, the single-stranded regions are at the 3′ terminus, the 5′ terminus, or both the 3′-and 5′-terminus of the nanostructure sequence.

In some embodiments, the NA nanostructure is comprised of one ssNA molecule that self-assembles into a nanostructure. In some embodiments, the NA nanostructure is assembled from one ssNA molecule through paranemic cohesion crossovers. As used herein, the term “paranemic cohesion crossover” refers to a four-stranded nucleic acid complex containing a central dyad axis that relates two flanking parallel double helices. In some embodiments, paranemic cohesion crossovers form when bases outside a hairpin structure pair with bases within the hairpin or internal loop. In some embodiments, the paranemic adhesion crossovers occur in a periodic manner throughout the nanostructure. The periodicity can be a single phase or multiple phases. In some embodiments, the number of multiple phases of periodicity are 2, 3, 4, 5, 6, 7, 8, 9, or 10. In some embodiments, the length of each phase is independently selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or more base pairs.

In certain embodiments, the NA nanostructure comprises one single-stranded NA (ssNA) molecule, wherein the ssNA molecule forms at least one paranemic cohesion crossover. In some embodiments the NA nanostructure is a rectangle nanostructure. In some embodiments, the single stranded NA molecule is selected from any one of SEQ ID NO:1-15.

In some embodiments, this disclosure provides for a NA nanostructure comprising a nucleic acid sequence having at least about 60% sequence identity to any one of SEQ ID NO:1-15. In some embodiments, the NA nanostructure comprises a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:1-15. In some embodiments, the NA nanostructure consists of a nucleic acid sequence having at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:1-15. In certain embodiments, the NA nanostructure comprises any sequence comprising any one of SEQ ID NO:1-15. In some embodiments, the NA nanostructure consists of any one of SEQ ID NO:1-15.

In some embodiments, the NA nanostructure comprises one or more modified nucleic acids. In some embodiments, the one or more modified nucleic acids are selected from inosine residues, alkynyl-modified nucleotides,

In some embodiments, the alkynyl modified nucleotides are chemically synthesized from a phosphoramidite selected from: 5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N- diisopropyl)]-phosphoramidite, 5′-Dimethoxytrityl-5-ethynyl-2′-deoxyUridine, 3′-[(2-cyanoethyl)-(N,N-diisopropyl)]-phosphoramidite, 5′-Hexynyl Phosphoramidite, 5′-Dimethoxytrityl-5-(octa-1,7-diynyl)-2′-deoxyuridine, 3′-[(2-cyanoethyl)-(N,N-diisopropyl)]-phosphoramidite, 10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite, 6-Bromo-hex-1-yl-(2-cyanoethyl)-(N,N-diisopropyl)-phosphoramidite, 3-Dimethoxytrityloxy-2-(3-(5-hexynamido) propanamido) propyl-1-O-[(2-cyanoethyl)-(N,N-diisopropyl)]-phosphoramidite, and 5′-Dimethoxytrityl-3′-propargyl-5-methyl-2′-deoxyCytosine-N-succinoyl-long chain alkylamino-CPG.

In some embodiments, one or more agents (e.g., 1, 2, 3, 4, 5, 6, 7, 8, 9, 10 etc.) are operably linked to the NA nanostructure. The one or more agents can include or exclude diagnostic agents or therapeutic agents. In some embodiments, at least one diagnostic agent is operably linked to the NA nanostructure. In some embodiments, at least one therapeutic agent is operably linked to the NA nanostructure. In some embodiments, at least one diagnostic agent and at least one therapeutic agent are operably linked to the NA nanostructure.

Diagnostic agents can include or exclude fluorophores, radioisotopes, nanoparticles, and colorimetric indicators.

As used herein, the term “therapeutic agent” refers to agents that provide a therapeutically desirable effect when administered to an animal. The animal is a mammal, which can include or exclude a human. The therapeutic agent may be of natural or synthetic origin. In some embodiments, the therapeutic agent can include or exclude a nucleic acid, a polypeptide, a protein, a peptide, a radioisotope, saccharide or polysaccharide or an organic compound, which can include or exclude a small molecule. The term “small molecule” includes organic molecules having a molecular weight of less than about, e.g., 1000 daltons. In one embodiment a small molecule can have a molecular weight of less than about 800 daltons. In another embodiment a small molecule can have a molecular weight of less than about 500 daltons.

In some embodiments, the therapeutic agent is an immuno-stimulatory agent, a radioisotope, a chemotherapeutic drug or an immuno-therapy agent. In some embodiments, the immune-stimulatory agent can include or exclude an antibody or an antibody fragment. In some embodiments, the therapeutic agent is a vaccine. In some embodiments, the vaccine can include or exclude a cancer vaccine. In some embodiments, the therapeutic agent is a tumor targeting agent. In some embodiments, the tumor targeting agent is selected from a monoclonal tumor-specific antibody, a nanobody, a scFv, or an aptamer. In some embodiments, the therapeutic agent is an antibody. In some embodiments, the antibody therapeutic agent is a monoclonal antibody. In some embodiments, the monoclonal antibody is an anti-PDI antibody. In some embodiments, the therapeutic agent is an antigen. In some embodiments, the antigen is selected from a tumor associated antigen or a tumor specific antigen. In some embodiments, the therapeutic agent is a tumor antigen peptide(s).

The linkage between the agent(s) and the NA nanostructure can include any group that connects the NA nanostructure and the agent, provided that said linkage does not interfere with the function of the agent or the NA nanostructure. In some embodiments, chemistries that link the agent to an oligonucleotide can include or exclude disulfide linkages, amino linkages, and covalent linkages. In some embodiments, the linker can include or exclude aliphatic or ethylene glycol linkers. In some embodiments, the linker can include or exclude phosphodiester, phosphorothioate and/or other modified linkages. In some embodiments, the linker is a binding pair. In some embodiments, the “binding pair” refers to two molecules which interact with each other through any of a variety of molecular forces including or excluding ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate probe, IgG-protein A, antigen-antibody, aptamer-target and the like. In some embodiments, a first member of the binding pair comprises avidin or streptavidin and a second member of the binding pair comprises biotin.

### Compositions and Kits

Certain embodiments of the invention also provide a composition comprising an NA nanostructure described herein and a carrier. In some embodiments, the composition comprises a plurality of NA nanostructures.

In some embodiments, the composition further comprises at least one therapeutic agent described herein.

In some embodiments, the composition is pharmaceutical composition and the carrier is a pharmaceutically acceptable carrier.

The present invention further provides kits for practicing the present methods. Certain embodiments of the invention provide a kit comprising an NA nanostructure described herein and instructions for administering the NA nanostructure to induce an immune response or to treat a disease or condition. In some embodiments, the immune response is anti-tumor immunity. In some embodiments, the kit further comprises a therapeutic agent described herein and instructions for administering the therapeutic agent in combination (simultaneously or sequentially) with the NA nanostructure.

### Certain Methods

In some embodiments, an NA nanostructure described herein is used as an

immuno-adjuvant to boost an immune response. In some embodiments, the immune response induces anti-tumor immunity.

Certain embodiments of the invention provide a method of inducing an immune response a subject. The subject is a mammal, which can include or exclude a human. The method comprises administering to the subject an effective amount of an NA knotted nanostructure or composition as described herein.

In some embodiments, the administration of the NA knotted nanostructure described herein increases an immune response by at least about, e.g., 20%, 30%, 40%, 50%, 60%, 70%, 80%, 90%, or more, compared to a control. Methods of measuring an immune response include using an assay as described in the Examples. As used herein, the phrase “inducing an immune response” refers to the activation of an immune cell. Methods of measuring an immune response include using an assay described in the Examples. As used herein, the phrase “effective amount” refers to an amount of an NA nanostructure described herein that induces an immune response.

Certain embodiments of the invention also provide a method of treating a disease or disorder in a subject, comprising administering to the subject a therapeutically effective amount of an NA nanostructure or a composition as described herein.

In some embodiments, a method of the invention further comprises administering at least one therapeutic agent to the subject.

In some embodiments, the at least one therapeutic agent is administered in combination with the NA nanostructure. As used herein, the phrase “in combination” refers to the simultaneous or sequential administration of the NA nanostructure and the at least one therapeutic agent. For simultaneous administration, the NA nanostructure and the at least one therapeutic agent is present in a single composition or is separate. In some embodiments, when the NA nanostructure and at least one thereapeutic agent are administered simultaneously, they are administered by either the same or different routes.

Certain embodiments of the invention provide an NA nanostructure or a composition as described herein for use in medical therapy.

Certain embodiments of the invention provide the use of an NA nanostructure or a composition as described herein for the manufacture of a medicament for inducing an immune response in a subject. In some embodiments, the subject is a mammal, which can include or exclude a human.

Certain embodiments of the invention provide the use of an NA nanostructure or a composition as described herein for the manufacture of a medicament for inducing an immune response in a subject in combination with at least one therapeutic agent. In some embodiments, the subject is a mammal, which can include or exclude a human.

Certain embodiments of the invention provide an NA nanostructure or a composition as described herein for inducing an immune response.

Certain embodiments of the invention provide an NA nanostructure or a composition as described herein for inducing an immune response, in combination with at least one therapeutic agent.

Certain embodiments of the invention provide the use of an NA nanostructure or a composition as described herein for the manufacture of a medicament for treating a disease or disorder in a subject.

Certain embodiments of the invention provide the use of an NA nanostructure or a composition as described herein for the manufacture of a medicament for treating a disease or disorder in a subject, in combination with at least one therapeutic agent.

Certain embodiments of the invention provide an NA nanostructure or a composition as described herein for the prophylactic or therapeutic treatment a disease or disorder.

Certain embodiments of the invention provide an NA nanostructure or a composition as described herein for the prophylactic or therapeutic treatment of a disease or disorder, in combination with at least one therapeutic agent.

In some embodiments, the disease or disorder is a condition that requires a boost of the host immunity. In some embodiments, the disease or disorder is cancer. In some embodiments, the disease or disorder is an infectious disease.

In some embodiments, the cancer is carcinoma, lymphoma, blastoma, sarcoma, or leukemia. In some embodiments, the cancer is a solid tumor cancer.

In some embodiments, the cancer is squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (which can include or exclude endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia, or head and neck cancer. In some embodiments, the cancer is breast cancer.

In some embodiments, the therapeutic agent is a therapeutic agent described herein. In certain embodiments, the therapeutic agent is selected from an immuno-stimulatory agent, a radioisotope, a chemotherapeutic drug or an immuno-therapy agent. In some embodiments, the immuno-therapy agent can include or exclude an antibody or an antibody fragment. In some embodiments, the therapeutic agent is a vaccine, which can include or exclude a cancer vaccine. In some embodiments, the therapeutic agent is a tumor targeting agent, which can include or exclude a monoclonal tumor-specific antibody or an aptamer. In some embodiments, the therapeutic agent is an antibody. In some embodiments, the therapeutic agent is a monoclonal antibody. In some embodiments, the monoclonal antibody is an anti-PDI antibody. In some embodiments, the therapeutic agent is an antigen. The antigen is selected from a tumor associated antigen or a tumor specific antigen. In some embodiments, the therapeutic agent is a tumor antigen peptide(s).

### Administration

As described herein, methods of the invention comprise administering a composition comprising an NA nanostructure described herein, and optionally, a therapeutic agent to a subject. In some embodiments, such compositions are formulated as a pharmaceutical composition and administered to a mammalian host, which can include or exclude a human patient in a variety of forms adapted to the chosen route of administration, i.e., orally or parenterally, by intravenous, intramuscular, intraperitoneal or topical or subcutaneous routes.

In some embodiments, the compositions are systemically administered, e.g., orally, in combination with a pharmaceutically acceptable vehicle which can include or exclude an inert diluent or an assimilable edible carrier. The compositions may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained. The tablets, troches, pills, capsules, and the like may also contain the following: binders which can include or exclude gum tragacanth, acacia, corn starch or gelatin; excipients which can include or exclude dicalcium phosphate; a disintegrating agent which can include or exclude corn starch, potato starch, alginic acid and the like; a lubricant which can include or exclude magnesium stearate; and a sweetening agent which can include or exclude sucrose, fructose, lactose or aspartame or a flavoring agent which can include or exclude peppermint, oil of wintergreen, or cherry flavoring may be added. When the unit dosage form is a capsule, it may contain, in addition to materials of the above type, a liquid carrier, which can include or exclude a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring which can include or exclude cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. In some embodiments, the liquid carrier or vehicle can be a solvent or liquid dispersion medium comprising a liquid which can include or exclude: water, ethanol, a polyol (for example, glycerol, propylene glycol, liquid polyethylene glycols, and the like), vegetable oils, nontoxic glyceryl esters, and suitable mixtures thereof. The proper fluidity can be maintained by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids which can include or exclude talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants which can include or exclude fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners which can include or exclude synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver a compound to the skin can include or exclude: Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), each of which are herein incorporated by reference in their entirety.

Useful dosages of compounds can be determined by comparing their in vitro activity, and in vivo activity in animal models. Methods for the extrapolation of effective dosages in mice, and other animals, to humans can include U.S. Pat. No. 4,938,949, herein incorporated by reference.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound formulated in such a unit dosage form. The desired dose may conveniently be presented in a single dose or as divided doses administered at appropriate intervals. In some embodiments, the dose interval is selected from two, three, four or more sub-doses per day. The sub-dose itself may be further divided, e.g., into a number of discrete loosely spaced administrations; which can include or exclude multiple inhalations from an insufflator or by application of a plurality of drops into the eye.

### Nucleic Acid Nanostructure

In some embodiments, single-stranded nucleic acid (ssNA) nanostructure described herein is a component in a nanostructure and functions as a molecular payload carrier, including inducing anti-tumor vascularization effects.

In some embodiments, the ssNA nanostructure further comprises NA targeting strands, wherein each targeting strand is operably linked to a targeting moiety. In some embodiments, the targeting moiety is an aptamer. In some embodiments, the aptamer is specific for nucleolin.

In some embodiments, the ssNA nanostructure further comprises NA imaging strands, wherein each imaging strand is operably linked to an imaging agent. In some embodiments, the imaging agent is fluorescent dye.

As used herein, the term “ssNA nanostructure” refers to a nanoscale structure comprising a NA, wherein the NA acts both as a structural and functional element. In some embodiments, ssNA nanostructures also serve as a scaffold for the formation of other structures. In some embodiments, ssNA nanostructures are prepared from one or more nucleic acid oligonucleotides. In some embodiments, the ssNA nanostructure is an ssNA rectangle knotted nanostructure, self-assembled from single-stranded DNA or RNA molecules. In some embodiments, the ssNA nanostructure further comprises one or more fastener strands of DNA, wherein the one or more fastener strands of DNA fastens the rectangular sheet into a tube-shaped knotted NA nanostructure. In some embodiments, the rectangular sheet is about 10 to about 150 nm in length (e.g., 10, 20, 30, 40, 50, 60, 70, 80, 90, 100, 110, 120, 130, 140, or150 nm), and about 10 to about 150 nm width. In some embodiments, the dimension of the rectangular sheet is about 90 nm×about 60 nm×2 nm.

In some embodiments, the tube-shaped knotted NA nanostructure has a diameter of about about 10-50 nm. In some embodiments, the tube-shaped knotted NA nanostructure has a diameter of about about 19 nm.

In some embodiments, the ssNA nanostructures comprises one or more DNA capture strands, wherein each capture strand is operably linked to a therapeutic agent.

In some embodiments, the ssNA nanostructure further comprises NA targeting strands, wherein each targeting strand is operably linked to a targeting moiety. In some embodiments, the targeting moiety is an aptamer.

In some embodiments, the fastener strand is a Y-shaped structure. In some embodiments, the Y-shaped structure comprises an F50 AS1411 aptamer sequence that specifically binds to nucleolin, and a Comp15 DNA strand partially complementary to the AS1411 sequence, wherein the F50 and the Comp15 sequences form a 14-to 16-base pair duplex.

In some embodiments, the Y-shaped structure comprises F50 and Comp15; F50-48 and Comp15-48; F50-73 and Comp15-73; F50-97 and Comp15-97; F50-120 and Comp15-120; F50-144 and, Comp15-144; or F50-169 and Comp15-169. In some embodiments, the Y-shaped structure sequences can include or exclude a fluorophore or quencher at the 3′ or 5′ (or both) terminae of the sequences. In some embodiments, the fluorophore is FITC. In some embodiments, the quencher is BHQ.

In some embodiments, the fastener strands comprise a nucleic acid sequence comprising the sequence of:

F50:
(SEQ ID NO: 18)
5′-
GGTGGTGGTGGTTGTGGTGGTGGTGGTCTAAAGTTTTGTCGTGAATTGC
G-3′;
Comp15:
(SEQ ID NO: 19)
5′-GTAAAGCTTTTTTTTTTTTACAACCACCACCACC-3′;
F50-48:
(SEQ ID NO: 18)
5′-GGTGGTGGTGGTTGTGGTGGTGGTGGTCTAAAGTTTTGTCGTGAAT
TGCG-3′;
Comp15-48:
(SEQ ID NO: 19)
5′-GTAAAGCTTTTTTTTTTTTACAACCACCACCACC-3′;
F50-73
(SEQ ID NO: 20)
5′-GGTGGTGGTGGTTGTGGTGGTGGTGGTAGAGCTTGACGGGGAAATC
AAAA-3′;
Comp15-73:
(SEQ ID NO: 21)
5′-TGTAGCATTTTTTTTTTTTACAACCACCACCACC-3′;
F50-97:
(SEQ ID NO: 22)
5′-GGTGGTGGTGGTTGTGGTGGTGGTGGCGAGAAAGGAAGGGAACAAA
CTAT-3′;
Comp15-97:
(SEQ ID NO: 23)
5′-TGAGTTTCTTTTTTTTTTTACAACCACCACCACC-3′;
F50-120:
(SEQ ID NO: 24)
5′-GGTGGTGGTGGTTGTGGTGGTGGTGGATAGGAACCCATGTACAAAC
AGTT-3′;
Comp15-120:
(SEQ ID NO: 25)
5′-CAAGCCCATTTTTTTTTTTTACAACCACCACCACC-3′;
F50-144:
(SEQ ID NO: 26)
5′-GGTGGTGGTGGTTGTGGTGGTGGTGGCACCACCCTCATTTTCCTAT
TATT-3;
Comp15-144:
(SEQ ID NO: 27)
5′-CCGCCAGCTTTTTTTTTTTACAACCACCACCACC-3′;
F50-169:
(SEQ ID NO: 28)
5′-GGTGGTGGTGGTTGTGGTGGTGGTGGCTACATTTTGACGCTCACCT
GAAA-3′;
or
Comp15-169:
(SEQ ID NO: 29)
5′-CCCTCAGTTTTTTTTTTTTACAACCACCACCACC-3′.

In some embodiments, the capture strand is extended with ssNA comprising four binding sites to form a complex with thrombin-NA molecules.

In some embodiments, ssNA nanostructure further comprises one or more functional strand of NA operably linked to an aptamer for targeting delivery of the nanostructure forming a targeting strand.

In some embodiments, the aptamer is specific for nucleolin. In some embodiments, the aptamer that is specific for nucleolin is an F50 AS1411 aptamer having the sequence: 5′-GGTGGTGGTGGTTGTGGTGGTGGTGG-3′ (SEQ ID NO: 30). In some aspects, the targeting strand comprises a domain comprising a polynucleotide sequence for attaching to a therapeutic agent described herein. In some embodiments, when the nucleolin-specific aptamer is presented to nucleolin on a tumor cell surface, the aptamer will competitively bind to the surface-bound nucleolin. In some embodiments, when the RNA nanostructure scaffold is in the form of a tube comprising a fastener strand wherein the fastener strand is a nucleolin-specific aptamer, when the aptamer competitively binds to the tumor cell surface-bound nucleolin, the fastener strand will release from one or all of the RNA nanostructure scaffolds wherein the scaffold will change shape from a tube to an open rectangular sheet.

In some embodiments, one or more targeting strands are positioned at one or more corners of the rectangular sheet.

In some embodiments, one or more capture strands is operably linked to a fluorescent dye to form an imaging strand.

In some embodiments, the therapeutic agent is operably linked to the top surface of the rectangular sheet.

In some embodiments, the therapeutic agent is operably linked to the bottom surface of the rectangular sheet.

In some embodiments, the therapeutic agent is operably linked to an imaging agent. In some embodiments, the imaging agent is a fluorescent dye.

In some embodiments, the therapeutic agent is a protein.

In some embodiments, the therapeutic agent is thrombin.

In some embodiments, the therapeutic agent is siRNA, a chemotherapeutic agent or a peptide therapeutic agent.

In some embodiments, the thrombin is operably linked to the functional strand of ssNA by means of a sulfosuccinimidyl-4-(N-maleimidomethyl) cyclohexane-1-carboxylate (sulfo-SMCC) as a bifunctional crosslinker.

In some embodiments, the nanostructure comprises four thrombin molecules.

In some embodiments, the target molecule is nucleolin.

In some embodiments, the thrombin is operably linked to an imaging agent. In some embodiments, the imaging agent is a fluorescent dye.

Certain embodiments of the invention provide a pharmaceutical composition comprising the DNA nanostructure described herein.

In some embodiments, the composition further comprises at least one therapeutic agent.

In some embodiments, the at least one therapeutic agent is a chemotherapeutic drug (e.g., doxorubicin).

Certain embodiments of the invention provide a method of treating a disease or disorder in a subject, comprising administering to the subject a therapeutically effective amount of the NA nanostructure or pharmaceutical composition as described herein.

In some embodiments, the disease or disorder is cancer.

In some embodiments, the cancer is breast cancer, ovarian cancer, melanoma or lung cancer.

Certain embodiments of the invention provide a use of the ssNA nanostructure or a composition as described herein for the manufacture of a medicament for inducing a tumor necrosis response in a subject (e.g., a mammal, which can include or exclude a human).

Certain embodiments of the invention provide a ssNA nanostructure or a composition as described herein for the prophylactic or therapeutic treatment a disease or disorder.

Certain embodiments of the invention provide a kit comprising the ssNA nanostructure or a composition as described herein and instructions for administering the ssNA nanostructure/composition to a subject to induce an immune response or to treat a disease or disorder. In some embodiments, the kit further comprises at least one therapeutic agent.

The invention also provides processes disclosed herein that are useful for preparing a ssNA nanostructure described herein.

In some embodiments, one or more agents (e.g., 1, 2, 3, 4, 5, 6, 7, 8, 9, 10, or more) are operably linked to the ssNA nanostructure. The agents are selected from diagnostic agents or therapeutic agents. In some embodiments, at least one diagnostic agent is operably linked to the ssNA nanostructure. In some embodiments, at least one therapeutic agent is operably linked to the ssNA nanostructure. In some embodiments, at least one diagnostic agent and at least one therapeutic agent are operably linked to the ssNA nanostructure.

As used herein, the term “therapeutic agent” includes agents that provide a therapeutically desirable effect when administered to an animal. In some embodiments, the subject is a mammal, which can include or exclude a human. The agent is of natural or synthetic origin. In some embodiments, the therapeutic agent can include or exclude a nucleic acid, a polypeptide, a protein, a peptide, a radioisotope, saccharide or polysaccharide or an organic compound. In some embodiments, the organic compound can include or exclude a small molecule. The term “small molecule” includes organic molecules having a molecular weight of less than about, e.g., 1000 daltons. In one embodiment, a small molecule can have a molecular weight of less than about 800 daltons. In another embodiment, a small molecule can have a molecular weight of less than about 500 daltons.

In some embodiments, the therapeutic agent is an immuno-stimulatory agent, a radioisotope, a chemotherapeutic drug or an immuno-therapy agent. In some embodiments, the immune-stimulatory agent is selected from an antibody or an antibody fragment. In some embodiments, the antibody fragment is a nanobody, scFv, or camelid antibody. In some embodiments, the therapeutic agent is a vaccine, which can include or exclude a cancer vaccine. In some embodiments, the therapeutic agent is a tumor targeting agent, which can include or exclude a monoclonal tumor-specific antibody or an aptamer. In some embodiments, the therapeutic agent is an antibody. In some embodiments, the antibody is a monoclonal antibody. In some embodiments, the monoclonal antibody is an anti-PD1 antibody. In some embodiments, the therapeutic agent is an antigen. In some embodiments, the antigen is selected from a tumor associated antigen or a tumor specific antigen. In some embodiments, the therapeutic agent is a tumor antigen peptide(s). In some embodiments, the therapeutic agent is an RNAi molecule. In some embodiments, the RNAi molecule is selected from siRNA, shRNA, or miRNA. In some embodiments, the therapeutic agent is a small molecule drug. In some embodiments, the therapeutic agent is thrombin.

In some embodiments, the therapeutic agent is a chemotherapeutic agent. In some embodiments, the chemotherapeutic agent is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: cribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In some embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CecNU (Lomustine), CecNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimctinib), Cyramza (Ramucirumab), CytosarU (Cytarabinc), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginasc), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex (Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (Ibrutinib), Imfinzi (Durvalumab), Imlygic (Talimogene Laherparepvec), Inlyta (Axitinib), Intron A alfab (Interferon alfa-2a), Iressa (Gefitinib), Istodax (Romidepsin), Ixcmpra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mckinist (Tramctinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargasc), Oncovin (Vincristine), Opdivo (Nivolumab), Panretin (Alitretinoin), Paraplatin (Carboplatin), Perjeta (Pertuzumab), Platinol (Cisplatin), PlatinolAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), Vitrakvil (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus) ,Zydelig (Idelalisib), Zykadia (Ceritinib), and Zytiga (Abiraterone).

### Linkages

The linkage between the agent(s) and the ssNA nanostructure connects the ssNA nanostructure and the agent and does not interfere with the function of the agent or the ssNA nanostructure. In some embodiments, chemistries that link the agent to an oligonucleotide can include or exclude disulfide linkages, amino linkages, and covalent linkages. In some embodiments, the linker can include or exclude aliphatic or ethylene glycol linkers. In some embodiments the linker can include or exclude phosphodiester, phosphorothioate and/or other modified linkages. In some embodiments, the linker is a binding pair. As used herein, the term “binding pair” refers to two molecules which interact with each other through any of a variety of molecular forces which can include or exclude ionic, covalent, hydrophobic, van der Waals, and hydrogen bonding, so that the pair have the property of binding specifically to each other. Specific binding means that the binding pair members exhibit binding to each other under conditions where they do not bind to another molecule. Binding pairs can include or exclude biotin-avidin, hormone-receptor, receptor-ligand, enzyme-substrate probe, IgG-protein A, antigen-antibody, aptamer-target and the like. In some embodiments, a first member of the binding pair comprises avidin or streptavidin and a second member of the binding pair comprises biotin. In some embodiments, a first member of the binding pair comprises nickel and a second member of the binding pair comprises a His-tag. In some embodiments, the binding pair is another affinity ligand interaction.

### Therapeutic Agents to be Administered

In some embodiments, the therapeutic agent is thrombin. In some embodiments, the therapeutic agent is a tumor targeting agent. In some embodiments, the therapeutic agent is an RNAi molecule. In some embodiments, the RNAi molecule is selected from e.g. siRNA, shRNA, and miRNA.

### Certain Definitions

As used herein, the term “about” means±10%.

As used herein, the term “knot” refers to a continuous or near-continuous double-stranded nucleic acid sequence embedded in 3-dimensional Euclidian space comprising a crossing number larger than zero. A crossing number is a knot invariant that shows the smallest number of crossings in any diagram of the knot, representing the topological complexity of a knot. The knot can be closed or open. An open knot comprises nucleic acid strands which are not fully ligated. In some embodiments, an open knot is also referred to as a “pseudoknot.” An open knot comprises one or a plurality of 3′ terminae and one or a plurality of 5′ terminae. A closed knot comprises nucleic acid strands which are continuous. The nucleic acids can be selected from: DNA, RNA, or mixtures thereof. In some embodiments, the DNA comprises natural or unnatural DNA base-pairs. In some embodiments, the knots of this disclosure exclude compact structures wherein a first double-stranded nucleic acid strand is parallel to a second double-stranded nucleic acid strand because the compact structures will not facilitate the correct folding of DNA. In some embodiments, knots of this disclosure are constructed to exhibit wireframe networks which are better candidates for constructing knotted structures, because they offer more space for ssNA chains to thread through the structures. In some embodiments, the knot further comprises a plurality of hairpins with an extended quasi-continuous double-helical stem region.

As used herein, the term “knotted nanostructure” refers to a nanostructure comprising knots and a series of crossover events occurring in periodic order. In some embodiments, the periodic order is a single phase (e.g., ever 8 bp). In some embodiments, the periodic order comprises a plurality of phases (e.g., every 8 bp, and every 3 bp). In some embodiments, the periodic order comprises one, two, three, four, five, six, seven, eight, nine, or ten phases. In some embodiments, the phase is every 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or more basepairs (bp) in length. In some embodiments, the knotted nanostructure comprises a crossing number equal to zero, which is topologically equivalent to an unknotted circle.

In some embodiments, the knotted nanostructures are greater than 10 nm along any given axis of the nanostructure. In some embodiments, the knotted nanostructures are greater than 10, 20, 30, 40, 50, 60, 70, 80, 90, 100, 110, 120, 130, 140, 150, 160, 170, 180, 190, 200, 210, 22, 230, 240, 250, or higher nm along any given axis of the nanostructure.

As used herein, the term “operably-linked” refers to the association two chemical moieties so that the function of one is affected by the other, e.g., an arrangement of elements wherein the components so described are configured so as to perform their usual function.

As used herein, the term “nucleic acid” refers to deoxyribonucleotides or ribonucleotides and polymers thereof in either single-or double-stranded form, comprising monomers (nucleotides) containing a sugar, phosphate and a base that is either a purine or pyrimidine. Unless specifically limited, the term encompasses nucleic acids containing analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

As used herein, the terms “nucleotide sequence” and “nucleic acid sequence” refer to a sequence of bases (purines and/or pyrimidines) in a polymer of DNA or RNA, which can be single-stranded or double-stranded. In some embodiments, the nucleotide sequence comprises synthetic, non-natural or altered nucleotide bases, and/or backbone modifications (e.g., a modified oligomer, which can include or exclude a morpholino oligomer, phosphorodiamate morpholino oligomer or vivo-mopholino). The terms “oligo”, “oligonucleotide” and “oligomer” may be used interchangeably and refer to such sequences of purines and/or pyrimidines. The terms “modified oligos”, “modified oligonucleotides” or “modified oligomers” may be similarly used interchangeably, and refer to such sequences that contain synthetic, non-natural or altered bases and/or backbone modifications (e.g., chemical modifications to the internucleotide phosphate linkages and/or to the backbone sugar).

Modified nucleotides can include or exclude alkylated purines; alkylated pyrimidines; acylated purines; and acylated pyrimidines. These classes of pyrimidines and purines can include or exclude pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psucouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Backbone modifications can include or exclude chemical modifications to the phosphate linkage. The chemical modifications to the phosphate linkage can include or exclude e.g. phosphorodiamidate, phosphorothioate (PS), N3′phosphoramidate (NP), boranophosphate, 2′,5′phosphodiester, amide-linked, phosphonoacetate (PACE), morpholino, peptide nucleic acid (PNA), inverted linkages (5′-5′ and 3′-3′ linkages)) and sugar modifications (e.g., 2′-O-Me, UNA, LNA).

The oligonucleotides described herein may be synthesized using solid or solution phase synthesis methods. In some embodiments, the oligonucleotides are synthesized using solid-phase phosphoramidite chemistry (U.S. Pat. No. 6,773,885, herein incorporated by reference) with automated synthesizers, herein incorporated by reference. Chemical synthesis of nucleic acids allows for the production of various forms of the nucleic acids with modified linkages, chimeric compositions, and nonstandard bases or modifying groups attached in chosen places through the nucleic acid's entire length. In some embodiments, the oligonucleotides described herein may be synthesized using enzymatic methods which can include adding single-bases via an enzyme.

Some embodiments of the invention encompass isolated or substantially purified nucleic acid compositions. As used herein an “isolated” or “purified” DNA molecule or RNA molecule refers to a DNA molecule or RNA molecule that exists apart from its native environment and is therefore not a product of nature. An isolated DNA molecule or RNA molecule may exist in a purified form or may exist in a non-native environment. In some embodiments, the non-native environment can include or exclude a transgenic host cell. In some embodiments, the terms “isolated” or “purified” includes a nucleic acid molecule which is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “portion” or “fragment,” as it relates to a nucleic acid molecule, sequence or segment of the invention, when it is linked to other sequences for expression, is meant a sequence having at least 80 nucleotides, at least 150 nucleotides, or at least 400 nucleotides. If not employed for expressing, a “portion” or “fragment” means at least 9, at least 12, at least 15, or at least 20, consecutive nucleotides, e.g., probes and primers (oligonucleotides), corresponding to the nucleotide sequence of the nucleic acid molecules of the invention.

“Recombinant DNA molecule” is a combination of DNA sequences that are joined together using recombinant DNA technology and procedures to join together DNA sequences as described in Sambrook and Russell, Molecular Cloning: A Laboratory Manual, Cold Spring Harbor, NY: Cold Spring Harbor Laboratory Press (3^{rd} edition, 2001), herein incorporated by reference.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleotide sequences: (a) “reference sequence,” (b) “comparison window,” (c) “sequence identity” (d) “percentage of sequence identity,” (e) “substantial identity” and (f) “complementarity”.
(a) As used herein, “reference sequence” is a defined sequence used as a basis for sequence comparison. A reference sequence may be a subset or the entirety of a specified sequence. In some embodiments, the specified sequence can include or exclude a segment of a full-length cDNA or gene sequence, or the complete cDNA or gene sequence.
(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally is 30 contiguous nucleotides, 40 contiguous nucleotides, 50 contiguous nucleotides, 100 contiguous nucleotides, or longer. Those of skill in the art understand that to avoid a high similarity to a reference sequence due to inclusion of gaps in the polynucleotide sequence a gap penalty is typically introduced and is subtracted from the number of matches.

In some embodiments, methods of alignment of sequences for comparison are determined by mathematical algorithm. In some embodiments, the determination of percent identity, including sequence complementarity, between any two sequences is accomplished using a mathematical algorithm. In some embodiments, such mathematical algorithms can include or exclude the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993), all of which are herein incorporated by reference.

In some embodiments, computer implementations of these mathematical algorithms are utilized for comparison of sequences to determine sequence identity or complementarity. Such implementations can include or exclude: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. A test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

Comparison of nucleotide sequences for determination of percent sequence identity may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.
(c) As used herein, the terms “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. In some embodiments, the identity between any two nucletic acid sequences is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, 95%, 96%, 97%, 98%, or 99%.
(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described herein using standard parameters.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. As used herein, the term “bind(s) substantially” refers to complementary hybridization between a probe nucleic acid and a target nucleic acid and embraces minor mismatches that in some embodiments is accommodated by reducing the stringency of the hybridization media to achieve the desired detection of the target nucleic acid sequence.
(f) The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Two nucleic acids are substantially complementary to each other when at least about 50%, at least about 60%, or at least about 80% of corresponding positions in each of the molecules are occupied by nucleotides which normally base pair with each other (e.g., A:T (A:U for RNA) and G:C nucleotide pairs).

As used herein, the term “derived” or “directed to” with respect to a nucleotide molecule means that the molecule has complementary sequence identity to a particular molecule of interest.

The term “subject” as used herein refers to humans, higher non-human primates, rodents, domestic, cows, horses, pigs, sheep, dogs and cats. In one embodiment, the subject is a human.

The term “therapeutically effective amount,” in reference to treating a disease state/condition, refers to an amount of a therapeutic agent that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The invention will now be illustrated by the following non-limiting Example.

### Example 1

Synthetic topological DNA nanostructures have previously been constructed by creating topological nodes based on B-form/Z-form double-stranded DNA (dsDNA) helices (J. E. Mueller, et al. J Am Chem Soc 113, 6306-6308 (1991); S. M. Du, et al. J Am Chem Soc 114, 9652-9655 (1992); S. M. Du, et al. J Am Chem Soc 117, 1194-1200 (1995); C. D. Mao, et al. Nature 386, 137-138 (1997)), paranemic crossovers (Y. P. Ohayon et al., ACS Nano 9, 10296-10303 (2015); Y. R. Ohayon et al., ACS Nano 9, 10304-10312 (2015)), and DNA four-way junctions (D. Liu, et al. Nat Chem 8, 907-914 (2016)). In contrast to the strategies that rely on the design of individual topological nodes and finding the appropriate DNA motifs/interactions with which to assemble such nodes, a completely different approach is to fold one long single-stranded DNA (ssDNA) chain into programmable topologies with periodic crossover events.

This disclosure includes a method for creating topological knots comprising single-stranded DNA (ssDNA) and/or single-stranded RNA (ssRNA) with high crossing numbers at periodic intervals. As used herein, the term “crossing” refers to a knot invariant that shows the smallest number of crossings in any diagram of the knot, representing the topological complexity of a knot (J. W. Alexander, T Am Math Soc 30, 275-306 (1928)). Acrossing applied to ssDNA or ssRNA knots is the cross-hybridization of two separate strands resulting in a pair of double-helix structures. The number of “crossings” in a NA nanostructure is the “crossing number,” Each of the ssDNA or ssRNA knots are folded and then self-assembled from a replicable single-stranded nucleic acid molecule with sizes ranging from 1800 to 7500 nucleotides (nt). In some embodiments, this disclosure includes the design, construction, and characterization of various ssDNA knotted nanostructures that had different crossing numbers and that displayed two-dimensional (2D) patterns. The folding yield of the knotted nanostructures was optimized by programming a step-wise hierarchical folding pathway through sequence design in the paranemic cohesion regions. In some embodiments, the method further comprises a series of design rules to significantly improve the yield of well-formed target nanostructures. In some embodiments, the target nanostructures can include or exclude ssRNA knotted nanostructures and three-dimensional (3D) ssDNA knotted nanostructures. In some embodiments, the 3D knotted nanostructures were characterized by cryogenic transmission electronic microscopy (cryo-EM) single particle reconstruction, which confirmed that the nanostructures had assumed the designated geometry.

Enzymatically transcribed and replicatable knotted nanostructures, comprising single-stranded nucleic acids that can self-fold into molecular knots of customized shapes, enables significant reduction in the cost of production of a multi-stranded DNA nanostructure system created from synthetic DNA. In some embodiments, the single-stranded topology enables for the selection and generation of knotted nanostructures with designed functions by using in vitro evolution. This disclosure further provides for a fundamental and general platform for constructing nucleic acids nanostructures with unprecedented complex molecular topologies.

DNA parallel crossover motifs were used as the modular building blocks with a node-edge network as the geometric blueprint to create ssDNA knotted nanostructures. Unlike DNA antiparallel double crossover motifs that contained localized ssDNA, all of the strands of a parallel crossover motif have both 5′ and 3′ ends that are extended to the terminals, which in some embodiments can be readily connected with other motifs into one long single strand. The use of a parallel crossover motif as the structural building block provides a basic geometric foundation that enables single-stranded routing.

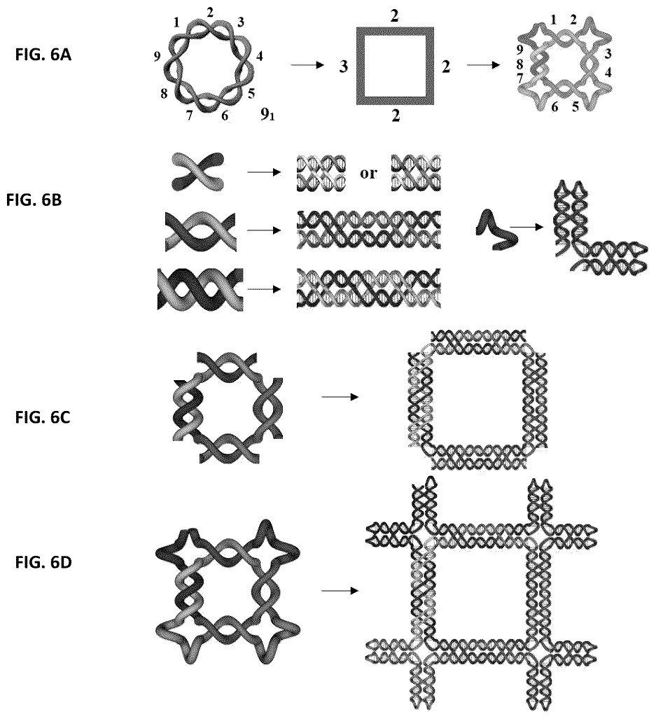

In comparison with the compact parallel or antiparallel helical arrangements, wireframe networks are better candidates for constructing knotted nanostructures, as they offer more space for DNA chains to thread through during the early formation of partial structures. In some embodiments, knot 9₁ (Alexander-Briggs notation) is assembled by either connecting 9 right-handed X-shaped junction tiles together, as shown in FIG. 1A or by threading a single chain through itself 9 times, as shown in FIG. 1B. Two parallel crossovers were separated by 4 or 6 base pairs to form an X-shaped topology, which represented one cross node in a knot, as shown in FIG. 1C. The 9 X-shaped DNA tiles were configured to be in the pattern of a square with 2, 2, 2, and 3 crossings on the four edges, respectively, as shown in FIG. 1D, and separated the adjacent junctions by 1 turn (10 or 11 bp) or 2 turns (21 bp) of the double stranded DNA. After connecting the nearest DNA strands and adding small linking structures at the four vertexes, the resulting design consisted of only one long ssDNA, as shown in FIGS. 6A-6D and FIG. 1D. The overall routing of the ssDNA was configured as a two-step process. First, half of the DNA chain folded back to partially pair with the other half of the DNA, leaving several unpaired single-stranded regions (˜4-6 nts) in between the perfectly paired regions (˜10, 11 or 21 bps). Then, the unpaired regions matched to each other by paranemic cohesive interactions and finally knotted into the target topology, as shown in FIG. 1D.

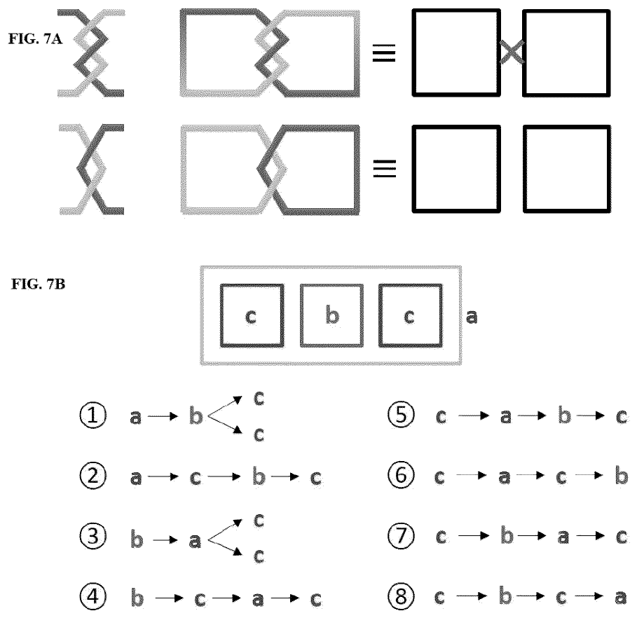

In some embodiments, the method comprises designing a topologically and kinetically favorable folding pathway for the successful formation of complex structures with high crossing numbers, optionally with periodic crossings. The inventors designed a hierarchical folding strategy to guide the knotting process in a prescribed order. In some embodiments, a knot with 23 crossings was assigned to a location on a three-column grid that is represented by a rectangle with three square cavities, as shown in FIG. 2C. To maintain the structural stability and rigidity of each edge, the the length and number of crossings on each edge was limited: in any six turns (63 bp) of a double helical DNA, only two or three crossings were allowed. In total, 23 crossings were assigned on the 10 edges, in which seven edges had two crossings and three edges had three crossings, as shown in FIG. 2C. In some embodiments, the folding pathway order was varied as shown in FIGS. 7A-7B. All of the possible combinations for the formation order of the crossings in the knot are listed and compared for their routing pathways, as shown in FIGS. 7A-7B.

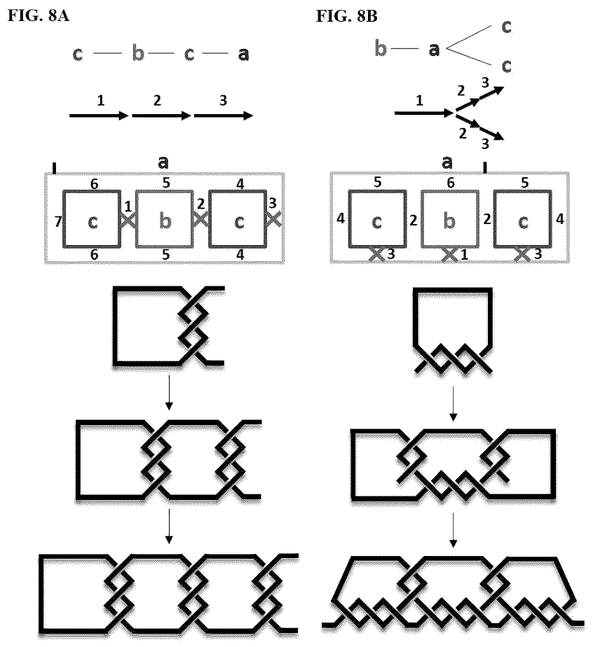

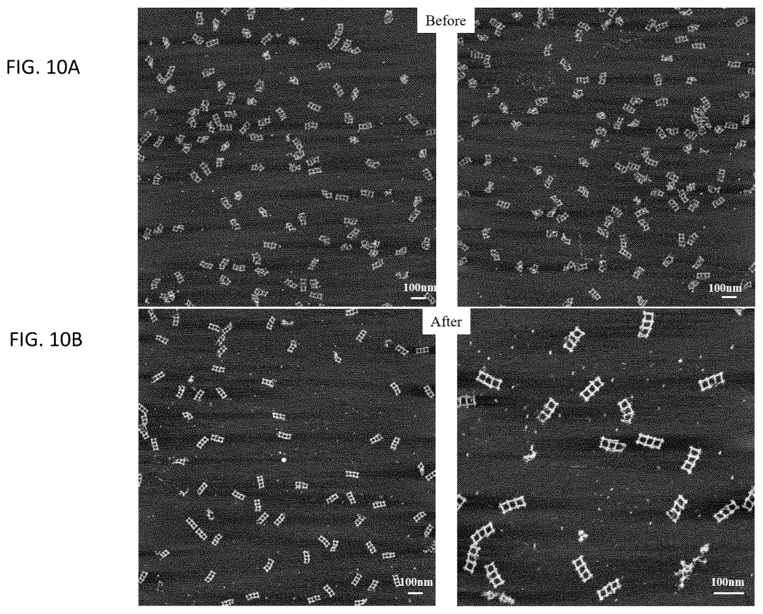

In some embodiments, the method comprises three design rules for optimizing the folding pathway: First, a linear folding path is better than a branched one, because the linear folding pathways involve two free ends that thread to form the loops in a sequentially ordered pathway, while the branched folding pathways have parallel steps that each involves a single free end to thread through the preformed loops. Based on an entropic point of view, the formation of two free ends looping with each other is expected to be easier than one free end threading itself through preformed loops, as shown in FIGS. 8A-8B. Second, a linear folding pathway has two possible folding directions, forwards or backwards. The best folding direction is determined in the early stage of folding when the unfolded portion of the strand is still long, and the strand avoids threading itself through any preformed structures, as shown in FIGS. 8A-8B. Third, it is expected that the edges with three crossings should fold before the edges with two crossings, since the cohesion force provided by three paranemic interactions (totally 12-18 bp) is expected to be stronger than that from two paranemic interactions (totally 8-12 bp). From the crossing positions and the grid layouts, the route for the chain threading is determined by specifying the order in which the chain visits each vertex and knots on each edge. FIG. 3A and FIGS. 9A-9D show the selected folding pathway for the three column grid knots. The folding yield of the two types of scaffold routings were compared by AFM imaging as shown in FIGS. 10A-10B. The selected linear folding pathway produced 57.9% (N=214) well-formed knotted NA nanostructures, while the branched pathway showed a yield as low as 0.9% (N=221).

The next step in the design procedure was to assign an appropriate sequence to enable the long ssDNA to create the structural and topological complexity. The inventors surprisingly discovered several criteria for generating a valid raw sequence: First, the ideal percentage of GC content in all regions of the DNA sequences was determined to be between 30% and 70%, since any GC content outside of this range would adversely affect the DNA synthesis. Second, depending on the size of the ssDNA, every segment that was 6-8 bases long was treated as one unit, to evaluate the uniqueness of the DNA sequence. The specificity of recognition between the designed base pairings rely on the uniqueness of the DNA sequences. Third, the repeating length of G was limited to 4 nt. A raw sequence was obtained by using the inherent algorithm of the Tiamat software, and in adherence with these rules. Then, the raw sequence was inspected manually and several modifications were made: The local sequences that form the paranemic crossovers were checked to make sure that each of the crossovers were stable; the GC content in each of the paranemic cohesion regions were designed individually and inter-dependently as they needed to be compared with one another. It was necessary that all of the paranemic cohesions would have a sequentially decreasing melting temperature, ordered according to the predetermined folding pathway. Lastly, the uniqueness of the paranemic cohesions was optimized independently, such that mismatches and cross-talking in the second step of the folding were minimized.

Both the chemical and enzymatic synthesis of long ssDNA molecules are technically challenging, because the chain possesses a large portion of self-complementarity. As shown in the folding pathway, the ssDNA molecule will first form a long hairpin structure with the 5′ and 3′ ends meeting each other. The full-length ssDNA strand was divided into two equal halves, with each strand lacking significant secondary structures. Each half was then inserted into plasmids as double stranded genes, and then amplified them by cloning. The two dsDNA genes were obtained separately from the plasmids by restriction enzymes digestion (EcoRI+XbaI and XbaI+HindIII respectively) and were then ligated together with a linearized phagemid vector, pGEM-7zf(−), as depicted in FIG. 11A. To obtain the full-length ssDNA molecule, the recombinant M13 phage was replicated in E. coli with the assistance of a helper plasmid, pSB4423 (A. N. Marchi, et al., Nano Lett 14, 5740-5747 (2014), herein incorporated by reference). Because the helper plasmid, pSB4423, does not contain a phage replication origin, only the phagemid vector containing the full-length ssDNA gene was able to act as a template for the phage DNA replication. After the extraction and purification of the recombinant phage DNA, EcoRV digestion was performed to cut out the target ssDNA, as shown in FIG. 11A. Native agarose gel electrophoresis was used to separate the target ssDNA from the phagemid vector ssDNA, as shown in FIG. 11B. The long ssDNA strands were synthesized and amplified at a nanomole quantity (with 1 L scale of E. coli culture) and high purity using the aforementioned method. The ssDNA strand was obtained, then self-assembled (folded) in a 1×TAE-Mg buffer with a 12 hour or 24 hour annealing ramp from 65° C. to 25° C.). The folded products were then characterized by using AFM imaging, gel electrophoresis and/or cryo-EM imaging.

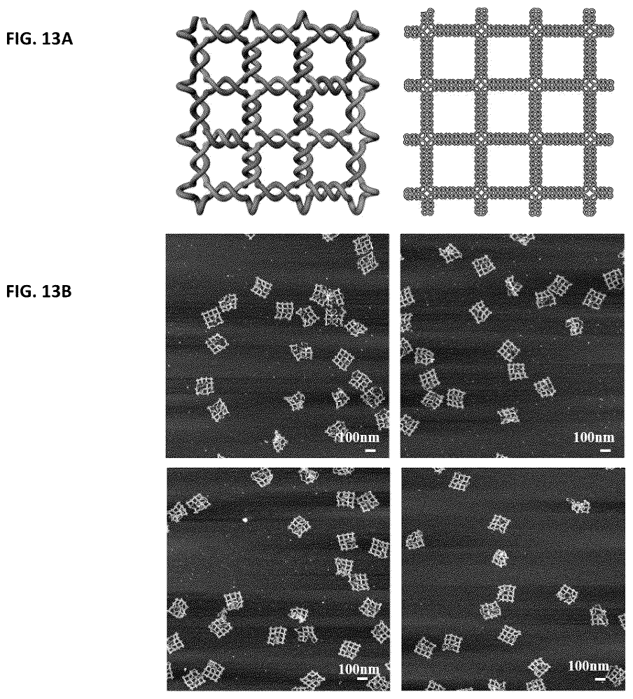

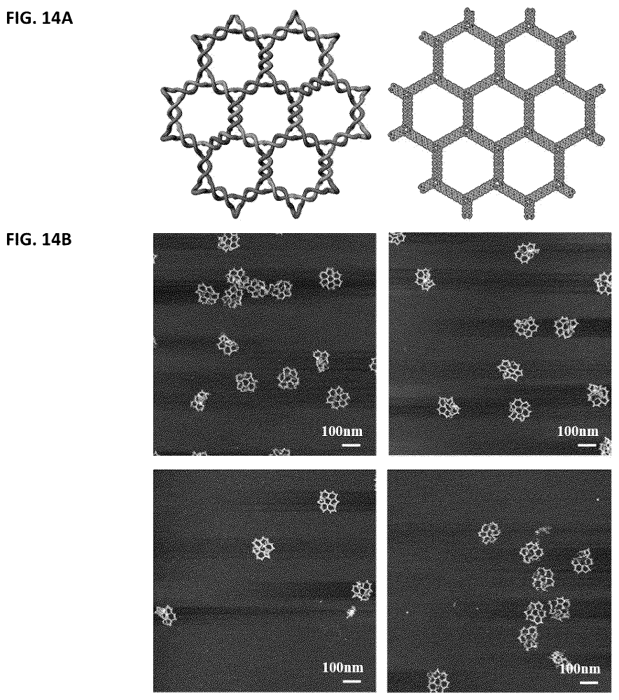

In some embodiments, this disclosure provides for methods of creating complex DNA knotted nanostructures with increasing crossing numbers. A 3 by 3 square grid of DNA knots with 57 crossed nodes was designed with an optimized linear folding pathway, as shown in FIG. 2D and FIG. 13B. Other geometric layouts were also used following similar design principles. A large molecular knot with 67 crossings in a hexagonal lattice was also designed and constructed. High resolution AFM images confirmed the successful formation of the target nanostructures, as shown in FIG. 2E and FIG. 14A-14B.

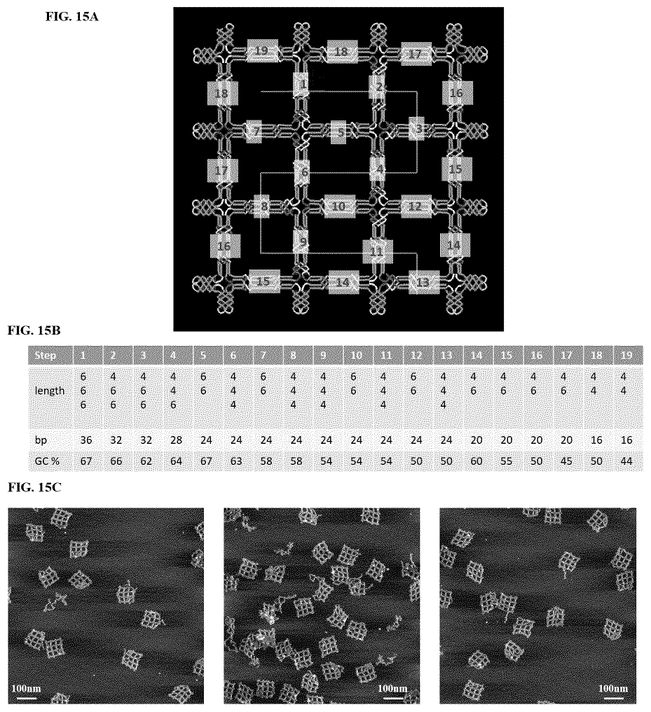

Smaller knot nanostructures with crossing numbers 9 and 23 folded well with yields as high as 69% (N=103) and 58% (N=214), respectively, as shown in FIGS. 2A& C, 12A-12B and 10A-10B. However, as the crossing number of the knot increased to 57 or 67, the folding yield dropped significantly and in the images, only 1.2% of the resulting nanostructures were perfectly formed with the 57 crossing number (N=327) and none of the resulting nanostructures were perfectly formed with the 67 crossing numbers (N=58) based on single molecule analysis by high-resolution AFM imaging, as shown in FIGS. 2D&E, 13A-13B and 14A-14B. Most of the formed nanostructures examined exhibited some degree of various folding defects, as shown in FIGS. 2D-2E. With such a high complexity, even the hierarchical folding optimization did not significantly increase the overall yield, as shown in FIGS. 15A-15C. The folding behaviors in the ssDNA knotted nanostructures were remarkably different than that of the classic DNA nanostructures. To make the target knotted nanostructure, the ssDNA chain needed to fold following an exactly defined order. If one crossing was misfolded in an earlier stage, it would be impossible (or at least extremely difficult) for it to correct itself afterwards. The yields of those knotted nanostructures were not surprisingly low when compared to the yields of the chemical synthesis reactions that contained multiple steps. The average yield for each knotting step (crossing step) was at least 90%, and in the low crossing number cases, the single step yield was as high as ˜96%.

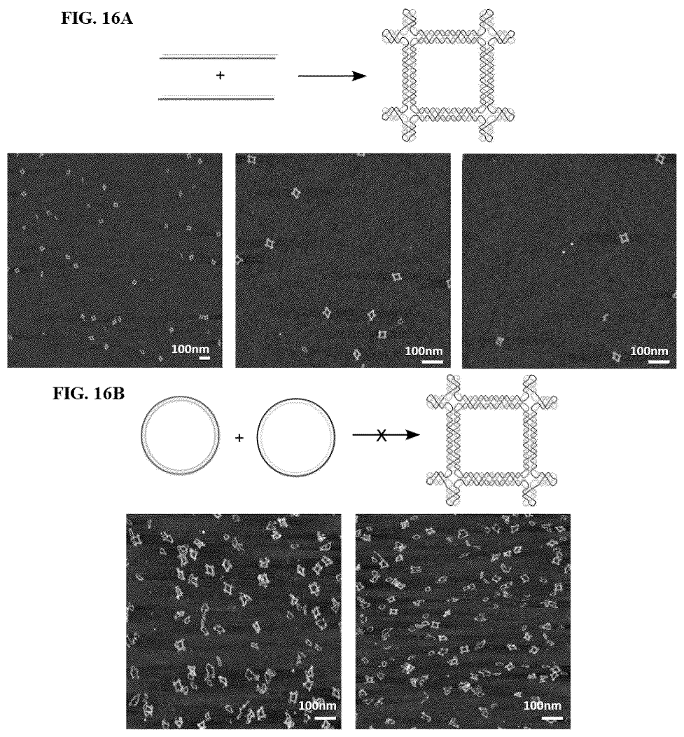

As most of the high crossing number (57 and 67) ssDNA knotted nanostructures were characterized by high resolution AFM imaging, as shown in FIGS. 2A-2E, one question was raised: whether or not the formation of the defected nodes in the final knotted nanostructure could be truly and completely identified with AFM imaging. A link structure was designed with 8 nodes as a topological control, as shown in FIGS. 4A-4B. The aforementioned link structure contained 2 dsDNA rings (each only partially complementary), which connected to each other through 8 paranemic cohesions. The aforementioned structure was formed by annealing two linear dsDNAs (each pre-formed from two ssDNAs) with 8stretches of mismatches (bubbles) 6 nt each, as shown in FIGS. 4A-4B and FIGS. 16A-16B. The mismatches within these two linear dsDNAs would interact with their counterparts to form the stable paranemic cohesions. 9-bp sticky ends that extended from both ends of the dsDNAs closed the two rings after the formation of the correct structure. This link structure assembled well, as characterized by high resolution AFM imaging, as shown in FIG. 4A. On the contrary, if the two linear dsDNAs were first ligated to form closed ring structures, these two dsDNA rings would not be able to assemble into the fully inter-locked loop nanostructure. As expected, although the two dsDNA rings could still bind with each other partially through some of the paranemic cohesion interactions, as shown in FIGS. 4B and 16A-16B, extensive defects were observed in all of the nanostructures, as shown in FIGS. 3A-3D and 16A-16B, which resembled those observed in FIGS. 2C and D with high crossing numbers. This experiment confirmed that the defects observed for the NA knotted nanostructures with high crossing numbers under high resolution AFM imaging were real defects that were due to mis-formed crossings, but not due to the scratching or damaging of the nanostructures by the AFM tip.

In some embodiments, this disclosure provides for methods of creating ssRNA knotted nanostructures. An X-shaped RNA modular building block was designed, which was similar to the structures of DNA. Then, based on the 3D modeling of an A-form dsRNA helix (11 bp per helical turn, 19 degree inclination of base pairs) and the best geometric fitting, 8 (instead of 4 or 6) base pairs were chosen for the length of a paranemic crossover, as shown in FIGS. 17A-17C. For an 8 base-pair paranemic cohesion, a total of 4⁸=65536 possible sequences provided an adequate sequence space for the selection of unique complementarity to sufficiently avoid undesired interactions between the paranemic cohesion motifs. Second, given the 11 base pairs per turn of an A-form dsRNA, the lengths of the inter-motif stems were designed as alternating between 8 and 9 bp, as shown in FIGS. 17A-17C to achieve a structural repeating unit of 33 bps for three full helical turns (i.e. 8 bp+8 bp stem+8 bp+9 bp stem=33 bp=3 full turns). In this design the neighboring structural units were in line with each other without accumulating helical twist, and the final assembled nanostructure was expected to stay in 2D. Like the ssDNA 9₁ knot, 2, 2, 2, and 3 crossings on the four edges of a square were designed and the vertexes were looped to form one single-stranded RNA, as shown in FIG. 2B. After generating the appropriate sequences by following the same sequence design rules as the ssDNA knotted nanostructures, the dsDNA gene coding for the long RNA strand was first synthesized, and then the ssRNA molecule was obtained by an in vitro transcription reaction. After annealing, the AFM images revealed the successful formation of the ssRNA 9₁ knots, as shown in FIG. 2B and FIGS. 17A-17C.

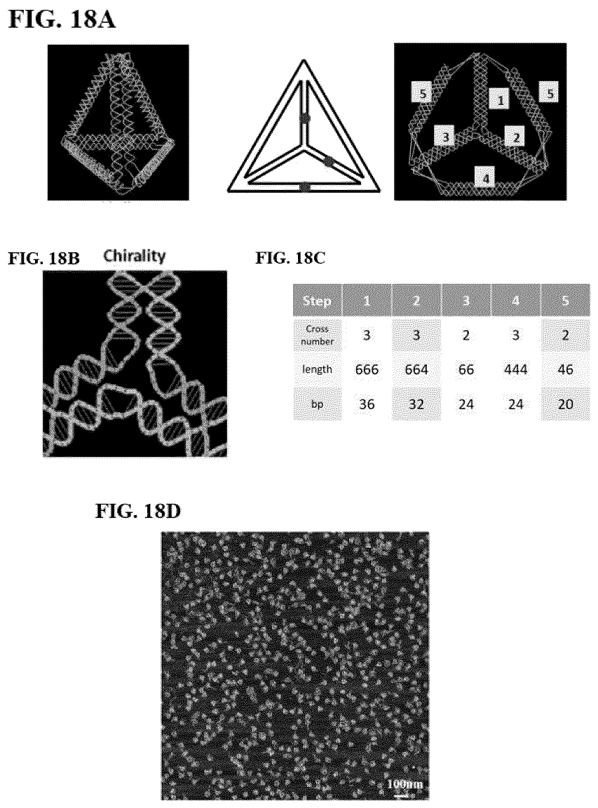

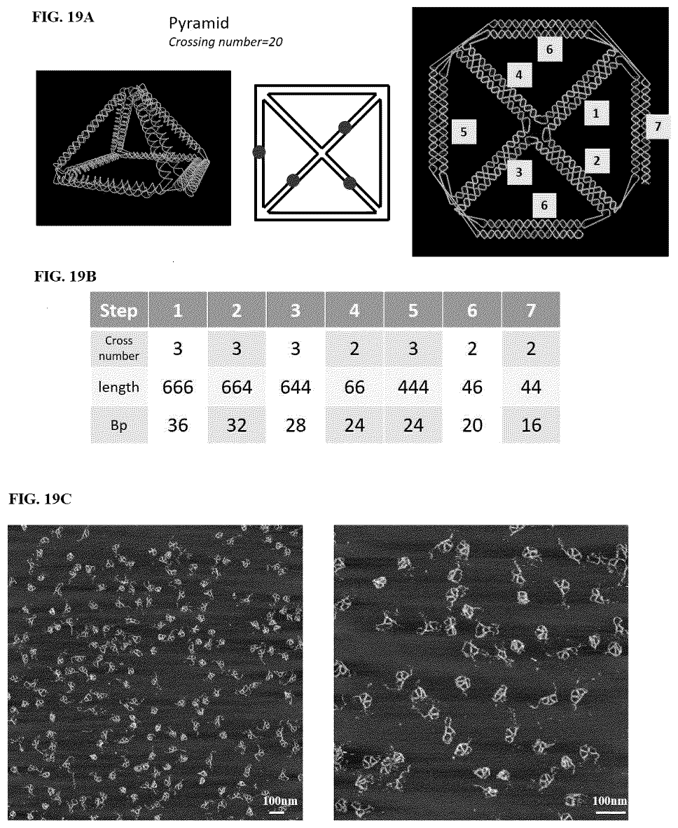

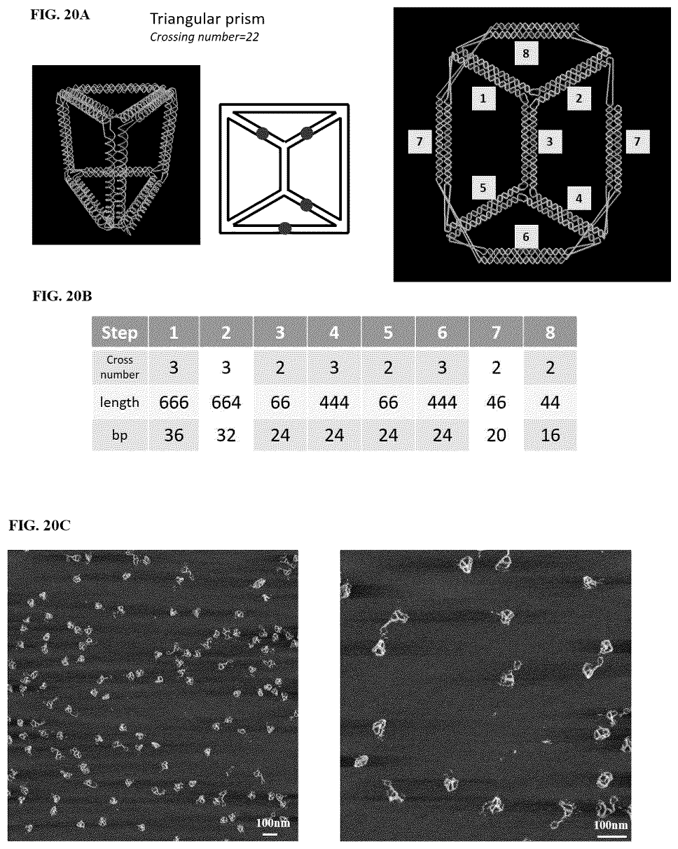

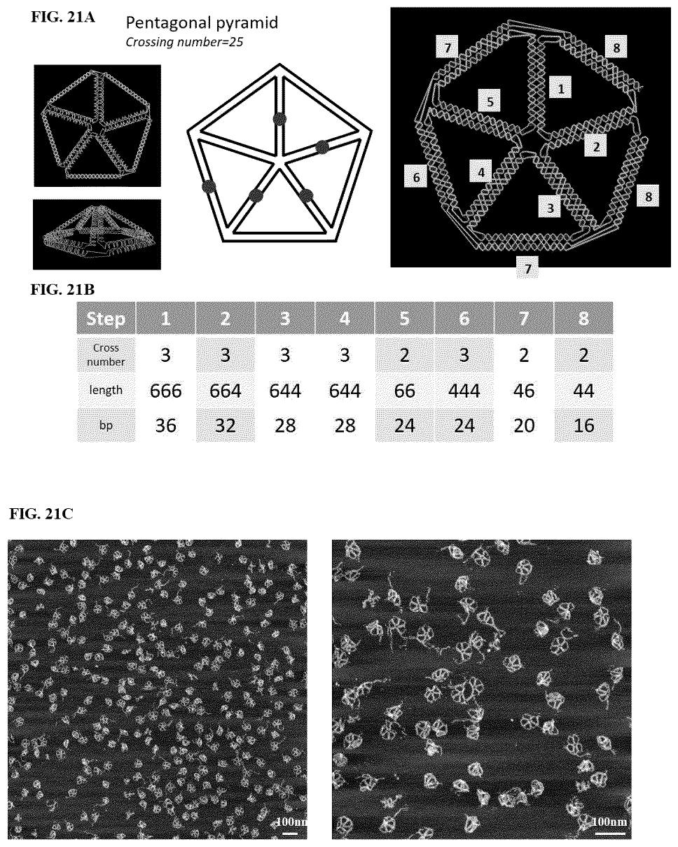

In some embodiments, this disclosure provides for methods of creating 4 ssDNA polyhedral meshes as knotted NA nanostructures: a tetrahedron, a pyramid, a triangular prism, and a pentagonal pyramid with crossing numbers 15, 20, 22, and 25, respectively, as shown in FIGS. 5A-5D. A schlegel diagram was used to transfer the 3D objects to their topologically equivalent 2D nets. Optimized folding pathways were designed carefully for step-wise hierarchical assembly and the corresponding ssDNA strands were designed, synthesized, and assembled. AFM images showed an abundance of well-folded 3D nanoparticles with the expected sizes, as shown in FIGS. 18A-18D, 19A-19C, 20A-20B, and 21A-21C. Single particle cryo-EM 3D reconstruction revealed that the overall conformations matched the designed geometries well, as shown in FIGS. 5A-5D and FIGS. 22A-22D and 23A-23C. The vertex design for the ssDNA knotted nanostructures was different from the multi-arm junction design based on the double crossover (DX) motif. There is a 5 bp difference in length between the two parallel dsDNAs that form each edge, leading to chiral vertices and inclined edges in the ssDNA knotted nanostructures, as shown in FIGS. 5A-5D. In some embodiments, the foregoing unique geometric feature can identify conformational diastereomers, which is when the structure is turned inside out, while satisfying all programmed Watson-Crick base pairing with the same network connectivity. The 3D reconstruction data indicated that the ssDNA knots preferred to point the major grooves inwards at the vertices.

In some embodiments, this disclosure provides for a method of folding single-stranded nucleic acids with completely custom-designed sequences to create ssDNA/ssRNA knotted nanostructures with highly complex topologies that are programmable, potentially replicable, and scalable. In some embodiments, the ssDNA knots described herein comprise one single routing strand that has high crossing numbers without the help of any auxiliary DNA. Various 2D and 3D shapes have been designed and successfully constructed with a surprisingly high yield (˜96%) of crossing steps. The same strategy has also been adapted for the design and construction of complex ssRNA knotted nanostructures. The ssDNA knotted nanostructures described herein comprise large DNA sequences (up to 7.5k bases) and highly complicated topology (as high as 67 crossing number).

In some embodiments, the method comprises the single-stranded folding process of nucleic acids. In some embodiments, the folding process is replicated and amplified in biological systems for cost-efficient, large scale production. In some embodiments, the single-stranded folding process is programmed in nucleic acid synthesis in target cells to produce nanostructures that harness functions in vivo.

In some embodiments, this disclosure provides for methods of using programmable ssDNA and ssRNA knots that enable the construction of engineered molecular devices and multivalent aptamers to use in directed evolution methods.

### Materials and Methods

### DNA/RNA Sequence Design

DNA and/or RNA nanostructures and sequences were designed using the Tiamat software (Yanlab.asu.edu/Tiamat.cxc) (S. Williams et al., Tiamat: A Three-Dimensional Editing Tool for Complex DNA Structures. 5347, 90-101 (2009)).

DNA and/or RNA sequences were generated by using the following criteria in the Tiamat software: (1) Unique sequence limit: 8 nt; (2) Repetition limit: 6-8 nt; (3) G repetition limit: 4 nt; (4) GC content: 0.45-0.55. Once sequences were generated, a few nucleotides were adjusted to eliminate the restriction enzyme targeting sequences (e.g. by EcoRI, EcoRV, HindIII and XbaI) for cloning purposes. Then, the raw sequences of the paranemic cohesion regions were inspected manually and several modifications were made: the local sequences that form the paranemic crossovers were checked to make sure that each of the crossovers were stable; the GC content in each paranemic cohesion region were designed individually and inter-dependently as they needed to be compared with one another so that all of the paranemic cohesions would have a strength that was ordered sequentially according to a predetermined folding pathway; lastly, the uniqueness of the paranemic cohesions was optimized independently, so that the occurrence of mismatches and cross-talking in the second step of the folding process were minimized. For ssRNA sequences, a T7 promoter sequence was followed by two or three consecutive Gs that were manually incorporated onto the 5′ end of the strand to facilitate efficient in vitro transcription reactions during gene synthesis.

### ssDNA/RNA Synthesis

The ssDNA sequences described herein were divided into two fragments with restriction sites added onto both ends. The first fragment contained EcoRI and XbaI restriction sites and the second one contained XbaI and HindIII restriction sites. The EcoRV sites were also manually added to both ends of the full-length sequence to facilitate the production of the final ssDNA. The two DNA fragments that were ordered as double stranded genes in plasmids were from Biobasic Company (Biobasic.com) with sequences that were verified through Sanger sequencing. The two DNA fragments that were cleaved from the plasmids by the restriction enzymes were then subcloned into an EcoRI and a HindIII linearized pGEM-7zf(−) vector (Promega). After sequencing verification, the pGEM-7zf(−) vector that contained the full ssDNA genes was co-transformed into E. coli DH5α competent cells along with a helper plasmid pSB4423, a kind gift from Dr. Stanley Brown (Niels Bohr Institute, Denmark). E. coli colonies were formed after overnight incubation at 37° C., and a single colony was inoculated into the 2×YT medium that had been supplemented with 2 mM MgSO₄ (Sigma-Aldrich) and grown at 37°° C. overnight with shaking at 250 rpm. During the overnight growth, the recombinant M13 phages were continuously produced and secreted into the medium. During the next day, the culture was centrifuged at 5,000 g for 15 min to pellet down the E. coli cells. The recombinant M13 phage was precipitated from the recovered supernatant with the addition of NaCl (to 30 g per liter) and PEG8000 (to 40 g per liter), and incubated in the 4° C. cold room for 1 hour. The precipitated phage was then collected by centrifugation at 4,500 g and 4° C. for 15 min. The recombinant phage DNA was isolated from the harvested phage. The phage ssDNA was digested by EcoRV enzyme (New England Biolabs) and resolved on a 1% agarose gel. The correct bands were sliced and purified by using a Monarch DNA Gel Extraction Kit (New England Biolabs).

For the ssRNA molecule synthesis, the DNA sequence with a T7 promoter at the 5′ end was first cloned into a pUC19 vector by using the same method as the ssDNA gene cloning process that was described above. The plasmid containing the ssRNA gene was linearized by using a HindIII enzyme (New England Biolabs) and the plasmid was purified by using a Phenol/chloroform extraction and ethanol precipitation. The in vitro transcription reaction was carried out by using the T7 RiboMAX Express Large Scale RNA Production System (Promega), following the manufacturer's instructions. The RNA molecules were then purified via a RNA Clean & Concentrator-25 kit (Zymo Research).

### ssDNA/RNA Nanostructure Assembly

The purified DNA and/or RNA molecules were diluted to 5-10 nM in 1×TAE-Mg buffer (40 mM Tris, 20 mM Acetic Acid, 2 mM EDTA and 12.5 mM Magnesium acetate, pH 8.0). The resulting solution was annealed from 65° C. to 25° C. with a cooling ramp of 1° C. per 20 minutes to form the NA nanostructures.

AFM characterization

All samples were imaged in “ScanAsyst mode in fluid,” using a Dimension FastScan microscope with PEAKFORCE-HiRs-F-A tips (Bruker Corporation). After annealing, 2 μl of each sample was deposited onto a freshly cleaved mica surface (Ted Pella, Inc.), and left to adsorb for 1 minute. Then, 80 μl of 1×TAE-Mg buffer and 2 μl 100 mM of a NiCl₂ solution was added onto the mica, and 40 μl of the same buffer was deposited onto the microscope tip. The samples were then scanned by following the manufacturer's instructions.

### Topological Control Experiments

Four single-stranded DNAs with customized sequences were synthesized by The Biobasic company (Biobasic.com) and then cloned into a pBluescript SK (+) vector (Biobasic), with the gene sequences flanked by two BtsCI restriction sites. The final plasmids were then co-transformed with pSB4423 to produce recombinant M13 phages. The phage particles and phage DNAs were then purified by using the same methods as described in the ssDNA synthesis section. The ssDNAs were cleaved off from the recombinant phage DNAs by using a BtsCI restriction enzyme (New England Biolabs) and were gel purified by using a 4% urea denaturing PAGE gel. The four ssDNAs were annealed into two sets of dsDNAs (partially hybridized) in the 1×annealing buffer (50 mM Tris-HCl pH8.0 and 100 mM NaCl). The two linear dsDNAs were mixed in a 20 nM concentration in a 1×TAE-Mg buffer, annealed from 65°° C. to 25° C. at 1° C. per 20 minutes to form the paranemic cohesion interactions, and were then characterized by AFM imaging. The sticky ends on the two sets of the dsDNAs were able to close the ring structure without ligation. In the second control experiment, the two linear dsDNAs were ligated separately with T4 DNA ligase in a 1×ligation buffer (Thermo Fisher Scientific) at room temperature for 1 hour to enablel them to form the two circular dsDNAs. The ligation products were treated with exonuclease I and exonuclease III (New England Biolabs) to remove any linear DNA. The solution was further purified using a Monarch PCR & DNA Cleanup Kit (New England Biolabs). The two circular dsDNAs were then mixed at 20 nM in a 1×TAE-Mg buffer, annealed from 65° C. to 25° C. at 1° C. per 20 minutes and characterized by AFM imaging.

### CryoEM Specimen Preparation and Data Acquisition

In the cryo-EM portion of the experiment, 2 μL of the aforementioned single stranded DNA nanostructure samples (concentrated to ˜0.3 μM using Amicon 100 kDa centrifugal filters) were applied onto a 200 mesh R1.2/1.3 holey carbon Quantifoil grid (Quantifoil Micro Tools GmbH) that was cleaned with acetone (Sigma-Aldrich) for 12 hours and glow discharged for 40 seconds before use. The grid was blotted for 3.5 seconds and immediately frozen in liquid ethane by using a Vitrobot Mark IV (FEI) with a constant temperature of 6° C. and with humidity levels at 100%. The grid was stored in liquid nitrogen until the imaging session. All of the grids were examined on a JEM2200FS (field emission gun) cryo-electron microscope (JEOL) that was operated under the following parameters: 200 kV, spot size 2, condenser aperture 70 μm, objective aperture 60 μm. The images were recorded under a low-dose condition on a direct detection device (DDD) (DE-20 4k×5k camera, Direct Electron, LP) while operating in movie mode at a recording rate of 24 raw frames per second. Other conditions included a 30,000× microscope magnification (corresponding to a calibrated sampling of 1.59 Å/pixel) and a dose of 40 electrons/Å² with a defocus ranging from 1.5 to 3 μm.

For the ssDNA tetrahedron sample, a total of 47 images were recorded on the DE-20 detector. Motion correction was performed by running the averages of 3 consecutive frames with the use of the DE_process_frames.py script (Direct Electron, LP). A total of 533 particle images were manually boxed, contrast transfer function corrected, and extracted with the use of EMAN2 (Tang, G., Peng, L., Baldwin, P. R., Mann, D. S., Jiang, W., Rees, I., and Ludtke, S. J. (2007) EMAN2: an extensible image processing suite for electron microscopy, Journal of structural biology 157, 38-46.) Approximately 50 particles were used to generate a de novo initial model in the EMAN2. The final 3D reconstruction, in the EMAN2, with a tetrahedron symmetry applied, resulted in a density map with resolution at 17 Å. This density map was calculated via the use of the 0.143 criterion of the Fourier shell correlation (FSC) curve with a mask. A Gaussian low-pass filter was applied to the final 3D maps that had been displayed in the Chimera UCSF software package.

Tilt-pair validation for the cryo-EM map was performed by collecting data at two goniometer angles, 0° and 10°, for each region of the grid. The test was performed using the e2tiltvalidate.py program in EMAN2. Additional details on the tilt-pair validation is provided in Table 1.

TABLE 1
Table S1. Tilt-pair validation of single stranded DNA tetrahedron

| | |
|---|---|
| # Total particle pairs | 49 |
| # Particle pairs in cluster | 15 |
| Fraction in cluster (%) | 30.6 |
| Mean tilt angle (°) | 9.47 |
| RMSD tilt angle (°) | 4.00 |
| Mean tilt axis (°) | 42.4 |
| RMSD tilt axis (°) | 54.9 |
| Experimental tilt angel (°) | 9.9 |

A total of 53, 75, and 46 micrographs of ssDNAs that folded into triangular prisms, pyramids, and pentagonal pyramids, respectively, were collected. Subsequently, 226, 330, and 238 particles were extracted, respectively. The initial models were generated as mentioned above and the final reconstructions were applied with corresponding C3, C4, and C5 symmetries that yielded EM density maps at resolutions of 32, 25, and 26 Å. These resolutions were calculated with the use of the 0.143 criterion of the Fourier shell correlation (FSC) curve with mask, respectively.

### Sequences

9 crossing number square knotted DNA (SEQ ID NO: 1):
ATCCAGGAAGGGCTATGGTTTTCATCGAAGATAGACAAATAGACA
GCATGCCAATGATGATCAGAAGAGGACGAGTTTTGGCCATATCTGGCATGTTTTT
CATGCCGATTCTATCTGAGTTCGCCAACCTACTTTTTGTAGGTCGAGGAGAGCTT
TTAGCTCATCGAACTCTTACCAGTCATCTTATTTCCCAGCAATAACGAGGTTGGG
TTTTAGGACTTGCTTCGACTAGGAACGGGAGGGAGAAGGGAACGAGATACTCGT
AGATTTTGTTGACCGAAACAAACCAAACCGCAGCTACGACGCACCATGATGGTA
TCTCGATTTTAGCTCAGGGCACTAGTGGTAGGTAGTGGTGGGGTGGCGAACTACC
TGTCTATCTTTTCCTCAAGACTAGAATCCTCATCGTGATGAGTACAGGAACAGTA
GGACAGCTGATTTTGGATTCCCAGAGTGACTTTTTGTCACTAGGCACCTCAGCGA
AATCTATCTCGGTTTTTCCGAGAACAGTACCAGTTTTAGGCTCGCGGTTCTTGGA
ACCTTGGCAGTAGACAACCTTTCCAGGGAACGTGCTTTTGACCTAACTTGGATGC
TTTTTGCATCCTGTTAGTAGCGGCCTCCGTGACGTAGTTTTTCTACGTAGCTGATG
GATTTTGTACCGCTGCAGTCTGCTACATCAGGGACGGACTGATTCACCTCTAGCT
CACATTTTCTAGCGGGTGGTGAGCTTTTTGCTCACGAGTGGAATTCAACGGCCCT
TTCAATCTTTTTGATTGAAGCATCCGTTGTTTTAATTCCACTCGCAGTACATCTAT
GTGCTCAGTCTCCGTTTTTCGGAGTAGTCGCACATAGATGTACTGCCACCCGCTA
GTGTGAGCTAGATCATGATCAGTCCGTCCCTGATGTAGGTTTGTGCAGCGGTACT
CCATCAGCTAGTAACACGCAAGTGGTACCTCCTGGCTTTTTGCCAGGCTAAGCCA
CTTGCGTGTTACTCACGGAGGCCTTTTGCTACTAACAGCTTCGTTTTTCGAAGCAA
GTTAGGTCGCACGTTCCCTCTCGTGGTTGTCTACTGCCAAGGTTCAGTCGACCGC
GAGCCTCTGGTACTGTTGAAACTTTTTGTTTCATAGATTTCGCTTTTTGAGGTGCC
TACACGATCTCATCAACGCTAAGGCCACTTTTTGTGGCTACCTCGTTGATGAGAT
CGTGTCTGGGAATCCTCAGCTGTCCTTATTGTCCTGTACTCAGATGAATGAGGAT
TCTTCGATTGAGGGATAGACAGGTCTGATGCCACCCCACCACTACCTACCTTGTC
TGCCCTGAGCTTCGAGATACCAGGTGAGTGCGTCGTAGCTGCGGTTTGCAGACTT
TCGGTCAACTCTACGAGTATGGAAATCCCTTCTCCCTCCCGTTCCTCAAGAAAGC
AAGTCCTCCCAACCTCGTACTGTCTGGGAAATAATCACGCTGGTAAGAGTAGTCT
GAGCTGCTCTCCTCGAGTAGCTCGTCGACTCTAGTCGGTGTGTTTTTCACACCTCA
GTGAGTCGACGAGCTACTTGGCGAACTCTTTTAGATAGAATCGCTCTCTTTTTGA
GAGCAGATATGGCCCTCGTCCTCTTAGTTCCATCATTGGCATGCTGTCTATACTA
GTATCTTCGATGCCATAGATGCTCCTGGAT
23 crossing number 3-square knotted DNA before hierarchical design
(SEQ ID NO: 2):
ATCACCCTTGGTCTCAGACGGATCAATCGCTGTGTTACTCGTACGG
GCGATTACAGATTCACTGCGACACCTGGGAGATGCCGACTCCCATGACCAACTTT
TTTCCACGTCATGGTCTTGTTTTCACCATACGTCCCCTGTTTTTTGTCGAGACGTTT
AGACTCAACGCCCGGCACTCACGACGTGAGAATGGGCCTCTACGTCCTGCTCGTC
GTCTACACATGTGCTACACCGTGTTTAGTTTTTTGGAGGCACGGTGCAGCTTTTAG
TACATGTGCCCAGAGTCTTTTGCCGTCGTTGTAGCAGCAATCATGTTGTCAAGCA
TGTAGCTTTTACGGAGAGATCCGTTCTTTTTTCGGTCGATCTGTTTCCGTACCTGC
GCTAGGCCGATGTGATGCCTGGCTCGTTACTTTGCACTGTCCGACAGGTTATTCC
GAGTTCGGTTTACATGTTTTTTGCCTGAAACCGACTCGTTTTAGTAACACGAATA
GGCTTTTTTGCGCTTTCGTCAGGAGGCAGTCTATGAACTGGCTTTGGGACCTACC
ATGCGGCTCCTGAAGCTGAACGACACGGACCTTCGGTCGGTGTAGATTGTCTCGG
TTGTCATCCATATGGCACGGAAAGACAGCGTGTTACCCACCGATCGTTCGAGCAG
CTCAACCGGATTGGAGGATCAGCTCGCTGGAGTTCTGGCGACGCACGACGCACC
TTTTAGGACAACACCGATTAGGCCTTAAAGCTACTTACGGTATCTGGGCATTGTG
GTCTACTTCCAGGTGTAGGTCCCTAATCCCGGCGTGCTACAAGCCCATGAACAGG
GCCTTAGGCGTCCACCTTCCCTTTGGAAGATTTCCAGCGCAGCGACACAGCGATC
CCGTGGTTAGTAGGTGATCTGTTGTCCGTGAGACGTACAAGGTATTACCACTCGA
CGAAAGTGACATATCCCCAGAAATGTTCTGCATTTAGTATCCCTGGATGCTTGCT
GTCTTCCATGTGCAATGTAAAACCGCTGGAGTTGGCTTTGGTGTCGTCAGGTAAG
CGTTTGGCAGAGGCAAGAGAGCAAATTCGCCATAAGAGAGATCTCGCGAGGTAT
GAGGGTACCTTGCGCTCTTAGCCATGGTGCACCTCACACTTCACTCTCTGGTGGT
TCGCAGAGGCCTGAGCTGTTCGCACGTCCGCTGCACCTCGTGACCACACTTGTTA
GGAATCGAATGGGACGACTAATGAGCCTTTGAAACTGGTTGACGTCTCCAGAGG
ACGTGTCTAAGGCTGAGGCAATGCTGGCTGACACGGACAACCTGGTCACTTGCG
CTCCGTCGTCACGACTCTTGGGGCCATGAGGTTGACGAGTTTTTTGCCTCCAACC
TGCAGCTTTTCAAGTTAAGTGAGACCTTTTTTCACAGCACTTCATCGCCAACTCGC
CAATGCGACAAGCTGTCTCTGGAAAACGCACATACGTTGGAGTGGATGGAGATT
CCGTAGACGTATGACTGTTTTTTCAGTGATACGTGATACTTTTATGGGTAGGAGA
AGTGGTTGAGTTCGTACCATTTGCCAGTCTCGTCTTCCTCCAGACCCTACGTTTTC
ATATGACGTCAGTACTTTTTTCGTGTGACGTGGTTTCCCCGACACTGGAGTCGCTT
TGGCAAGGGGTTGTGGCAAATCGTTAACGGTTTGCACGCAACATCTGCATCATGG
CAACCTTTTTTCGGTTCCATGAAAGAGTTTTCTGAGTGACCAGTCTAGTTTTTCTA
GACGAACTACTCAGCTCTTGCTCTTAACCGTTTTTTGGTTGAAGAGCTGCAGTTTT
ATGTTGCGTGCAAACCCGGGACGATTTGCCACAACCCCTTGCCAAAGCCCAACC
AGTGTCGGGTTTTGAAACCTGCCAACACGTTTTTTGTACTTGGCACATATGCGTA
GGGTCTGACTCAAGACGAGACTGGCAAATGGTACGAACTCAACGAGGAGTCCTA
CCCATGTATCCCCTCACACTGTTTTTTCAGTCTGAGGGCTACGTTTTGAATCTCCA
TACGCTGCAACGTATGTGCGTTTTCCAGAGACACGAGACCGCATTGGCGAGTTGT
TTTGCGATGGCCCACTGTGTTTTTTGGTCTTGGGCAACTTGGCTGCGAAAGCGAG
GCTTTTTTCTCGTGCTTTCCATGGTTTTCCCCAAGAGTTCGTTCGACGGAGCGCAC
CGCACCAGGTTGTCCGTGTCCAGTAGCATTGCCTCTTTTAGCCTTAGACGTCGGA
TCTGGAGACGTCAACCAGTTTCAAAGACATCGTAGTCGTCCCATTCGTTTTATTC
CTAACAGCCAGAGTCACGAGGTGCAGCTCCTCCGCGAACAGCTCGCTGCTCTGC
GAACCTTTTACCAGAGAGTCTCCTCTGAGGTGCACCATGGCTAAGAGCGCAAGG
TACGAGTATACCTCGCGATTTTGATCTCTCTTCCCTGTAATTTGCTCTCTTGCACG
CCGCAAACGCTTACCTGAACCTGGCAAAGTTTTCCAACTCCAGACGACGTACATT
GCACATGGAAGACAGCAAGCATCCAGAGTGACTAAATGCAGTTTTAACATTTCT
GGGGATACGCAACTTTCGTCGAGTGGTAATACCTTGTACCACACACGGACAACA
TTTTGATCACCTACTAACCAGTTAATCGCTGTGTCGCTGCGCTGGAAATCTTGACT
AGGGAAGGTGGTTTTACGCCTAAGGATGGCGTCATGGGCTTGTAGCCTCTGCGG
ATTAGGGACCTACCGACACAAGTATTTTGACCACAATGTGCGATTACCGTAAGTA
GCTTTAAGGCCTAATCGGTGACAGCCTAAAAGGTGCTTTTGTCGTGCGTCAGTGT
GACTCCAGCGAGCTGAGGACGTAATCCGGTTGAAGGCCTCGAACGATCTTTTGGT
GGGTAACCCACTCTCTTTCCGTGCCATATGGATGACAACGCTTGTAATCTACACC
GACCGTTTTAAGGTCCGTGCGTGACAGCTTCAGGAGAGTGATGGTAGGTCCCAA
AGCAGCCTCATAGACTGCTTTTCTCCTGGAGCCAGCGCTTTTTTGCCTAGGCTCGT
TACTCGAGTAATACCCAGGCTTTTTTCATGTGGTATTAACTCTTTTGGAATAACCT
ACGTCCCAGTGCAAAGTAACGAGCCAGGCATCGCTCATGCCTAGCGCAGGTACT
TTTGGAAACTTAGTGACCGTTTTTTGAACGACTAACTCCGTGCTACATGCTTGCTG
TCATGATTGCTGCTACAACGACGGCAAAAGACATCGCACACATGTACTGCTGCA
CACCTCCTCCTTTTTTCTAAAAGGTGTGTAGCTTTTACATGTGTAGCGGTTTAGCA
GGACGTAGAGGCCCATTCTCACGTCGTGGGATCCGGGCGTTGATTTTGTCTAACT
CCGTCGACTTTTTTCAGGGCGGAGATGGTGCAAGAATCCCTCGTGGTTTTTTGTTG
GAGGGATGAGTCTTTTGGCATCTCCCAGGTGTTGTCGTGAATCTGTAATCGCCCG
TACGAGTAAGTCTGCGATTGATCCTTTTGTCTGAAGTTCAGGGTGAT
23 crossing number 3-square knotted DNA after hierarchical design
(SEQ ID NO: 3):
ATCACCCTTGGTCTCAGACCAACTCGCCAATGCGACAAGCTGTCTC
TGGAAAACGCACATACGTTGGAGTGGATGGAGATTCGACTCCCATGACCAACTT
TTTTCCACGTCATGGTCTTGTTTTCACCATACGTCCCCTGTTTTTTGTCGAGACGTT
TAGACGCTACATGCTTGCTGTCATGATTGCTGCTACAACGACGGCAAAAGACATC
GCACACATGTACTGCTACACCGTGTTTAGTTTTTTGGAGGCACGGTGCAGCTTTT
AGAACTGAAGCGGTTTAGCAGGACGTAGAGGCCCATTCTCACGTCGTGGGATCC
GGGCGTTGATTTTACGGAGAGATCCGTTCTTTTTTCGGTCGATCTGTTTCCGTACC
TGCGCTTGGCCGATGTGATGCCTGGCTCGTTACTTTGCACTCTCCGACAGGTTATT
CCGAGTTCGGTTTACATGTTTTTTGCCTGAAACCGACTCGTTTTAGTAACACGAAT
AGGCTTTTTTGCGCTTTCGTCAGGAGTACTTCCAGGTGTAGGTCCCTAATCCCGG
CGTGCTACAAGCCCATGAACAGGGCCTTAGGCGTTGTTGTCCGTGAGACGTACA
AGGTATTACCACTCGACGAAAGTGACATATCCCCAGAAATGTTGATCGTTCGAGC
AGCTCAACCGGATTGGAGGATCAGCTCGCTGGAGTTCTGGCGACGCACGACCTG
CATTTAGTATCCCTGGATGCTTGCTGTCTTCCATGTGCAATGTAAAACCGCTGGA
GTTGGGCAGTCTATGAACTGGCTTTGGGACCTACCATGCGGCTCCTGAAGCTGAA
CGACACGGACCTTTCGCGAGGTATGAGGGTACCTTGCGCTCTTAGCCATGGTGCA
CCTCACACTTCACTCTCAGCTGAGGCGGTGTAGATTGTCTCGGTTGTCATCCATAT
GGCACGGAAAGACAGCGTGTTACCCACCGCACCTTTTAGGACAACACCGATTAG
GCCTTAAAGCTACTTACGGTATCTGGGCATTGTGGTCGAGGCAATGCTGGCTGAC
ACGGACAACCTGGTCACTTGCGCTCCGTCGTCACGACTCTTGGGGCCACCTTCCC
TTTGGAAGATTTCCAGCGCAGCGACACAGCGATCCCGTGGTTAGTAGGTGATCG
GTTCGCAGAGGCCTGAGCTGTTCGCACGTCCGCTGCACCTCGTGACCACACTTGT
TAGGAATCGAATGGGACGACTAATGAGCCTTTGAAACTGGTTGACGTCTCCAGA
GGACGTGTCTAAGGCTCTTTGGTGTCGTCAGGTAAGCGTTTGGCAGAGGCAAGA
GAGCAAATTCGCCATAAGAGAGATCCCATGAGGTTGACGAGTTTTTTGCCTCCAA
CCTGCAGCTTTTCAAGTTAAGTGAGACCTTTTTTCACAGCACTTCATCGCGGATC
AATCGCTGTGTTACTCGTACGGGCGATTACAGATTCACTGCGACACCTGGGAGAT
GCCCGTAGACGTATGACTGTTTTTTCAGTGATACGTGATACTTTTATGTTGCGTGC
AAACCCGGGACGATTTGCCACAACCCCTTGCCAAAGCCCAACCAGTGTCGGGTTT
TCATATGACGTCAGTACTTTTTTCGTGTGACGTGGTTTCCGTAGGGTCTGACTCAA
GACGAGACTGGCAAATGGTACGAACTCAACGAGGAGTCCTACCCATCTGCATCA
TGGCAACCTTTTTTCGGTTCCATGAAAGAGTTTTCTGAGTGACCAGTCTAGTTTTT
CTAGACGAACTACTCAGCTCTTGCTCTTAACCGTTTTTTGGTTGAAGAGCTGCAG
TTTTATGGGTAGGAGAAGTGGTTGAGTTCGTACCATTTGCCAGTCTCGTCTTCCTC
CAGACCCTACGTTTTGAAACCTGCCAACACGTTTTTTGTACTTGGCACATATGCC
CGACACTGGAGTCGCTTTGGCAAGGGGTTGTGGCAAATCGTTAACGGTTTGCACG
CAACATGTATCCCCTCACACTGTTTTTTCAGTCTGAGGGCTACGTTTTGGCATCTC
CCAGGTGTTGTCGTGAATCTGTAATCGCCCGTACGAGTAAGTCTGCGATTGATCC
TTTTGCGATGGCCCACTGTGTTTTTTGGTCTTGGGCAACTTGGCTGCGAAAGCGA
GGCTTTTTTCTCGTGCTTTCCATGGTTTTGATCTCTCTTCCCTGTAATTTGCTCTCT
TGCACGCCGCAAACGCTTACCTGAACCTGGCAAAGTTTTAGCCTTAGACACGTCC
GAGTGAGACGTCAACCAGTTTCAAAGGCTCATGCCACGTCCCATTCGTTTTATTC
CTAACAGCCAGAGTCACGAGGTGCAGCTCCTCCGCGAACAGCTCGCTGCTCTGC
GAACCTTTTGATCACCTACTAACCAGTTAATCGCTGTGTCGCTGCGCTGGAAATC
TTGACTAGGGAAGGTGGTTTTCCCCAAGAGTTCGTTCGACGGAGCGCACCGCACC
AGGTTGTCCGTGTCCAGTAGCATTGCCTCTTTTGACCACAATGTGCGATTACCGT
AAGTAGCTTTAAGGCCTAATCGGTGACAGCCTAAAAGGTGCTTTTGGTGGGTAAC
CCACTCTCTTTCCGTGCCATATGGATGACAACGCTTGTAATCTACACCGCCTCTTT
TAGCTGAGAGTCTCCTCTGAGGTGCACCATGGCTAAGAGCGCAAGGTACGAGTA
TACCTCGCGATTTTAAGGTCCGTGCGTGACAGCTTCAGGAGAGTGATGGTAGGTC
CCAAAGCAGCCTCATAGACTGCTTTTCCAACTCCAGACGACGTACATTGCACATG
GAAGACAGCAAGCATCCAGAGTGACTAAATGCAGTTTTGTCGTGCGTCAGTGTG
ACTCCAGCGAGCTGAGGACGTAATCCGGTTGAAGGCCTCGAACGATCTTTTAAC
ATTTCTGGGGATACGCAACTTTCGTCGAGTGGTAATACCTTGTACCACACACGGA
CAACATTTTACGCCTAAGGATGGCGTCATGGGCTTGTAGCCTCTGCGGATTAGGG
ACCTACCGACACAAGTATTTTCTCCTGGAGCCAGCGCTTTTTTGCCTAGGCTCGTT
ACTCGAGTAATACCCAGGCTTTTTTCATGTGGTATTAACTCTTTTGGAATAACCTG
TCGGATCTGGCAAAGTAACGAGCCAGGCATCACATCGTAGTAGCGCAGGTACTT
TTGGAAACTTAGTGACCGTTTTTTGAACGACTAACTCCGTTCAACGCCCGGCACT
CACGACGTGAGAATGGGCCTCTACGTCCTGCTCGTCGTCTTCAGTTCTGCTGCAC
ACCTCCTCCTTTTTTCTAAAAGGTGTGTAGCTTTTAGTACATGTGCCCAGAGTCTT
TTGCCGTCGTTGTAGCAGCAATCATGTTGTCAAGCATGTAGCTTTTGTCTAACTCC
GTCGACTTTTTTCAGGGCGGAGATGGTGCAAGAATCCCTCGTGGTTTTTTGTTGG
AGGGATGAGTCTTTTGAATCTCCATACGCTGCAACGTATGTGCGTTTTCCAGAGA
CACGAGACCGCATTGGCGAGTTGTTTTGTCTGAAGTTCAGGGTGAT
57 crossing number 9-square knotted DNA before hierarchical design (SEQ
ID NO: 4):
CAACTCCTCGATTCCCGCTTGTTTGCACTTGTATGTACATAGTGTCA
GATCGCTTACGCTTGCGTGGCGATCATCTAGTCGTCGTTTTTTGTTCCACTAGATA
GTATTTTCAGGCGTTGACTAGGCGCCCGAGGTATTTCAAGAAGACAACTGATTAA
GTGTGTTTTCAAGTCCAACTACTACTTTTTTGCGGAAGTTGTCCCTTCTCTACCGT
CTTGCTCAGTGTTCAGAGACTTTGCTGGTGAAGTGCCGCACCGCCTGCTGTTAAG
AGAGCTTTTTTGGGAGCTTAACGCAAATTTTCCACGTAAACTGTTGGTTTTTTGAC
TCAGTTTAGGACTGAGTCAAGCATGGTCGGTCCCACAGAATCCGGAAGCCCAGT
CAGAAAACATGGTAGCGCTTCCCCTGGGTTTTTTCAGAAGGGAAGCGCCATTTTG
CATATTTACCACTCGTGACTCGTAGGGAGGACGCCTAGTGAAGCGCGTCTCATTT
TCTCGAGGAGTGAGGGCTTTTTTCCAGGCACTCTGTGTCGTCCCGTTAAGGATGT
GAGTGTCATGGCGAGCACCAGACAGAGGCCGTGAACGCTTGAAGCGAGAGCCGT
TTTTTGACATCTCGCTGGTGCTTTTCACCTAGCAACTATCGTTTTTTCATGAGTTG
CCTCTGCTTGAGCATCAAGTCGTAATCGTCCATGTTAGGTGCGCTTCTAGAAGAC
CGGTGTCCAGTATGGGTGTGATCTCCTGTCTTGCCTGGTACCGCTTTCCAGGATC
GGGTACGGGACTCGTGCCCGGAACATCAGGGCAAATCGCGGTCTCATATAGGAC
TGTACCCACACACTTGTCTTCGCCGTTCGGAACTTTCGGAGTATACACAGTCTTGT
TGCGCTTTAACAGATCATCTGTCATAAGTAAGGGGTTGCACATATCCAAGATGGG
ATTGCAACGGTCAACCGGATATTACGTATTTTCCTGGGCTAGCGTCGGATTTCGG
CTTCCTTGGGACTGAACAGGGATCGTTTGGTGCTAGGCGCTGGCCTTTCACACGG
GCGTGTGCAGCACAGATCATCTAATCATATGTAACTTGACCCCGTTTGCCTCATC
AGTCCTCATGATGAGTAAGTAGCCATCTTCAGTAAATCTTCGTCTAACATAGGGT
ACATACTCATCATGGACACTTTCTTCCGAATGCCTGCTACCCGCACCTCAACCGC
CCTAAGACTATTGGTGCTTCTTCACTTCGTCTGAGTCAGCCTCTCCATCTGTCACT
CCAAGGGATAGCGGACGACCCCGAGTGTCTTGAATTGCATCTACGAAAACGTTC
GGACTACTTTTCTACGTCTGTTATCTCAGTCCTTGCCGACGTCGTAGGTCGTGATA
ACGTCCAAGTCGGGTTGCGACCAAGGCCAGACGGAAGGTGGATTAGTTGTTACT
TCGCCAGTGAGAGTTTGCCTAGCTTGGATCACTGACGTCTGATGTTAGCGTAATC
TAGATCACTGGGGTTCTGCCACAGTCCGGTGAGTACAGCACAGATCGTATACATC
ACGAACGTTTGTCCAGTCGCGGAACACTGAAATGAACTAACGCTGACATGTATG
ACAACCAACATGATTACACACGCTTGTGAGCGAGTCTCTGCATGAGGAGTGCTCC
ACAGTGAGTGACTACGCTGCCAGCTGCAGCGCGCGTTTGTTTCGGGTCCCTTCAG
CATCGAACTGATCCGCTAAACGTCTTCACAGGCAAGACGTCGCGAATAGCGGAA
CGATGCAGGTATCTTAGTCATGCGCAACCAACCACCACAGGTGTGCTACTACGAT
TCGTCCGTCGGCATTTAACGCTCCGCCGTTGGATCGATAAATGGTTTCTGGAATT
ATATAGCCGGTAGATCGGCCGAGACACTTACGTATAATGAGCCCACTTATCCTCT
TCTTGTGAACACTGCTTGCTATACTCTTGCACACCTCGGTCGCAGACGGTAAAGA
CTACTGAATACGGCGATAGCGTATGTGCGCATGACGCGCGGTGAACACATGGTG
CCAAACACTTTCTTCGTGACCCGAATTGAATAATCGCTTATACAACCTGAAAGTG
CCGTACGCATTCGCCGTGGGCTAAAGCGCGATCAAGTCTCACCCTTGGTAGTCGA
GTTGTCGGGCGTAGCAGCTCTCTGTCCTCGTCCCATTACTTTGCTTAGTGAATGGA
AGACTGTGGTAATGCTCCTCACGAGGATAGTCAACTGACCGTCATGCAGCTGAC
GACGTTTAACGATCCTGCATCTCTCCCAAGATAGTCAGATCCGTAACGAGACTTG
CAGGACTCGAATACCCTCTAACTATCCGCACTAGAGTGCCGGAGGCAGTTCGCCC
TTGTAGGATCGGAGGTAGTCAGTTGATAGAAGGGCTGATGGCTCGTCCTCGAAGT
TGAACTCCCTCAGCCACACTTGGTTCCTACAGCAGTTGCATCTGAAGAATCTGTC
GACACAGACATGCCTTCGGCGCTCGTCCCACTTATGCAAGCGCATTCGTCGAGGC
AAGGAGCTCACTTAGAGCGTCTGATGGTTTAGTACTGAGTGAGATATCGTCCCTT
CTAATGGACTTTGGTCCTGCGGTTATTTTGGGAGCGGCACATGACGCGCCAAGCA
CTCATACTTTTTTCGATCAGTGCTCGTACTTTTCTTATGCTTCGAGCCACGTTGCC
GCGCATATCAATTCTACCTAGGCATACCTGTTTTCTTAACAGCCACTGTGTTTTTT
GCGGCTGGCTGGTCAGGGCGACTCTGAGAATAGAAAGGTGCGTGTAATCATAGA
GTGAGAGCGGGTGTCTGTGGTCTCCGATATCTTTTTTGAGCGCGGAGAGTTTATT
TTATGCCTCGTGGGGCTCTTTTTTGTAGCCCACGTCAGATTGCTGACAGATCACGT
TGATTTCGTTCCAGTAAAGTGAAATGAACTCTGACCGGGAGTGCCAACGATACTT
TTTTGTAATGTTGGCGCATATTTTAAGCGCGTGACTGCATATCAGATGAGATGTT
AGGGAGGTGAGAAGTACTCACCTTTTGGCGTGCACGCCGTGGTTTTTTGATTAGC
GTGGTCGGGTGGCCGGAATGGTATGCCTAAACACCAGACATCTGAACTGTCTGG
TTCATAATGTGACACTTGTCTGGCTTTTTTCGTGAACAAGTTTGTATTTTACTAGG
GAGGATGGGCTTTTTGCCCACGGATCCTAGTTACAATGATCGTCACGTTTTTTGC
CAGCGATCAGTCACTTTTATTATGAACCGTAATCTTCAGATGTCTGGTGTTTAGG
AGCGTCATTCCGGCCATTTTCCCGACGGACTTAATCTTTTTTCCACGAGTCCCACG
CCGGTGAGTACTTAAGCTCTCCCTAACATCTCATCTGATTGTCCATCACGCGCTTT
ATGCAGTATGATTACTTTTTTGTATCCATACTACTCCTTTTCGGTCAGAGTAGCCC
ACACTTTACTGGAACGAAATCATGACCATCTGTCAGCATTTTATCTGAAAGTTGC
TACTTTTTTGAGCCAACTTAGGCATTAAACGACCGTCGCTCTTTTTTGATATACGG
TCCCACATTTTGACACCCGCTTGTGCACTATGATTACACGCACCTTTCATCTGTCA
GAGTCGCCTTTTCTGACCTAGGCGCCGCTTTTTTCACAGGCCTAGTTAAGCAGGT
ATGCCTGACGTGAATTGATATGCGCGGCAACGACTGTAGAAGCATAAGGTACGT
CTATCGATCGTTTTTTGTATGGATAGATGGCGTTTTCGTCATTCTTCGCTCCCAAA
ACCTAACCAGGACCAAAACGACTTAGAAGGGACTTTTGATATCTCACCTCTGTCT
AAACCATCAGACGCTCTAAACATCCTCCTTGCCTCTTTTGACGAATGCGACGAAG
TAAGTGGGACGAGCGCCGAAGAGGGCTCTGTGTCGACTTTTAGATTCCCTGGATG
CAACTGCCAGGCAAACCAAGTGTTCACAAGGGAGTTCAATTTTCTTCGAGGACTA
CAGTTCAGCCCTTCTATCAACTGACACGTCCCGATCCTACATTTTAGGGCGTGTG
GCCTCCGGCACAGTTCCGCGGATAGTTGACAAGTATTCGAGTCTTTTCTGCAACA
GCCGTTACGGATCAACGATTCTTGGGAGATCCCAAGGATCGTTAATTTTACGTCG
TCAGTGGACAGACGGTCAGTTGACTATCCTCAGCTTGAGCATTACCATTTTCAGT
CTTCCAGACTGGAAGCAAAGTAATGGGACGAGGGATCTGAGCTGCTACGTTTTC
CCGACGTCACGACTACCAAGATTAGAACTTGATCGCTGCACAGCCCACGGCGTTT
TAATGCGTACGTGAGACTCAGGTTGTATAAGCGATTATGGGCATCGGGTCACGAT
TTTAGAAAGCAGGTGGCACCATGTAAGTGTCGCGCGTCATTATGTCATACGCTAT
CTTTTGCCGTAACTGGTAGTCTTTACGTAGAACGACCGAGGTAACGTAGAGTATA
GCATTTTAGCAGTGTTCTGACTGAGAGGATAAGTGGGCTCATTACGACCAAGTGT
CTCGGTTTTCCGATCTACCACTTCAATAATTCCAGAAACCATTTATGAGCACAAC
GGCGGAGTTTTCGTTAAGCCTCGACGGACGAACGACTTTAGCACACCTACGACG
GTTGGTTGCGTTTTCATGACTAAGTACTGAGCATCGTTCCGCTATTCGCGAGAGG
ATGCCTGTGAAGTTTTACGTTTCTGTGATCAGTTCGAAGTGATAGGGACCCGACT
AACACGCGCGCTGCTTTTAGCTGGCAGCCGTAAGACTCACTGTGGAGCACTCCTC
CAAGTGAGACTCGCTCTTTTACAAGCGTGTAGACAGATGTTGGTTGTCATACATG
TCCATACTAGTTCATTTCTTTTAGTGTTCCGCTTCACTACAAACGTTCGTGATGTA
TACACAGAGTGCTGTACTCTTTTACCGGAAGCGGGCAGAACCCCTGCTGACTAGA
TTACGAACAAATCAGACGTCATTTTGTGATCCAAGGTCCTAAAACTCTCACTGGC
GAAGTAATGGTCAATCCACCTTCTTTTCGTCTGATGCTGGTCGCAACCTCGTAGG
GACGTTATCGTGGTCTACGACGTCGTTTTGCAAGGTTCAAGATAACAGACCGTCT
GAAGTAGTCCGGTGCATTTCGTAGATGTTTTCAATTCAAGAATACGTGGGTCGTC
CGCTATCCCTTGGCAACGCAGATGGAGAGTTTTGCTGACTCAGCTTGCATGAAGA
AGCACCAATAGTCTTGCATGGGTTGAGGTGCTTTTGGGTAGTGTTCATTCGGAAG
AGTTCACCCATGATGAGGCGCAACCCTATGTTATTTTGACGAAGATTATACCTAG
ATGGCTACTTACTCATCATCGTCTCTGATGAGGCATTTTAACGGGAACTAGTTAC
ATATGGGTGAGTGATCTGTGCGCTTTACGCCCGTGTGTTTTAAAGGCGTCTGCCT
AGCACCATGACTACCCTGTTCAGGATGCAGGAAGCCGAATTTTATCCGACGCTTC
ATTTGGAAAATACGTAATATCCGGTACGTGGTTGCAATCCCTTTTATCTTGGATA
CTCACTACCCCTTACTTATGACAGATGTATTCTTAAAGCGCAATTTTCAAGACAA
CTTATACTCCGAATCTAGTGAACGGCGAAAGAGGGTGTGTGGGTATTTTCAGTCC
TATAGCACTTCGCGATTTGCCCTGATGTTCCTCAATCGAGTCCCGTATTTTCCCGA
TTTCAGAAAGCGGTACTGTAGGAGACAGGAGAGGCTGCCCATACTGGATTTTCA
CCGGGTGCCTAGAAGCGCATAACCGATGGACGATTGTCCATTGATGCTCAATTTT
GCAGAGTTTCTTCATGTTTTTTCGATAAGAAATAGGTGGCACCGTGGTCATGTCTT
TTTTCGGCTGACCACTCAAGTTTTCGTTCACGGCTCAGTACTGGTGCTCGCCATGA
CACTCGTGAGCTTAACGGGACTTTTGACACATCCGTCCTGGTTTTTTGCCCTACGG
ACTCGAGTGAGACGCGCTCGTTGAGGCGTCCTCCCTACGAGTCAACGTATGTAAA
TATGCTGGCGAACAAGTTCTGTTTTTTCCCAGCTTGTTCGCTATTTTCCATGTTTTC
ACAAGAGGCTTCCGGATTCTGTGGGACTACGTATGCTTGACTCTTTTAGTCCTGA
GGAGAGTCTTTTTTCCAACTCCTCACGTGGTTTGCTACATCCTCCCTTTTTTGCTCT
GATGTAAGCAGTTTTGCGGTGCGGCGGCTATCCAGCAAAGTCTCTGAACACTCGA
TCAGACGGTAGAGTTTTAAGGGAGCCTATCCGCTTTTTTGTAGTTAGGCGACTTG
CACACTTAATCGACCATCTTCTTGAAATACCTCGGGCTAGGACTCAACGCCTGTA
CTACTGCAGGGAACTTTTTTCGACGCTGCAGTGATCTTTTGCCACGCAAGGTAGT
CCGATCTGACACTATGTACATAATGCAGCAAACAAGCGTTTTGGAATCATCCGGT
TGG
57 crossing number 9-square knotted DNA after hierarchical design (SEQ
ID NO: 5):
ATCACGACGCTGTTTCACGGTTAACTCCTCACGCTCCACAGACCAG
TACTTCCGACTTTTCAACCTTGTACCACTAGGAGTGTTTCCTCGAGTTAACCTTTT
TTCTCCAACTCGATGGTATTTTAAGATTGGTGTCTGGCACATAGCTGCTCCTAGTA
CCAAACCTTAGATTCCTACGTTCTATAGGTTTTCAGAGGCCAAGATCGGTTTTTTG
GGAGCTTGGCACCACGCTCATGTGTCGGGATCATAGGCTGACGATTACTCGGTAC
TTGCGATCATGACATAGAGATTGTTGTGATTCGCTGTCCTTTTTTGTTGGGCGAAT
ACAGGTTTTATACCACTGACGAAGGTTTTTTCGTGTGTCAGGCATAGGTCGTTCG
TAGGGTCCCCTATGCGTTGATAGAATTTGGTGTTCGGAACGTCAGAACAATTCGG
GAGCAGCGTCATGGACTTTTTTCGAAGTGACGCATGACTTTTGCGTACTGTTCCC
TCGCAAATATGCCAGGAGTGTGAACACATCCTGGTCACCCTCAGGGACCATTTTA
CGATGTCCTCAATTCTTTTTTGTCCCGAGGAGCTCCCCTTGCTCAATGGTGACGAA
AGTCATACCGTCCAGAGCCGTGTGGATGGCTACGGTCAGAGGAGGGATCTCTGT
CCTGGTATGGGTGGAAGTCCTCGTGAGAGTTGGGTGCGCGTACATTGCCAGACTT
CGGCAAGTTTAGCCTTAACTCGTCAGGTGCCAGACATCCTCTATTGTCCGACAAC
TGTAGTCTCAAAGGCGTCTGGCTGCACGGCTTATTACGACGACTCCGTCAATGGA
CTGCCTTGACAGTCGGTGGCATAGCGGTGTTATGAGTCAGCGTGAGGTTACTACG
ATACTACGAGGGACAGATGTTCTCTGTACATGTGCAAGTGGGTGCCATTTAAGCT
AGTAGCAGAGTGCGATGTGCACTGTGGACTTCTTCATGCTTGGACTTGTAAACCG
AGAGCTCACGACACCATTCAATCGGCGACAGAGCACACCGTCGAATGCATAGGA
CCTTGGCTTTTGTATCCTACGGTGCATAAATGCCAGAGATGATGTAGCTCTCCGA
TATGCGATGTGACACCGTACCAGCACGTACGTATCCCATGTACGGACCTTGATCG
CGCGCATAACCGTCCGCCCACTATGTCGGCTGCTGCTACTCCCTCGATTATGTAC
CCTAAAAGGCACGTAGCGTGAAGGGGCATCATTTGGTCCACAATGTGCTAATGTT
TGAAGCCATAGCAGGGCGGGTGTATGTATGCCGTGGTGAAAGCGTCACGCTGGG
TAGCAGCGATGGCCACCAAGCAGCGTGCATCAGGTCCAGAATAGCCTCGCAAAG
CCAGAATAGACACACCAAGTCCATGTCCAGTCTCGCAAAAGTAGTGAAGGCTTT
TGCTGGGTTTCGACCTTAGTTACTGGCAGCATGGATAGCACATGACGCCAACGTG
TGAAAGTACCAATGTCGACTGCGCGAGAGTTAGACTGAACGGCAGTACGTGCTA
TCTCCTGCAGCGGTGATTCAATCTCGGAGGAGTAGGCAGAGCGTCGAGGAATAG
TCACTGGCGATAATCTTGTATTCGAGCGCTGCCGCATGCGAATTAGTCACCGTGG
GTGTATCTACGTTGGCTGCAGCTCGCACGTCGGCTGACTCCGTACCGCTCTTCCG
AACTATCGACCATAACCTGCCGGCGAACTCCTTTGCTAGGCTCCGGAAGCATAGA
CAATAGCTTCGAGTACCCATGGCCTTGTAGCTGAAAGATGACTTGCCACAATTGG
AGGCTCGTTCCAATCTGTCTGCGATTCAAACCTCTTCCCAAATCTATACGGTTACC
TCGCAGGGCTCCTCATTTCCATGATTCATTCTACCCGCTAACCGTCCTTGCCTGGA
AATAACCTGTCCGTAGCAGGTAGACCGCTTGCCATCGTCAGCGTCCTCCGTGCGG
TAAACTTGTCACTGAGGGTTACCTTTGGTCAAGTTTCTGACCGATCAGTGACACC
CAGACCTTTCCACACCCCGAAGGCTAGGCAAGACTGTGGTATGGAAGTGTGGGT
GTCCCTGCCTACGCCTCATGTGGACGCACTCATTGACCGACTCGTGGTGCTCTAC
ATGTTCCACAGCAACGGAAGGGGACTCGATGGTGCGTGCTAGAGAGTTCATGTA
CGGAATCCATGTCTATCCTAGGTCGATTAGGCGTTGACGGTTGCTCGGGAGTCCT
CAAGTTCTATCTATATCGGGGCATCTGAGATACGGTGTCTATCCATATACTCCGG
TACCGATTAACAGGTTCGATATGCCCTCAAGACTGGGTTGTACAGAGCAGGCTG
GTCCATCTAGGCTAGTTATTGCGTCCGGATGGCTGCAGGCCACGCATGCCTACGC
TTAGTATGCCTGCAGTCTAAATGTTTTGTCCGGTCAGTGACACTTGACGAAGAAT
GAGTGGATTGTGCTATTAACACGTCGGCTACCTCACATTCTGTCTACCCTTTACTG
CCTCGAGCTCCGAGCCGGAACGACTACACAGTTTCTGAAAACTTCGAAACTCCAT
TTGAAACTGCGAGACGCTTGCACGTCTACGCATGGCTGTTCGTAACGGAGTGACC
GTCACACTACAGGGATACAGCAAGCCATTCGCGTTGCCAACAGCGTCTAACATG
TATTAGAGGAGTCGGATTTCTTATGTGTCGCGTGCGGACTCTCGATCTCGTTCCA
CTCTCTGGAAAACATTTGTCTTAGGGGTGTTGACCCGTTCGGTTGTCATTGAAAT
GACCCGACTCGCACTAACTGCGTATTGGTTTCTCCAAAATGTTGGGGTCACTCCA
AGGTGACGTCTCGTGCCGTGTTGCGCACCATTTCCTTCACGTTAGTACCACTTTCT
CTGGGGTAGCTGAGGGTACAATAGGTTTTTTGTAGCTGTACCATACGTTTTCATG
ACTGTGAATGTCTTTTTTCCTGATCACATGACGGTGCTGAACCGAGCCTTCATCA
ACGTCCTGAGTACTGCCTACAACAAGTTCTGGTCTGCTGGATTACCTGGGAGAGT
AGGTTTTTTGCCACTCTCCCTCGACTTTTGTTATCTTGACTAGGAAGCGAACGCCT
GGGTTGGTCGTCGATAGCACCACGCTACCAGACCTCTTTTGAGGTAGCTCCTACC
CTTTTTTGAACTGGAGCCAATTGCAACATGCTTCCGACTCATGGTCATCATTGAT
GGGTCATCAATACGTGTTGGACAGTAGATGCGTACCTGCAGCACGAGTTTTTTGC
AAGGCTGCAGACACTTTTATTCCCATCTCAGGACTTTTTTGTACCGAGATAATTGT
CTCATTGGTGCCACCATCACCGTCCACCAATGGCCCAGATGCGATTCCAAAGCTG
TGCTGTCCGACTATTGTCGTACACTTTTTTATACTCGACAAGGCAGTTTTCGAACG
GAACTCAGGGTCGGAACCATTATCCGTGCGGATACACGCAATGTGACACTACCA
GGATTTTCCTCTACCTAGCCTAGTTTTTTCCCTTCTAGGTGACACGGCATGTGACT
GAGGCAAAAGCGGTTTCTGCGATGGCTAATTTCGTCGAAGCCGATCAACTATTTT
CCAGAGAGGTACCTTTTTTGTGAGCTCTCTCTTGCTTTTGAGACTACAGCCTCTAG
TTTTTCTAGAGATCCCAGTCTCGCAAGCATCTCCTCACTTTTTTGGTACGAGATGG
GAAATTTTATAGTTGATCGGCTTCACTCAAATTAGCCATCGCAGAAACCGCTTTT
GAGCAAGTCACATGCCTTTTGTGTCAAGCCAAAGGGTTTTTTCTAGGTGGCTTAG
AGGTCCTGGTAGTGCGTGATTGCGTGTATCCGCACGGATAATGGTTGTGCCCCTG
AGTTCCGTTCGCTGCCCAGGATAGTATTTTTTTGTGTAATCCTGTAGTCTTTTGGA
CAGCACACGTTCAGAATCGCATCTGGGCCATTGGTGGACGGTGATTCACGCACC
AATGAGTTTTACAATTGACGTGGTACTTTTTTGTCCTACGTCGGGAATGTGTCCGT
CTACTTGCTTTTTTCTCGTTAGACGGGTACTTTTGCATCTACTGCATGCTACGTAT
TGATGACCCATCAATGATGACCATGACTTCGAAGCATGTTGTTTTCAATTGAGCA
GAGTTCTTTTTTGGGTACTGCTTACCTCGAGGTCTGGTAGTCGGGTGCTATCGACG
ACCAACCCAGGCGTTCGCTGTAGCATCAAGATAACGTCGACATCTGGTGGCTTTT
TTCCTACCAGATGAGGTATTTTATCCAGCAGAGTACGCCTTGTTGTAGGCAGTAC
TCAGGACGTTGATGACCAGTCGGTTCAGCATTTTCCGTCACTCACTCAGGTTTTTT
GACATGTGAGGTCATGCGTATATACTGGCTACTTTTTTCCTATCAGTATCTCAGTT
TTCTACCCTGACGAAAGTGGTACTAACGTCTCTGAAATGGTGCGCAACACTCACC
GAGACGTCACTTTTCTTGGACAAGCCCCAACATTTTGGAGATCGTAATACGCAGT
TAGTGCGCCAGGGGTCATTTCATTTTATGACAACCGGCTTCATCAACACCCCTAA
GACAAATGTTTTCATGCCCGTGGAACGAGATCGATTTTGAGTCCCAGTGCGACAC
ATAAGAAATCACTATCCTCTAATACATGTTATTGCCTGTTGGCAACTTTTGCGAAT
GGCTCTGACGATCCCTGTAGTGTGACGGTCACTCCGTTACGAGACCCCATGCGTA
GATTTTCGTGCAAGCGGTCATGAGTTTCAAATGGAGTTTCGAAGTTTTCAGAAAT
CCCGTAGTCGTTCCTTTTGGCTCGTCTGTCGAGGCAGTAAAGGGTTCGTAGAATG
TGAGGTAGCCGGTCCGTTAATAGCACTTTTAATCCAGGGTTTCTTCGTCAAGTGT
CAGCAGCCGGACAAAACATTTAGGGACCAGGCATACTATTTTAGCGTAGGCAAC
ACCCGCCTGCAGCCATCCGGACGCAATAACGCTCGAAGATGGACCAGCCTGTTTT
CTCTGTACAATGGCCACTTGAGGGCATCAGCAACCTGTTAATCGGTACGCATGTA
TATGGATATTTTGACACCGTATTCGGACTGCCCCGATATAGATAGAACTTGAGGG
TTAAGGAGCAACCGTCAACGTTTTCCTAATCGACTGCTACTAGACATGGATTCCG
TACATGAACTCTCTAGCCGACACCATCGAGTCTTTTCCCTTCCGTTATGGACGAA
CATGTAGAGCACTGGCTTTCGGTCAATGATCTGGTCCACATGAGTTTTGCGTAGG
CAGTCCTCGCCACACTTCCATACCCTCCGATTGCCTAGCCTCGCTGGTGTGGAAA
GTTTTGTCTGGGTGTCACTGAGCACTCAGAAACTTGACCAAAGGTAACCCTCACA
CGCAAGTTTACCGTTTTCACGGAGGACTGGTCGGATGGCAAGCGGTCTGGTACGT
ACGGACAGGTTATTCGAGCTCAAGGTTTTACGGTTAGCGCCGTACATGAATCATG
GAAATGAGGAGCCCTGGTCACAAACCGTATAGATTTGTTTTGGAAGAGGTTTGA
ATCAGGTACAGATTGGAACGAGCCTCCAATTGTGGGCGTTCATCTTTCAGTTTTC
TACAAGGCCATGGGTGACGGAAGCTATTGTCTATGCTTCCGGAGCCTCCTCAAGG
AGTTCGCTTTTCGGCAGGTTAGCTGACATAGTTCGGAAGAGCACCTGCGAGTCAG
CCGACGTGTCCAGGGCAGCTTTTCAACGTAGATTGCGTGACGGTGACTAATTCGC
ATGCGGCAGCTAGCCTATACAAGATTATCGCTTTTCAGTGACTATGGACACACGC
TCTGCCTACTCACAGTCGATTGAATCACTCGGGCAGGAGATAGTTTTCACGTACT
GCGCTTTGGTCTAACTCTCGCGCAGTCGACATTGGTACTTGGTGACGTTGGCGTC
TTTTATGTGCTATCTCCAACGCCAGTAACTAAGGTCGAAACCCAGCAAAAGCGTC
GACTACTTTTGCTTTTGAGACTGGACGCTGTGTTGGTGTGTCTATTCCACGAGTGC
GAGGCTATGTGCGACCTGATGCATTTTCGCTGCTTGGCCCAGTTCGCTGCTACCA
TCGGTGACGCTTTCACCACGCGGAACATACACCCGTTTTCCCTGCTATGAACGGG
AACATTAGCACATTGTGGACCAAATGCAGAGACTTCACGCTACGTGCTTTTCTTT
TACTCAACATAATCGAGGGAGTACTGACAGCCGACATAGTGGGCACTGGGTTAT
GCGCGTTTTCGATCAAGGTGGTAGAATGGGATACGTACGTGCTGGTACGGTCGA
GGTTCGCATATCGGAGAGTTTTCTACATCATCGATCAGATTTATGCACCGTAGGA
TACAAAAGCCAAGGTGAGCTGCATTCGACGTTTTGTGTGCGAGCTCGCCGATTGA
ATGGTGAGACGAGCTCTCGGTTTACAAACGTAAGCATGAAGATTTTAGTCCAGC
ACGCACATCGCACTCTGCTCGACGCTTAAATGGCACCCACGACGACATGTACAG
ATTTTGAACATCTGTTGTCGATAGTATCGTAGTAACCTCACGCTGACTCATAAGTT
GGCTATGCCACCTTTTGACTGTGTGAGCAGTCCATTGACGGAGAACCCGTAATAA
GCCGTGCAGAGTCACGCCTTTGAGTTTTACTACAGTTGCTCAGAAATAGAGGATG
TCTGGCACCTGACGAACTCCCGCTAAACTTGCCGAATTTTGTCTGGCAATCCAGA
AGCACCCAACTCTCACGAGGACTTCCACCCATAAGGCGACAGAGATCCTTTTCTC
CTCCAGACGTAGCCATCCACACGGGAAGGGACGGTATGACTTTCGGGCACATTG
AGCAAGTTTTGGGAGCAGCGTGGGACTTTTTTGAATTACGCTCATCGTTGGTCCC
TGAGCAACACCAGGATGTGTTCACACTCCTGGCATATTTGTCGACAAACAGTACG
CGTCATCACATGCTTCGTTTTTTGTCCACATGTGTGCTCTTTTCCGAATTGTTTGCT
GTTTCCGAACACCAAATTCTATCAACGCATAGGGACAGCTACGAACGACTTTTCT
ATGCAGTCGACACGTTTTTTCCTTCCGACTTGGTATCCTGTTCGTCACCAACTTTT
TTGGACATGACGACACAATTTTCAATCTCTATTCTCGCATCGCAAGTACCGAGTA
ATCGTCAGCCTATGACTGTGACACATGAGCTTTTGTGGTGGTCTCCTCCCTTTTTT
CCGATGAGACCCTCTGCCTATAGAACGGCTCAATCTAAGGTTTGGTACTAGGAGC
AGCTATGTCTGATCCACCAATCTTTACCACACCAGTGGAGTTTTTTGGTTACTGGT
GGGAAATTTTCACTCCTAGTGGTACAGCAGTGAAAAGTCGGAAGTACTGGTCTGT
GGACAAGGAGGAGTTAACTTTTCGTGAAGGGATGTCGTGAT
67 crossing number hexagonal knotted DNA (SEQ ID NO: 6):
ATCCAGGGAGTGGCGTACCGACACAAGAGTACGCCCCTAATGCAT
GGAGCCTCGTCTAGGATATTTTCTTCCCCAGGCCTATTCACAGTTTTTTGGTCTCA
TAGGCAGCTAGCACTGCTAGTGGGATGAGCTGGACACACTTGTGGTGAGGCAGT
GCGTCAACCTAGGCTTACGGGACTACCTACCTCCCTGGGAGGCTGTCTTCACGGA
TGCTGCTTTGGTCTAAGCCTTTTTTCCTGTCGACCAATGAATCGCACTTGGCCATG
CTTTCTGCATTTGGGCTAGGATAAGGACCATGCTCGTGCACGTACAGAGAAGACT
CTTTTTTGTCCATTCTCTTAGTCCGGTATGAGCTTATGGACGAAGCCCTAGGATCT
GTAAGGAGCATCGAGTAAATCGTTCGTGTGTGGCATAGCGACGTCAAGCAGGTG
TACCTGTTCGAGTTTTCCTGGAAGAGTTCCCGTTTTTTGGACCACTCTTATGGCGC
AACCATCGAAATGGGGCTGTAATCGCCTGGATGGAGCGATTCCCTTTAGGACCGT
CCACTGCAGTGGACTTTTTTCGAGTCTGCAGTCCGACAAAGGCAGGTCTGTTGGT
CTTCGGTATGAGCATCCGCAACCACATGTGCCCAGAAAGTAGGATCACTTCTCTG
CATGTACGTCTCAGCCAAGAGGAAGAAGCCAGAGACACTCATGTCTTTTTTGTAT
GCAGTGTCAACGACAAGCGGTTCATTGGGATTGAGTGTCTATGATTCGTTCATCT
AGGCTCGACCGAGAGTAAGCCAGATGCCTTTTTTCTTAGGTGGCTTTGTCGCAAA
TCACGGAACCAAAGGATGCATGATTAGCGGAGACTCTCTTCCGATTTGCAATCGG
AGGTTCTGATCGTACCACATGCCGACTTACAACCATGGTAACATGTTTCTCGCCA
GGTAGATCTTCGTACTTATCCCGATAGAGTTCTTCGTTTGAGCCTCTCATGAGTTC
TACACTCTTGTCACTTCGGTACAGAAGTCGTGGATACCTGATTGGTTAAGATCTT
CCACATGGCACGACAACAGAGGCTTGCGTGCCTGGAATCGTTAGTGATATTCCTA
CATTGCCCCATTCCTCCGCTAAGGGTACGGTACACCAGTAGGCATGTCAACTCAC
ACACACTGGAGTTTGGAACAACGAGAAATTGTAGGCAGCCATGGTAACGTTTAT
AATGTAACATTTCGGAGGAACAGACAGCTCATCGCCTTCACACGGTACCATGTCC
TCAATCCTGGGGACATCCTGACATGCTAGGATAAATCGGGCTCTACGACGAACTT
GGCCTAGGTTTATAACGCGTCTATATATCTCGGAAAATGAGATACCTAGGGGACC
CTACACTTTCTCTACACCAAGCATCTGGCCCTACATGAGTTTCACATGGGTTGGA
ATCTAATGGTTGGCAATCGCAGAACGTGCCTTTAACCTGGGAGTAACAGACCTAC
GATTAGCCATTATCCCCTAATCTGCCAACCATTTGACACGCTAGGGATAGATGCT
TAGCCCGAATCTTGCACTTGTGTTTCCATATGGCGGATCTTCGAAACTCATGGCC
ACCTTTCCCAACGCATTTGGATCTTTGTGCAACTCGAAGGGACAAAGGTTCCTAT
CAATCAGTGTAAACCGGTCGCTCATTTCCGCCTGGTGCTCGTGCGTTGCTTACCT
AATACACTCTAAGGAATTTGGAACAAGCTGGTGACGGACGGCTGAGGTCGTCCT
GTTTCGGGTAACTGCACACTAGCGTCGTATCGTAGGGTAAGTGGCTCAGGGGAC
GCATTTGCTGACGGTCAGAATATCGCGTTTAATCCAGTTGTCTCGACGATTTGCA
GGACCTTAATACGCTGCATGTCAACGGCTACGTTGATAGTGCTTCTTACGTTTCA
CTCTCCAGTAGCTACAGCTCGTGAATCCCCAACACTGAGCATTTAACGCAGACCG
TTAGATGTACTCACACAGTCCAACGCTTCACTAGAAAACAATTTTCTAGGTAACG
CGCGAGACTCTAATCTTGTCCACACGATCGGATTAATGCCGGAACTGCTGATACT
AGCCGTCCCTCTCGATGTGTGACTTCGGACGATCCTTATTGGATGCTATGGACTC
CGACTGCGTAGGATAGAAGATACCGGCTACATCCAGAGCCATATGAAGTGTCCG
TTGTGTCGGTTACGGTGTCCTAACGGTCTTGGGACCCGTATCCTGCTTGCAACAC
AGCTTCTTTCAAGGTTGCTCGTTTGAGTGGTCTCCCCAGGGTTATCAAGACAGTG
GCCATACTAGCGTTCCACTCGGTAAAATCCGACCTCGGACACTCAGCCTGAGACG
CAGAGAGAACTAGGTCGGCTGAAGCCTTTGGTGAGTAACGACCCTTACTCGGAG
CGTGTGTTCGAAGGAAAGTCATTGACAGAAGGCACGTTTTCTCAGGACCTAGTGT
TAGGTTACGGGCTATCACATGAACTGGCTTATTGAATCTTTCACCATACAACAGA
TGTGTATGTGCATCCTATCTAAAGCACTGTTTGTCCAAGATGAGGATCTAGTATC
GGTTCCTCCTCACACACTGGGACACTTCTGTTTGACCCAGACTGGTCCGAATGCT
ACATGTAAGCTCTTCATAGTCTTTTCCAGCGGCACTACTGTAGGAGCCCAGAGTC
ACAACCGCCACATGGACACCCGCTGTTGTACTCCACGATTGGCATGGTAAGTGA
AGTTCTAGGTCAGAGGGTCCTATTCACGTGCGATACTAAGTGATACGATAGCCAT
CGTGACTTGTTTGTGACTACCAACCACCACGTCCTACTCTTGAGGAAAGGATCAC
TTTCCTACCGTAGCGATCTACTACCCAGACCACACCGAAGGCAAGATCCCTTCTT
GTTTGATTGGCTAGCCATACTACGTGAAGTTGTGCGAAAGTGGGACGTCTACCTA
CCGTCTCATATTGTATCATAACCGAGCCTGACGTTCTGTGGTAGACACTTCGAAA
CCTTGCCTTTTTTGGTTGTGTTTCTCGTCTGTCGACCATCTACGAAGACTTCGTCG
CATGCTGTTCAAGATGAAAGTCGGAACGGACAACCACAGTGTCCTTTTTTCTGTG
ATGTGGTAGTACGTAGCTGCAGCTGTCAACTATCGGATTCTAAGCGCGAGTGTGC
TGCTGAGGTAGACGTTGACACCACCCGCCACACCATAGTCTCTGGATGAATGACC
CCGTGGGAAGTGTGACGTGGTTTTTTCTCTTGCACACTAGCCGTATAGCCCAAAT
CCTACAGTCTTTGTCTGGCTATATGCCAATTGCGACGAATGCTATCACAACATGG
ATGTTTTTTGAGTGGTGTTGGATGCGTGAACATTTCTACACACGTAGAGAAGGAC
GTGCACGGAGCAGTCTTGCACTGGGCACATGTGTGTAGTCTTGCTATCCAACACG
GGATGACTTGTGCTGCATCGTGGCACTCGTCTAGTTTTTCTAGACTCCACCCACG
ATGCAGTTTCACAAGAGCCACCGTGTTGGATAGCAAGACTAAGTGTGTGTGCTTT
CCAGTGCAAGTGATGGCCGTGCACGTCCTTCTCTACGGACCAAGAAATGTTCATT
TCGCATCATGACCCACTCTTTTTTCATCCAGTCATTGATAGCATTCTTTGTCGCAC
GACACATATAGCCAGACAAAGACTGACCACGTTGGGTTTCTATACGGCTTTCCAC
CAAGAGTTTTTTCCACGTGTGGAATCCCATTTCGGGGTCATTGCAAACGAGACTA
TGGTGTGGCGGGTGTCACAAACGTCTACCTTTTCAGCAGGTTGGTCGCGCTTAGA
ATCCGATAGTAAGATCCTGCATTTGCTACGTACTTGAGACTCACAGTTTTTTGGA
CACGTCTCATGTCCTTTGTTCCGACTTGTGCAATGAACAGCATGCGACGAAGTCC
CTAGAGATGGTCGACTTTAGACGACCTCTACAACCTTTTTTGGCAAGAGAGGGAA
GTGTCTACTTTCACAGATTAGGAGGCTCGGTTATGATACAATACTGTTAGGTAGT
TTGTAGACGTCCAGAGAGCGCACAACTTCCATGAGTATGTTGGTCCAATCCAAGA
AGGGATAGATGCTTCGGTGTGGTCTGGGTAGTCCCAATCTACGGTAGGGTGATTT
GGCTCTCAAGAGTAGGACGTGGTGGGATCCAGTCACCAAGTCACGATTCTGTTCG
TATGTGATAGTATCGCACAACTCAAGGACTTTCCTCTGACCTGCTGTATCACTTA
CCATGCCAATCGTGCGAACCAACAGCGGGTTTTGTCCATAAACGGGTTGTTGGCC
TGGGCTCCTATCTCGTTGCCGCTGGAGACTAATGCTCGCTTACATGTAGCATTCG
GACTCCATTGGGTCCAGAAGTGTCCGTCCTTGTGAGGAGGAACCGATACTAAGC
ATGCATCTTGGACCAGTGGACAGCATAGGATGCACATACACATCTCCGTAATGGT
GGATTCAATAAGAGCTGTCATGTGATAGCCCGTAACCTTAGCTGAGGTCCTGAGA
CGTGTGGTACGTCAATGACTTGATGTCGAACCTGTTCTCCGAGTAAGTTTGGTCG
TGTTCGACCAAAGGCTTCAGCCGACCTTACAGCCTCTGTTTCGTCTCAGGCAACT
CTTCCGAGGTCGGATTTTACCGATACCTACGCTAGTATGTTTGCCACTCCAGAGA
TAACGTGTGGGAGACCACTAGGGTCAGCAACCTTGGAAGCCACTGAGCAAGCAG
GATCTTTGTCCCATCGAGGTTAGGACACCTTTGTAACCTGTGAAACGGACACTTC
ATATGGCTCGTTTGCTAGCCTTTGGTATCTTCTCGTGGTCGCAGTCGGAGTCCATA
GCATTGTCGAAGGATCGTCCTTTGAAGTCTGGTCTCGAGAGGGACGGCTAGTATC
CCATCATCCGGTTTCATTAATCCGCGACCTTGGACAAGATTCACCTCTCGCTTCCA
ACCTAGATTGTTTTCTAAGCCCGCGTTGGACTGTGTGAGTACAATGTCCGGTCTG
CGTTTGCTCCAGCTAGGGGATTCACGAGCTGTAGCTCAGCTAGAGTGCGTAAGA
AGCATGCCTAACGTAGCCGTTGACATGCAGATCCCAAAGGTCCTGCTCGTCTCCT
AGACTGGATTAAACGCGATATTCGCGTAGTCAGCTGCGTCCCCTGTCATCCTTAC
CCTACGATACGACGCTCACACACAGTTACCCGCAGGAATCGTGCAGCCGTCCGT
AGAGAGCTTGGCGTTAATTCCTTAGATTTGTGTATCCCAAAAGCAACGCACGAGC
ACCAGGACCTCGTGAGCTTTGACCGGTTTATGTGTTTTGATAGGAACACGGGTCC
CTAGACCTTGCACGATCCAAATGCCACACGAAAGGTGGCCATGAGTTTCGTGAC
AGCGCCATATGGCACAATCATCTGATTCGGGCTAAGCATCTATCTTCGTCGTGTC
TGGTTGGCAGAACGTCGGATAATGGCTAATCGTAGGTTGAGACCTCCCAGGTTG
GCACTGTAGCCGATTGCCAACCATTAGATTCGCTATCATGTGCTCATGTAGGGGT
CTTTGCTTGCCTGAGAGAAAGTGTCAAACGCCCTATTTGGTATCTCATCGAGGTA
GATATATAGACGCGTTATAATTGGGGGCCAAGTTCGTTTTCGTAGGTGAAGATTT
ATCCTAGCATGTCAGGTCTAACCCAGGTTTATTGAGGACACCTTCTCGTGTGAAG
GCTCCTAGCTGTACACGCCTCCGTGTTACATTATGTGGCTTACCAGACTTGCCTAC
AATTCAGTAGTGTTCTTTCAAACTCCAGATCCGTTGAGTTGACATGCCTACTGGTT
CTGAGTACCCTTAGCTTTGGAGGATCCTAGCAATGTAGGAATATCACTAATCCAC
ACAGGCTTTACGCAAGCCTGACAGTTCGTGCCATGTGGAAGATCTTCGAAGATCA
GGTATCCTTTACGACTGGCTAACCGAACACTCAAGAGTGTAGGTGAATTGAGAG
GCTCCGAAGTGAGTGATCGGGATAAGTACGAAGATCGTGGAGGCGAGCATGTTA
CCATATAGCTAAGTCGGCATGTGGTACGATGCTACACTCCGATTGCTCGGACACT
TTTCTCCGCTAATACGTCATCCTGCTAGTCCGTGATTTGTTTCGACAACAGACCCT
AAGTTTTTTGGCATCGTCTGTACTCTCGGTCTTTGAGCCTCTTGCAACGAATCATA
GACACTCAATAGATCGGAACCTTTGCTTGTCGTTCCTCAGGCATACTTTTTTGACA
TGCTGAGGTCTGGTTTCTTCTTCCTCCCTTTCGAGACGTACATGCAGAGAAGTTTG
GTTACTTTCTGGGTTTCACATGCTTCGGCGGATGCTCATACCGAAGACACTGTCA
CCTGTTTCCTTTGTCGGTACCTCGACTCGTTTTTTGTCCACGAGGTAGGACGTTTG
TCCTAAAGGTGTGGACTCCATCCAGGCGATTACAGCTAGGATTCGATGGTTGTTT
CGCCATCTCCTTGGTCCTTTTTTCGGGAAAGGAGCCAGGAAAACTTTTCGAACAT
CAGAACCTGCTTGACGTCGCTATGCACGGATCGAACTTTGATTTACTCGTGAAGA
CTTACAGATCCTAGGGCTTCGCAGTCAAGCTCATACCTTTGGACTAGTTCCATGG
ACTTTTTTGAGTCTGGAACGTACGTGCACGTTTAGCATGCAGTGTATCCTAGCCC
AAATGCAGAAGATCCTGCCAATTTGTGCGATTCAGACTGTGACAGGTTTTTTGGC
TTAACAGTCAGCAGTTTCATCCGTGAACTTTAGCTCCCAGGGAGGTAGGTAGTCG
TTGTAGCCTAGGTTGTTTACGCACCTATCCACCACAAGTGTGTCCAGCTCCGTATT
CTAGCTTTAGTGCTAGCTTGAGTGGAGACCTTTTTTCTGTGACACTCACTGGGTTT
GAAGAAAATAGAGACAACGAGGCTCCATGCATTAGGGTGACCCTCTTGTGTCGT
TTGTACGCGTGGACCTGGAT
9 crossing number square knotted RNA (SEQ ID NO: 7):
GGGAGAGGAUCCAGAUGAUGUCUCUAUGGCCAAAAGUUGAAGGU
CCGACUACACGUUGCGUAACGGUAAGCACUCAUAUGAGUAUGACAGGUCAUA
GCAGUAAAACGUCUAUAGUCCAGCAGGAAACUGCUGGAUAGGUUGGGCAGUG
CAACUAGGUCGACGAAAGUCGAGUUGCCUAGUAUGAAAAGAACAGUCGUCUC
GUGAUGUCCAGCGAGUAGCAGUAUCUUAGAGAGUCGAUUCUCGCACUGGGUU
GAAAAUCCAAGUAACACUUUGCGAAAGCAAAGUGGGUAACUCAAGGUUUCAU
CUCCACCACGAAAGUGGUAUGUGUCUGCCUUAAAAUUGCGGUGCUUCAUAAG
UGAUCGAUCGUUACUGUCAGCUCCAGACCUUUACCACUGUGUCAGACAGAAAA
UGGAUACGGGUCGUUACUCACCGCUUUAGUAGUGAUCAGUACUUCUACUUAA
GACCACUAGAUGCUAAAAUCUGGCCCAAAAGAUUUGGAUUUGACCCAUGUCCU
UACUGAUUACUCAUUCUCGUCAUCCGAGUUCAAAACUACGUAGGAAUGCAUCA
UGCAGAACUACUGAGCGAUCCAAUAAUGGUUCAGGAGUGUGUAUAUCUAAAA
GCGUGUCUAGCAGGUGUGGCUGGGUACACGUCUCGAAUAGACGAGCGACGUU
GGGCACUAUACGGUAAAACUGUGACACUGUUGUCGGAAACGACAACAGACCUC
AAGAGAUUAGCAACGUGCACGGAAACGUGCGUCGCAAAGCUACAAAAUCUAG
UGCACCUUAGGAAUGGUCAUAAUAGAUGCCGUCCAGCUGUCAUGACACCUAGG
GUCACUCCAAAACUCUCUAACUGGUACGAGAAAUCGUACCACUGACCAAGGGA
ACUAGAGACAGUGUCCUUCUAGAAAUAGAAGGACACACAUGAACGUUCCAAA
ACUUGGUCAGUCUCCAGCGAAAGCUGGAGAGUUAGAGAGGGAGUGACCCUAG
GUUAAGUAGAAGCUGGACGGCAUCUAUUAUGACCAAGUAACGAGUGCACUAG
AGUAGCUUUGCGACAACGGGAAACCGUUACGUUGCUAAUCUAAAACUUGAGG
UCCAUGUAUCGAAAGAUACAUGGUGUCACAGACCGCUGACACACCAACGUCGC
UCGUGAGCUGACGACGUGUACCCAGCACUUAUGACUAGACACGCAGAUAUACA
CACUCCAUCGACUCUAUUGGAUCGCUCAGUAGUUCUGCAAUCACGAGUCCUAC
GUAGGAACUCGGAUGACGACAUACUCAAAUCAGUAAGGACAUGGGUCAAAUC
UGUAGUCGUUGGGCCAGAAGCAUCUAGUGGUCUGUCAUGACAGUACUGAUCA
CUACUAAAGCGGUGUUCCUAAGCCCGUAUCCACUGUUAUAGUGCGUGGUAAA
GGUCUGCUAUUCGAAGUAACGAUCGAUCCACACCUGAGCACCGCAAAAGGCAG
ACACAUGUGCAGAAAUGCACGGAGAUGAAACCUAAAAUGAGUUACCCCAACG
AAGAAAUUCGUUGGUUACUUGGACAACCCAGUGCGAGAUGAACCAUUCUAAG
AUACUGCUACUCGCUGGACUGAUGCAUACGACUGUUCCAUACUAGGCAACCGU
UCGAAAGAACGCCUAGUUGCACUGAAAACCCAACCUAGCCGUAGAGAAAUCUA
CGGCCUAUAGACGACUGCUAUGACCUGUGAAUGAGUUAUGAGUGCUUACCGU
UACGCAACGCAAAUCUUGACCUUCAACGGCCAGUUCAUGUUCAUCUGGAUCUU
CUCGAG
15 crossing number DNA tetrahedron (SEQ ID NO: 8):
ATCGGACCTTACATCAGAACACACGAGGTCCTAATGTGGTGCTTCG
TGTGGAGCCAGATTAGGCCTAAAATCTTGAACACGGGTCTCAGTAATTGATGATG
CTGTCTGAGAGCATGCCACAACTACGGGAGGGTAGGCCCCTTGACATCCTTTCTG
GGTTCACCAAATAGGGGATGTGTTAGGATGTCTCGGTTCTTAAGCTATTGGTCTT
CTTGATTGTCAGCCTGTCGTTCATAACAGACGAGTAACTGTGTTGGCACGGCACG
TCTGTTGTCGGAATCCTGCACAACATTCTGTTGACTTTCCCAAGACGACCTTCCA
GTGCTGCTAAATGTATCTAGCCTCAACTTGCAAGGCGCGTACCGCGAATCGAATG
CGTTTTCCATCTGGAAAATTGTGTGAAACGTTATGACTATTGCGCACGAAATGTA
GAGCTCAGACCTCACGTGCGTTCGTTTGACAATAGGTGGTCGTAGTTGCCATGAC
GTGGCACTGTCCGAATCGGACGTGCAGCCTGCATTAGCTGGCTTATTTAGATTTC
TGAAGCCGGCTGTTGGCAGTTCTCCCGATTACAATAGCCCAACAGTGCACGTGGG
CACGGCCTCTGGTAGGGCATGTGCGATTGTCGAAAGTGGGACCAAGCTCTCTCGT
CGGTCCGCCTGGAGGTTTGGACCCATAAGTTTGCGAGAAATCACTGAGCTCTCTT
TCTCAACACCCAGACATCCAACGTTACGTACGACAAGTCTTGCAGCCATAGTTTC
GTGGATCTCAGGGAGCCATTCAAATCCGACTACTGGGATAGAGTCTGGTTCTATT
AGACGTAGACCGGTGATGCCTATAAAGCAGGGGTAGTTGCGGCGACTGGAAGGC
TCGAAGTGCCAATTGATTTTCAGGCTGATCTAGTTTTTCTAGATCAGCCTGAAAA
TCAACGTTACCTTCGAGCCTTCCAGTCGCCGGGTGTACCCCTGCTTTATAGGCAT
CATTTCCGGTCTACGTCTAATACCGTCAGACTCTATCCCAGTAAGACGATTTGAA
TGGCTCCCTAGACTCCACGCTATGGCTGCAAGACTTGTCGTACGTGGTCGGGGAT
GTCTGGGTGTTGAGAAAGAGAACCTCATGATTTCTCGCCTTATGGGTCCAAACGA
AGACGCGGACCGACGAGAGCGAGACGTCCCACTTTCTCCCATCGCACATGCCCT
ACCAGAGGTTTCCGTGCCCACGTGCACTCACCGGCTATTGTAATCGGGAGAACGT
AACGCAGCCGGCTTCAGAAATCTAAATAAGTTTCCAGCTAATGCGCAGGACACG
TCCGATTCGGATGCCGTCACGTCATGGCAACTCGTCTGACCTATTGTCCGAACGC
ACGTGAGGTTGAGGTTCTACATTTCGTGCGCAATAGTCATCCGACCTCACACAAT
TTTCCAGATGGACGCATTCGATTCGCGGTACGCGCCTTCACACGTGAGGCTAGAT
ACATTTAGCATGTGTGGAAGGTCGTCTTGGGGTCAACAGAATGTTGTAGGCTGTT
CCGACAACAGACGCAGTGCGCCAACACAGTTACTACGACCTTATGAACGACAGG
CTTTTGACAATCAACTCCAGCAATAGCTTAAGAACAGCTTGATCCTAACACAGAC
ACTATTTGGTGAACCCAGGGATGTCAAGGGGCCTACCCTCGAACAGTTGTGGCAT
GCTCTCGTCGAGCATCATCAATTACTGGAGACCGTGTTCAAGTTTATTTTAGGCC
TAATCTGGCTCGCAAGTAAGCACCACATTAGGACCTCGGCACTTCTGATGTAAGG
TCCGAT
20 crossing number DNA pyramid (SEQ ID NO: 9):
ATCTTGACTGGAATAACTTGTCGATCCTCGTGTGGCGTTTGTCAGG
GTAGTACAGGTGCGGACTCAGGAACGATGTATCGCCATACACCAAGCTACAGTC
TCCAAAGGAAAAGCGTTGCTTAGTCACCCCGACGTTCGGACCATGATGCGGGTG
AAAATGCAACGTTCTCCGTAGTGATCTCGGTGAGCTTGGTACGCCAGTGAAGTGC
GACGGACTAGTCGGATCGTTTGTATCCACTTACCAGCAATAACAAAGTGACGAC
ATCAAAACGTGCGACCGCACAGACCAGTGTTCTGTCCGCTAATTGACGTACCACT
ACCTAATCGAAACATACCTGCTCGTATGTGCTCGTGGATGCTTGCGGAACTGTTA
TGGGCTTTCTCTTTAGCGGCGATGTCTCGGCTATGGAGTTTTGGAGTTCTGCACCG
GCCGGTGTGGTGTGGCAAAGATCCCATTTACTTCTCTGCCCAGTGGCTGTTATCA
AGCCGAGCGTCAACCCAAACTTAACTGGCAGTGCTAACTGCACTGGGTTCTCTCA
CTTAAATGGACCTCGGCTCGGGCAGCTGGGCCACTCGAATCTGCGCTCCCAGATA
AACTCGACCCATCACTTGCATAGGTGGGCTGTCGTGACTAGCATGTTATCCTCAA
GCTCGGACTCGGGATCCCGTGTTCCGCACAATTCGGGCTTGAGGGCACGTGTCCC
ATAGGTACCTACGCTGGAGAAAACAGATCTGCGAAGGACAGGCTATTAGCTTTA
GATCTCTGTGGCACAACGGGTTGCCATTGGAGCTGGTATAAGCATAGCTCGCATC
TAGGCCCAAGTCTCTTCTAGGTTCTTCCTCGCGTCGAGAGAAGTATAAATCGCAC
CCAATCCATAATACCCAACCCGGCATAAAGTCCTCAAAGGATTACTGCAACTGTT
ACTGCTGATTCTCGGAAATGTGACGGTAGTTACGTACGGTACCAGACCCTTGACA
ATTTCGATTGGGTCCGGGTTCTTATCTTGATCACACTTTTCATGATACCTATGTGT
ACACAGCCTGAGCCTTAACTAGTTTGACGGGAAAACACAATAGACGCACTGTTTT
CAGCGAAATAGGACCTGAGAGGACTTTGTCAAGCATGGCTGCTTTGAGCCGTTG
GGAATCAGTGTTTATGGACGGATCTTGCACCTGACGTCTCTCACCTAAAGGTTAT
CTAGTTTTTCTAGATAACCTTTAGGTACTCGACGTCAGGTGCAAGATCCGTTCGTT
GACACTGATTCCCAACGGCTCAAAGCATTTGCCATGGTCCACAAAGTCCTCTCAG
GTTACCTTTCGCTGAAAACAGTGTACGTATTGTGTTTTCCCGTCCTAGTTAAGGCT
CAGGTGGGAGACACATAGGTATCATGAAAAGTGTGCGAGCCATAAGAACCCGGA
CCCAATCGTTGTCAAGGGTCTGGTACCGTATGCGACTACCGTCACATTTCCCGTC
ATCAGCAGTAAGCTGGTCAGTAATCCTTTGAGTTTGACTTTATGCCGGGTTGGGT
ACAACGAATTGGGTGCGATTTATACTTCGAGTGACGCGAGGAAGAACCTAGAAG
TTTAGACTTGGGCCTAGATGTCCACTATGCTTATAGAGCAGCCAATGGCAACCCG
TGTAGTGACAGAGATCTGCTAATAGCCTGTCCTACTGCCATCTGTTTTCTCCAGG
ACGCTTACCTATGGGACACGCACTGGCAAGCCCGAATTGTGTTTCGGAACACGG
GATCCCGCAGGCGAGCTTGAGGATAACATGCTCTGTGTGACAGCCCACCTATGC
AAGTGATGGGTTTTCGAGTTTATCCTGTGTCGCAGATTCGAGTGGCCCAGCTGCC
ATCAAGGAGGTCCATTTAAGTGAGAGAACCCATTTGTGCAGTTAGCTCGCAGAG
TTAAGTTTGGGTTCGTAGGCGGCTTGATAACAGCTGCCCTGCAGAGAAGTTGGGA
TCTTTGCCACACCACACACCTCGGTGCAGAACTCCAAAACTCCATAGCGATCACA
TCGCCGCTAAAGAGGCCCATAACAGTTCCGCAAGCACGAGCGAGCACATACCCA
GCTGTATGTTTCGATTAGTGTGCCGTACGTCAATTAGCGTTTGACAGAACACTGG
TCTGCGTAGTCGCACGTTTTGATGTGAGAACTTTGTTATTCAGTTGAAGTGGATA
CCGATCCGACTACTTGGTCGCACTTCACTGGCGCCTAAAGCTCACCGAGATCACC
GTCGAGAACGTTGCATTTTCACCCGTTTCATCATGGTCCGAACGTCGGGACACAG
AAGCAACGCTTTTCCTTTGGACCTGGTAGCTTGGTGTATGGCGATACTTTATCGTT
CCTGAGTCCGCCGGCGTACTACCCTGACAAACGCCACACGAGCGAGGACAAGTT
ATTCCAGTCAAGAT
22 crossing number DNA triangular prism (SEQ ID NO: 10):
ATCCACAGTAGATGCGCTTTCCGCTGTCGAAGCGACTAGAATCGAT
AACCTCGTCGCCTAAATTCTAGCGATCTTTCTCGTGGGTATGTAAGCAAGGACCG
AGTCCATAGTTGAAATCGGACACTTCTAGTCCTCTGGGCATAAACAGAGTCGACT
GGTCTCGTACATACTGTCAAACGAATGCCTGTAGAGGATACCTCCACCTACGATA
CCATAAGGATAGACATTTTCTGTTACACCTATTCATCACCGGTTTGGCTACGTCC
GTGCAGCCGTTCGTGAGTAAGACCACATCTGACGCGGGAGAAAGCGCATGTGCC
AAGTACAGACCTAGCAAGCCGGACGGATGTAATTCTGCCTAGGAGCACCAGGCT
ACGATCGATTCGCGTTTATAGCCTCTTATCTCGAGCTCGTCTCGTCTGCCACGTAC
TTCCGCGCGGGTATATAGACACTTTGCCAACGTGCTGAGCGTTTCCATAACTAGG
ATATATGTGGGTTCCAGTAGAGAGGGACAAGTCCTGGTTGATGGTTTTATCGCCA
CTGGGTCCAGCGAACCACCGATCAGCAGGATCACATCTGCAGTAGACGCGAAAG
AGGTCGGGACTCCCGTATAAGTGCTAGGCACCAAACGCATTTAAGAATCCGGAC
GGTCTGCCGGATACTCGTTCACCGTAGCCAAAATGGCACCTGAGGCTCAAAACC
ACGACCGCGAACATAAGAATACTTACGCTGCCATGGGACGGGTATCCCTGACAC
TTAGGAGGCAGTCGATAAAGAAGTGCAGTGACTGCCTATCACCCTTGGTCTGCCC
GTCTGTTAGACTCTATATTCTGACTACCCAGTGGTCACGATACCGGTGCCAGCCT
ACGGGTCTGAAGCCAGGAGTCGATGCTGAATTGTCCTTTGCGATACAGATATGA
ACGACGTGCTAGGCGCTGTGGAGTCCTACTGTACTACGGGATGTATACGTGTGCC
TAGACAATAAAGGTAAGCTATGCTACCATCGCAGGACTATTCACTAACGTCTTTC
CTGAGCTACATACCGATCGATAGAGTTGCCTTTTAGTTTAGATGAACGTCACCTA
AATATCTTCCAGACAGGCCGAGAACTGGCACGCATCTTCGTATTTAGTCGACGGA
TGGGCTCCTTTGATCCTGTTGGATTACGACTATGGTACCAAAGAACGATCTCTAT
GAGATCCACTTTCTCCGTGAAGAAACAATCCCGCACGAGGCAACTACGTTTACCC
TGTTGGAGAGCACTTGCTGCCTGCGCGTCAAACCGTCAGAGGGTGGCATCTGAG
ACGTTGTACCGGATTGATGGGAGAGTAGCGCCCAGCCGAGTTCTAGTTTTTCTAG
AACTCGGCTGGGCCACGCTCTCCCATCAATCCGGTACAGTTTGGCAGATGCCACC
CTCTGACGGTTTGACTTTGCGCAGGCAGCAAGTGCAGGTCAACAGGGTAAACGT
AGGCATCTCGTGCGGGATTGTTTCGAGACGGAGGTGGATCTCATAGAGACCATG
GTTTGGTACCATAGTCGTAATCCAACGTCGTGAAAGGAGCCCATCCGTCGACTTA
CGAAGATGCTGTGCAGTTCTCGGCCTGTCTCTGCGATATTTAGGTGACGTTGGTG
TAAACTAAAAGGCAACTCTATCTTTGATCGGTATGTAGCTCAGGAACCAAACTAG
TGAATAGTCCTGCGATGGCGTGATAGCTTACCTTTATTGTCTAGTTTGCACACGTA
TCGCGGACGTAGTACAGTAGGAAGACGAAGCGCCTAGCAGAGGGTTCATATCTG
TATCGCGGACAATTCAGCATCTCGCGTTGGCTTCAGACCCGTTCCTGCGCACCGG
TATCGTGATGGACCGGTAGTCAGAATATAGTTTAGTCTAACAGACGGGCATGATA
AGGGTGATAGGCAGTCACTGTCCCAGTTTATCGACTGCCTCCTAAGTGTCAGTTT
GGATACCCGTCTCGTTCCAGCGTAAGTATTCTTATGTTCGCGAGGATCGTTTTGA
GCCTCAGGTGCCATTTTGGTTTCTACGGTGAACGAGTATCCGGACACGGGTCCGG
ATTCTTAAATGCGTTTGGTAGGAGCCACTTATACGGGAGTCTTTCCGACCTCTTG
ACTCCCTACTGCAGATGTGAAGGCTGTGATCGGTGGTTCGCCCACTGCAGTGGCG
ATACCATCAACCAGGACTTGTCCCTGCGAACTGGAACCCACATATATCCTAGTTA
TCAGCACGCTCAGCACGTTGGCGTGTCTATATACCCGACATCCAGTACGTGGCAG
ACGCTCCACGCTCGAGATAACGTCCTATAAACGCGAATCGATCGTATTTGCCTGG
TGCTCGCTCCTAGAATTACATCCGTCCGGCTTGCTACCGTGTTACTTGGCACATGC
GCTTTCTCCCGCTTTGTCAGAGTGCGTCTTACTCACGAACGGGGAAACGGACGTA
GCCAAACCCATCATGAATAGGTGTAACAGATGTCTATCCTTATGGTATCGTTCTC
GGAGGTATCCTCTACAGTTGCTCGTTTGACAGTATGTACTTCACCAGTCGACTTTT
CTGTTTATGCCCAGAGGACTACTGGGATCCGATTTCAACTATGGACTCATCACTT
GCTTACATACCCACGAGAATTTAGATCGCTAGAATTTAGCTCTCGAGGTTATCGA
TTCTAGTCGCTTCGAGGAAGGAAAGCGCATCTACTGTGGAT
25 crossing number DNA pentagonal pyramid (SEQ ID NO: 11):
ATCATGCGTGAGCCGGACTCCTGTACTCATTGCTAATGTACCTATG
GCTAAGGAGTCGGGTACACATATCTCTTTCCCGATAAACACATCTGCGATTCGGA
AGCCCGTCAGCTTTCGATCGCTCTGATCACACCGGAGTTGGCTCTTGCTTCGTAT
AGTGCAGAAAGTGCGAATTGAGTAACCTCTCTCACGAATGAGAAGACACTACTG
CGTGTCGTCAGTTGGGATTTCCGCTGGGTACACAGCTTCGGGTACGCTCTATTCA
CTGCCCATACGCGGCTAGTGCCTGGAAAGCAATTCAAACGTCACGGAGTCTCCTC
ACGTGGGATGAGCACCCACGTGACTAACGAACCACTGGGACGCGGCTCGACTTT
GAAGTTTCATCTTTAAGCCCAGTAAACCGCAGCTTCAAACGAAATGTTACACGAC
ACATCCATCGCTCATGAACAAACGCATTTGTACTCCACTAGACCGGATCCTTACT
TTTCCGACGGTTTCCAATTGCCTGCCAAGACACAATCTAACGTCCTCGGAACCCT
TAGCGACGCACATAGGTCCCTCGTGGGACCAACCCGAAAGGAGTATGGAGAGTG
TCTTTTGGTGGGATTTAGGACCTCGCGTCCACACATCTGCATTTGTACCGCGGGT
GTAAGTCACAGGGCTTTTCGGGATCACTTGCTAACTTTTCCCTATTGCTATTACGA
ACAGGCAGACATATGAAAGGCCACCAGTCGGCATCCGGTTGCTGCACGTCACTT
GCTTTCCGGTTGTTCTCCTGGTAACAACATGTTGGCTCGTGAGTATGCAACTGTCC
AACTGTCCAGTAAAGATCTTCTGATGCCAAATCGTTCCTGGACTTCATTCAGTTG
TATCAGTACTTACCTGGTACGTTTGTTGAAAACAAGCCTTATGCACACTTTACCC
AGTTACGAACGGTCATGACTCTCCGTTAAGAGAGAAGAGTGATTTGCATCGGAA
ATGGACGTTTAAGGACCTTAGAGTAGTAAGCCATAAGACAGAGAACGAGAGGA
ATGGAGCTGAGCCGACATATTCCACTGACAAGCAATGCCAGCCGTGTGCTGCGG
CAATAGTTAACTCAGCTCATGCTACACTGGCCTCTTGATTAAACCTCTGACAAAA
GCCGCACGGACTGGGCACAGTAGCCGTAGCGTGTGATGTTCGACTGTGCACCAG
AGCTTTTGGTAACGCTTTTAGGTAGACGGGAACCGGGAAAACTGTGTGACATGTT
AACCAATCTGCCATATACGAGGAACGTCCCGAAGTGACTTTGCAGAACATCATA
CAGCTCCATGACTGGCACGTCCGCGAAGTCGGTTCGACGCACCTTGGTTGGTTTC
CGTCCCTTATAATGTGGTGGAGTACCAGTAGCAATAAGTCGGGTTCCTAGGCTCG
CAGAGTTCTATCCATGTGCCGACTATGGGACCGTCTCAGCAGGGAGTAGGTACG
AGACCCTGACCCTCGGGCAGTGGGAATCTGCGTTCTCTAGTTTTTCTAGAGAACG
CAGATTCATGTTGCCCGAGGGTCAGGGTCTCGTACCTAGGTTCTGCTGAGACGGT
CCCATAGTCTTTGGCACATGGATAGAACTAGTGGAGCCTAGGAACCCGACTTATG
ATCCATGGTACTCCACCACATTATAAGGGACTTTGGAAACCAACCAAGGTGGTGT
GAACCGACTTCGCGGACAGGTCAGTCATGGAGCTGTATCTGCTTCTGCGTCACTT
CGGGACGTTCCTCGTATATCACTGGTTGGTTAACATGTCACACAGTTTTCGTTGC
ACCCGTCTACCTAGCGTTACCAAAAGCCAGGCAGCACAGTCGAATGGGACACGC
TACGGCTACTGCCGACAGTCCGTGCGGCTTTTGTCAGTTTAGGTTTAATCAAGAG
GCCAGTTGGATCTGAGCTGAGTTAACTATTGCCCACTCACACGGCTGGCATTGCT
TGTCTTTAGTGGAATATGTCGGCTAGTGTCCATTCCTCTACGTGGCTGTCTTATGG
CTTAGAGGAGTAAGGTCCTTCGTCCATTTCCGATGCAAATCAGGCATCTCTCTTA
ACACCGTCTCATGACCGTTCGTACGGTCTTAAAGTGTGCATAAGTTTGCTTGTTTT
CAACAAACCGCTCAGGTAAGTACTGATACAACTGAATGACTTGCAGGAACGATT
TGGCATCAGAATTTGATCTTTACTCTCTCCTTGGACAGTTGCATATGGTCCAGCCA
ACATGTTGTTTGCGTCAGAACAACCGGGCAAGTGACGTGCAGCTGCAACATGCC
GACTGGTGGCCTTTCATATGCCAGTGTGTTCGTAATAGCAATAGGGAGTTAGCAA
GTGATCCCGAAAAGCCCTTCAGAGTACACCCGCGGTACAAATGCACCGATGTGG
ACGCGAGGTCCTTCCCACCAAAAGACAGGACAGATACTCCTTTCGGGTCTCACGC
ACGAGGGACCTATGACCAGGGCTAAGGGTTCCGAGGTTTACGTTAGATTGTGTCT
TCTCTGGCAATTGGAAGGAGAGGGAAAAGTAAGGATCACTGGGAGTGGAGTACT
GCGTTTGTTCATGAGCGATGGCCACGTCGTGTAACATTTCGTTTGAAGCTGCCTC
CTACTGGGCTTAAAGATGCTTCAAAGTCGAGCCGCGTCCCCAGCGTTCGTTAGTC
CGTTCTGTGCTCATCCCACGTCTACTCACTCCGTGACGTTTGTTTAATTGCTTTCT
CTGGTCTAGCCGCGTACATCCAGTGAATAGAGCGTACTGCCAGCTGTGTACCCAG
CGGTCCCAACTGACGACACGCAGTACGTCCTTCTCATTCGTGAGAGGTGCTACTC
AATTCGCACTTTGATGACTATACGAAGTTTCAAGAGCCAACTCCGGTGTGAGTGA
CTCGATCGAAAGCTGACGGGCTTGATGATCGCAGATGTGTTTATCGGGATTTAAG
AGATATGTGTACCCCAAGCCTTAGCCATAGGTACATTAGCAATGAAGCGAGGAG
TCCGGCTCACGCATGAT

### Topological Control Strands

1 (SEQ ID NO: 12):
GAACAGGTGAGCTCATAATGGCGTACGTTCGTACCCATTTTCGTAGACAC
TCCTCAGTTTTTTCAAGAGAGTGGGCGTGTTGAGACTACAACAGGTTTTT
TGTCACTGTAGTAGATCTTTTCCTGGCTTAAATCAGGTCGCCGGCATCTG
ATACTGGCATCAGGCTGTGACGGACAAAATCAACTTTTGACAAAGAGCAC
AGGGTTTTTTGATACTGCTCGAGCTCTCGGGAAGCGAGTTGGTTTTTTGA
AGTTCGCTTTCGGGTTTTGCAAGATAAGAGGCACCCTAGCCTCAGCGCAG
CAATTATTCGTTGTTGACGAAACGCAGTCCGTTTTCTCCAAGGTACATAG
GTTTTTTGCAACGTACCCATGGTCCTTGACATGTTTAGGTTTTTTGCGTG
ACATGTGTCGGTTTTAAGAGTGGTGGACACGGACGTCACCTTGAAGTCTG
ATGCACAACTCTGGACCCATGTGTATCATTTTAGATACGAGCAATCCGTT
TTTTGAGGGTGCTCGCCCGTGGAAGAGACAGTGCGGTTTTTTCTCTCCTG
TCTTGGCATTTTGGACTTCTCGTGCTTCCACAATGACC
2 (SEQ ID NO: 13):
CACCTGTTCGGTCATTGTGACACGTCGAGAAGTCCTGCCACTGTACGAGA
GTTTTTTCCGCAGTACAGCTTCCTTTTACGGGCCTTCTCCCTCTTTTTTC
GGATAGAAGGTATCTTGATACACATGGGTCGTTCGATGTGCATCAGACTT
CAAGGTGACGTCGAATTGCACCACTCTTCCGACTACACGCACGCTTTTTT
CCTAACGTGTACAAGGTTTTACCATGCCAGGGTTGCTTTTTTCCTATCCT
GGTTGGAGCGGACTGCGTTTCGTGCTGGCCGAATAATTGCTGCGCTGAGG
CTAGGCGTTGGCTTATCTTGCCCCGATGGATAACTTCTTTTTTCCAACTA
TCCACCCGATTTTGAGCTCTCTGCGTATCTTTTTTCCCTGGCAGATTTGT
CGTTGATTTTGTCCGTAGTCCGCTGATGCCAGTATCAGATGCCGGCGAGT
CACGTTAAGCCAGGGATCTCGATTGGTGACTTTTTTCCTGTCAATCGCTC
AATTTTCACGCCACCAGTCTTGTTTTTTCTGAGCTGGTTCTACGTGGGTA
CGAACGTACAGACAGATGAGCT
3 (SEQ ID NO: 14):
CATGACGGAGATTACCTCGAACTCCAGCTCGGATAGGTTTTGAGGACTAC
AACGTGTAATATTTTGCCTAAGGGTTAGCAATCCTGTCTAGCTAAAACAC
GTAGTTTTCGAGCTCTGACGTGACGCCATCGATACCTCAGCGTATCGCCT
CGGACTCTACCACAGAGGGTATTTTCCTTATGCGCCCAACGGTGTGTGGC
TCCGTGCACCAATTCCTGCCAGCGTTGCTTACAGCGACTTTTGCGCTCGA
CTCAATTCTCCACTGATC
4 (SEQ ID NO: 15):
TCCGTCATGGATCAGTGGACGTGTCAGTCGAGCGCGTCGCTGTAAGCAAC
CAACAAAGGAATTGGTGCACGGAGCCACACACGTGCCTGCGCATAAGGTA
CCCTCTGTGGTAGCACAGCAGGCGATACGCTGAGGTATCGATGGCCCTGA
TTCAGAGCTCGCTACGTGTTTTAGCTGCCATTGATTGCTAACCCTTAGGC
AAAATATTGAAGCATGTAGTCCTCCCTATCCGAGCTGGACAGAGTGGTAA
TC

### Example 2

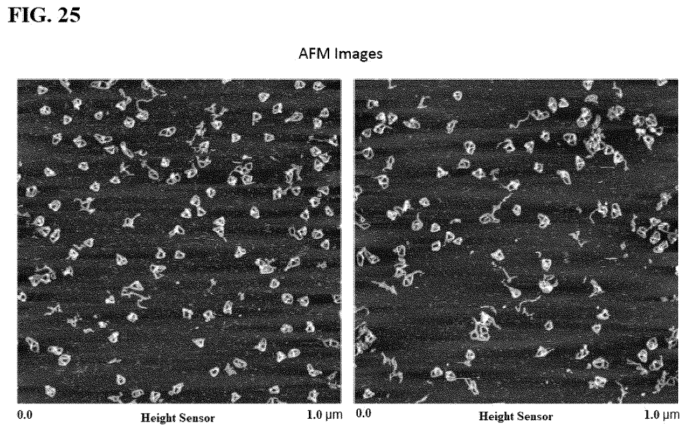

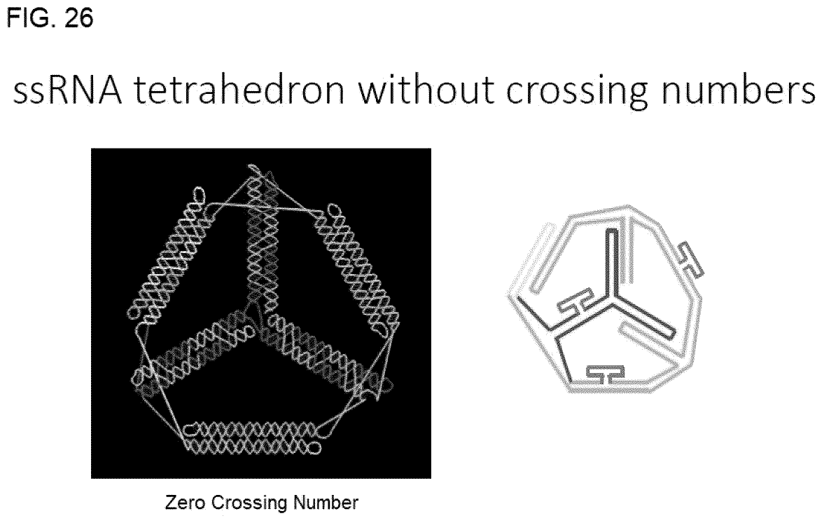

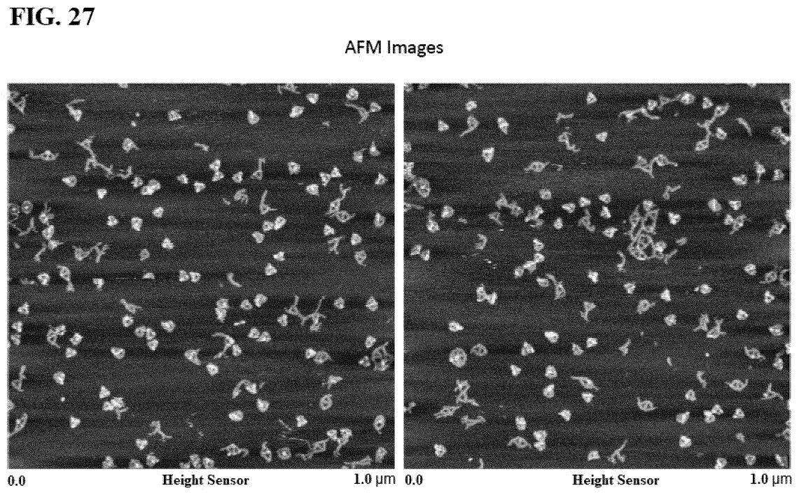

A comparison of single-stranded RNA tetrahedrons with or without knots was performed, including a comparison of their sequences, as shown in FIGS. 24-27. V. Kocar et al., Design principles for rapid folding of knotted DNA nanostructures. Nat Commun 7, (2016) refers to the construction of a significantly different form of knot than the knots of the present disclosure. The fundamental difference is the design of knot: Kocar et al. used the 2 single-stranded DNA on edge and adopted the natural double helix of DNA as knots. In the present work, in contrast, the knots are comprised of paranemic cohesion crossovers based on 4 single-stranded DNA. Kocar et al. failed to characterize their constructs and thus failed to confirm any structural features of the knots. In contrast, the RNA tetrahedron knotted nanostructures of this disclosure were characterized by AFM images to confirm their formation. The knots of the present disclosure are considerably larger and more complex than those referred to in Kocar et al., owing to the use of paranemic cohesion crossovers as the linking mechanism.

### Example 3

Target tumor cell killing by knotted ssRNA nanostructures comprising an antibody to the tumor target is demonstrated as described below.

Programmed Death-Ligand-1 (PD-L1) antibody is obtained from a commercial vendor and chemically linked to a knotted ssRNA nanostructure by the methods described herein.

The yield of knotted ssRNA nanostructures conjugated to antibody is monitored by the different mobilities of unconjugated and conjugated ssRNA nanostructures in an SDS-PAGE gel. The mobility shift demonstrates that knotted ssRNA nanostructures comprising an antibody are generated.

MDA-MB-231 breast cells positive for (PD-L1) are incubated with anti-PD-L1 knotted ssRNA nanostructures for 12 hours. Knotted ssRNA nanostructures conjugated to the anti-PD-L1 antibody are bound to their cognate ligands on cancer cells, initiating tumor necrosis.

### Example 4

Cancer cell killing by engineered T cells in a mouse model is demonstrated as discussed below.

The NSG mouse model is used to assess the in vivo anti-tumor effect of control ssRNA sequences not bearing an anti-PD-L1 antibody, as well as knotted ssRNA nanostructures comprising the anti-PD-L1 antibody. Alternatively, PTK7 siRNA are hybridized to ss RNA nanostructures. In another embodiment, anti-PTK7 antibodies are conjugated to ssRNA nanostructures. The CCRF-CEM cell line is used, which expresses PD-L1 and the PTK7 tumor marker, to monitor tumor growth in vivo. 8-10-week-old male and female NSG mice are injected intraperitoneally with CCRF-CEM cancer cells resuspended in Matrigel (BD Biosciences). Tumors are induced by subcutaneous injection of 5×10\hat{ }6 tumor cells, and mice are treated by IV injection of compositions comprising knotted ssRNA nanostructures comprising the anti-PD-L1 antibody. Tumor growth and condition of mice are monitored every other day. For antitumoral efficacy, 6-8 mice per group are used.

The knotted ssRNA nanostructures comprising the anti-PD-L1 antibody is generated using the methods described herein. The knotted ssRNA nanostructures comprising the anti-PD-L1 antibodies (or PTK7 antibodies or PTK7siRNA) are used to eliminate CCFR-CEM cells in an NSG humanized mouse model. Mice are then injected with PD-L1 positive CCRF-CEM cells (Day 0). A pharmaceutical composition in the form of a saline solution comprising the knotted ssRNA nanostructures comprising the anti-PD-L1 antibody is injected at Day 8 to the cohort test mice. T cell survival and CCRF-CEM cells is analyzed by flow cytometry at day 10.

In addition, peripheral blood from adoptive transferred mice is collected for analysis. Serum is used to quantify different cytokine release by ELISA.

The results show that the knotted ssRNA nanostructures comprising the anti-PD-L1 antibody traffic to solid tumor area are used to successfully reduce tumor volume and prolong survival mouse life. Mouse survival of each cohort is also measured, and demonstrates that the mice cohort treated with the knotted ssRNA nanostructures comprising the anti-PD-L1 antibody exhibit a longer survival time than the control cohorts. The results confirm that knotted ssRNA nanostructures comprising the anti-PD-L1 antibody are used to kill cancer cells in the presence of a cancer cell. Targeting efficiency may lower than expected, peptides and DNA nanostructures including aptamers and origami are further optimized to get better outcomes. Furthermore, since the knotted ssRNA nanostructures comprising the anti-PD-L1 antibody are easy to customize, different combinations of targeting molecules, spacers domains, and/or targeting agents are tested to obtain higher anti-tumor potency. In some embodiments, three or four targeting molecules are simultaneously added to the knotted ssRNA nanostructures, or multiple targeting molecules plus cytokines are added to the knotted ssRNA nanostructures, or multiple targeting molecules plus checkpoint blockade are added to the knotted ssRNA nanostructures.

ssRNA tetrahedron with 15 crossing numbers
(SEQ ID NO: 16):
GGGAGAGUUGCGAAGAAUCUAACACUAGUGUACAAGGGCUUCUC
CUUAUCAGAGAUCAUGAUGACAACAGGUGAAUGGAGGGUCUAACAUAAGA
GUAAUCCAAUGCUUCGAACUAACCUUCCCCUUGUGGUUAUCCUAGCCUAG
CAUAUACGUUCUGAGAAAACUUCCCUCUACUCACCAUCUGCAGCAAUACU
CUACUACAGGUCUCUUUUCACUCUAGGGGAUGCAGCUAACGACCUACGUG
UUAUGAAGGUCCUAGAUUUGAGUAGCUCCACUCUCGUGCAUGGGGUGGAC
GGUAACGCCCUUCUGGGUUUCUCAAAAGGGGUGUUGCCAGCUGAACUAUG
GCUUUGGCGUUUCUUUCGGAAGCGAAGCUGAUGCUUGCUGAGGCAGAUCC
ACUGAAAACCCUUGCCUCCAGACUCGAGCUUCGCGACAGCGUGGCCGGAU
CAUGCUCCCCUUAGUCAGAGUCUUACUGACGUAACAAAACAGGCAAAUGG
UCUCUGUCAAGAUGCCCCUUUGUGACCGUCGGGAUGUAUGUAACUUAUGC
GUGAUUUCACCGGCCAUACUCUGGUCGUCAUUCGCUGAUUGUAGGGAUGC
CAGCGGCCUCAACUACUAGUGGAUGUACAACCCCGGUAAUGCUCUGCCAU
ACCGGUAAGGGGACUAUAAGACCCACGUGUUACUCGCGCGUUAUAUGAUG
GCUCAUCAGCCAUGUUAUCCAAAAGUCCAGUUACCUCUGACUCCUCGCCG
UGCUGGGACUAUCUCGAGUCGCGAACUACAAUACGAGCAAUUCCGCAUCC
CAAAAGUCCAUAACUUGUAUUCGAUCCGCAUGCUAGAUGACAGUUUCUUG
UCGGCUGUGAACUGGUUGCGACGUGAGCUCUAGCGCCCUGUGUAACCUGA
AGAAGUAGUUGUGCUAUUGAACUCUUGUGCUAGAACCGAAUGACUCGAAG
UCUAGAAAUAGACUUCGACAUGUACUGUUCUAGCACAAGAGUUCAAUAGC
AAUCAGGUCUCUUCAGGUUACACAGGGCGCUAGAGAAAACUCACGUCGCC
UAGGCUACACAGCCGACAAGAGGAAGGUUUCUAGCAUGCGGAUGAUUACU
CAGUUAUGGACGGGAUGCGGAAUUGCUCGUAUUGUAGGGAUCAUGUCGAG
AUAGUCCCAGCACGGCGAGGUGGGCUUCGUAACUGGACGGAUAACAUGGC
UGAAUCCCCUACAUAUAACGCGCGACUGUAGUAUGGGUCUUAUAGUCAUG
GUGAGGGUAUGGCAGAGCAUAAAAUACCGGGGUUGUACAUCCACUGACCU
GAUAGGCCGCUGGCAUCCCUACAAUCAGAGUACAUGGACCAGAGUAUGGC
CAAAAGGUGAAAUCAGCGUUACCUUACAUACAUCCCGCGAGAGUGAAAGG
GGCAUCUUGUAGGACCUCAUUUGCCUGGUUACGUCAGUAAGAGAAGCCCA
AAGGGGAGCAUGAUCCGGCCACGCUCAUGAUCCGCUCGAGUCUGGAGGCA
AGGGCAGUGGAUCUGCCUCAUGUCAUCUCAGCUUCGCUUCCGAAAGAAAC
GCUGUACACUAAGUUCAGCUGGCAACACCCCGAGAAACCCAGAAGGCGCA
UAAGGUCCACCCCAUGCAACGGUCACGAGCUACUCAAAUCACAGAGACUC
AUAACACGUAGGUAAAACGUUAGCUGCUGAGCCAUGAGUGAAAAGAGACG
UAACACGGAGUAUUGCUGCAGCCCUUACCUAGAGGGAAGCUCAGAACGUA
UAUGAACCAGUUGGAUAACCACAAGGAACUGUCAAGUUCGAAGCAUUGCG
AAUACAUUAUGUUAGACCCUCAAAACAUUCACCUGUGCAAGCAAUGAUCU
CUGAUAAGGAGAAGCCCUCAAAGCCAUGUGUUAGAUUCUUCGCAACUCUC
CC
ssRNA tetrahedron without crossing numbers
(SEQ ID NO: 17):
GGGAGAUUACUCAUAAGGGCUGGCUUGGUUCACUAGGAGCUAGU
UGGGUAGCCCGUCACCAGUGCGUACAGCCCGUUCAUCCGCUUUGCGAUUG
CUCACACAACGCUUCGAGUUUACCCGUUCUGCGAUUGAUCGAAAGAUCAG
GACAUCGACGGGUGAACUCGAGUUGGGAAGUGAGCGAUCGCAAGCGAUCG
AAACGGAAAACUCGCUGCUUACCGUAUGAAUAGGAGGUACCUUCUGCCGG
UAGUCGUUCGUUCAGUAAGCUGAGCUCGAAAGAGCUGUAGUAGUUGAACG
GACGACUAACUUAGAUCGUAGAGACCGAGGCAUACGGUUCCUUGAAAAAG
GACGCAAUGACCUCGGUUUCUACGGUCUAAGUAAAUCAAUAUCACCACUA
CUACCUAUGCCACGAAAACCCAUUGCCGAGGAUCCACAAUGGUGCUCACG
CGUUUAUGUAGCAUUUUGAGCGGGAUCGGUUGAGAGAAAUCUCAUGGAGU
UACGCUCAAGAUGCUAGCACACGCCGAGCCUAUAGAGAUGGAUCCUGCUU
CGAAAGAAGCUCCUACGGUCUCUAUGGGCUCGGUGUGUGCCUAGCUCGUA
GCUCUAACUCCAAUCAUGGUGGAAAAUGAGUAGUCCAUCGCAGAGUAUUC
GGCCUGUGAGCGUUGUUACGGAUUUGCUGCAGCGGAUGGAGUUUAUGCGA
AAGCAUAGACUCUCGAUCGCGCAGCAGAUCCGUAUUCCCAACCACAGGUC
GAAUACCGAUGUCCGGACUGCUCAAAAAGAGCGGGGUUAGCAUGCGUUGC
CAUCUCAACAUCUCCGUACUGCACUCUACAUGACAAGUACGAGGGUAUCU
UGUUCGUGAGAUCGUUCAUGGUAGCACGCAGCUUCGGCUGAGGAGCGAUC
CACAACGCUCUAGAAAUAGAGCUGGUGACAUCGCUCUUCAGCCGCUCCUA
GGUGCUAUCAUGAACCCUUAUGAGAACAAAAAGUCGCGUGGGCCCCAAUG
CCUAGAGCUAAAUGCGAAAGGUGCAAGCUACGCACAGCGUCUGAUAAGGC
GAGUGAAAACUCGUCUUAGUUCGUCUUGUGCGUGGCUUGCCGCGAUUCCA
UUUAGUUCUAGGUCGUCUAUCCCAUGCGACAAAAAGAUAUCCUCCCUCUG
ACCAUGUAGCGUGCAGUGCGGAGAAGAGGUGUGAGACGCGCAUGCUGCGU
UGAAAAACGCUCGAAAACCGUCUCAUACCUCUCUCCGUGAUAUCAGUAGG
AUUCGUCAGAGGCGCAUGAAAAUGCGGUACUUGUGAAUCCUGCUGAUAUU
ACGGAGUGUUGAGGUGGCAAGUUUUCGAAACCUCGCUCCCACCGUGAUAC
CGAUCCGAGCUAUGAGCUAGCAUAAAUGCGUGAGUACCAUUGCCGUAGGA
CGGCGAUGGGUUGCCUCAGACGCAGCCCUAGUUAUCUACCUUUCGAUCCU
UGGCCACUUCAUUGGGGACUUCGAAAGAAGUAUAGACGAAAGUGGCUAAG
GAUGAAUCGCGAGAUAAUUAGGGCUAGACGAACGGCAAAAAACGUGGUAU
AGCAGCUUACGGUGAUGUUGAUUUCCGGCAGGAGGUACUUCCUAUUUCAU
UGCGAAGCGGCGAGAAAAGCUGUGCGCACGUUGUGGGGGCUACUCAACUA
GAAGCUGCUGAACCGAGCCAGCGAUCUCACGUAAUCUCCC

Although the foregoing specification and examples fully disclose and enable the embodiments of the present disclosure, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A knotted, self-assembled single-stranded nucleic acid (ssNA) nanostructure comprising a crossing number from 1 to 1,200 and comprising at least one paranemic cohesion crossover.

2. The knotted ssNA nanostructure of claim 1, wherein the nanostructure comprises a crossing number from 2 to 100 or 9 to 67.

3. (canceled)

4. The knotted ssNA nanostructure of claim 1, wherein the nucleic acid is DNA or RNA.

5. (canceled)

6. The knotted ssNA nanostructure of claim 1, comprising a nucleic acid (an NA) sequence of about 1000 to about 10,000 nucleotides in length, about 1800 to about 7500 nucleotides in length, or about 6500 to about 7500 nucleotides in length.

7-8. (canceled)

9. The knotted ssNA nanostructure of claim 1, wherein the nanostructure self-assembles into a 3-dimensional shape by means of paranemic cohesion into knots comprising four ssNA regions.

10-14. (canceled)

15. The knotted ssNA nanostructure of claim 1, comprising at least about 95 to 99% sequence identity to or comprises SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 or SEQ ID NO: 17.

16. The knotted ssNA nanostructure of claim 1, comprising DNA having a crossing number of 9 forming a square knotted DNA, comprising or consisting of a nucleic acid having the sequence of SEQ ID NO: 1.

17-18. (canceled)

19. The knotted ssNA nanostructure of claim 1, comprising DNA having a crossing number of 23 forming a 3-square knotted DNA before hierarchical design and comprising or consisting of a nucleic acid having the sequence of SEQ ID NO: 2 or SEQ ID NO: 3.

20-21. (canceled)

22. The knotted ssNA nanostructure of claim 1, comprising DNA having a crossing number of 57 forming a 9-square knotted DNA before hierarchical design and comprising or consisting of a nucleic acid having the sequence of SEQ ID NO: 4.

23-24. (canceled)

25. The knotted ssNA nanostructure of claim 1, comprising DNA having a crossing number of 57 forming a 9-square knotted DNA after hierarchical design and comprising or consisting of a nucleic acid having the sequence of SEQ ID NO: 5.

26-27. (canceled)

28. The knotted ssNA nanostructure of claim 1, comprising DNA having a crossing number of 67 forming a hexagonal knotted DNA and comprising or consisting of a nucleic acid having the sequence of SEQ ID NO: 6.

29-30. (canceled)

31. The knotted ssNA nanostructure of claim 1, comprising RNA having a crossing number of 9 forming a square knotted RNA and comprising or consisting of a nucleic acid having the sequence of SEQ ID NO: 7.

32-33. (canceled)

34. The knotted ssNA nanostructure of claim 1, comprising DNA having a crossing number of 15 forming a tetrahedron and comprising or consisting of a nucleic acid having the sequence of SEQ ID NO: 8 or SEQ ID NO: 17.

35-36. (canceled)

37. The knotted ssNA nanostructure of claim 1, comprising DNA having a crossing number of 20 forming a pyramid and comprising or consisting of a nucleic acid having the sequence of SEQ ID NO: 9.

38-39. (canceled)

40. The knotted ssNA nanostructure of claim 1, comprising DNA having a crossing number of 22 forming a triangular prism and comprising or consisting of a nucleic acid having the sequence of SEQ ID NO: 10.

41-42. (canceled)

43. The knotted ssNA nanostructure of claim 1, comprising DNA having a crossing number of 25 forming a pentagonal pyramid and comprising or consisting of a nucleic acid having the sequence of SEQ ID NO: 11.

44-45. (canceled)

46. The knotted ssNA nanostructure of claim 1, further comprising a topological control strand comprising or consisting of a sequence selected from a nucleic acid having the sequence of SEQ ID NO: 12, SEQ ID NO: 13, SEQ ID NO: 14 or SEQ ID NO: 15.

47. (canceled)

48. The knotted ssNA nanostructure of claim 1, further comprising a 5′-end and a 3′-end, wherein the 5′-end and/or the 3′-end further comprises 1-5 nucleotide terminal extensions and the terminal extensions hybridize to form a single strand of nucleic acid.

49. (canceled)

50. The knotted ssNA nanostructure of claim 1, further comprising at least one diagnostic agent or therapeutic agent operably linked to said nanostructure.

51-53. (canceled)

54. A pharmaceutical composition comprising the knotted ssNA nanostructure of claim 1, and a pharmaceutically acceptable carrier, and a chemotherapeutic drug.

55-56. (canceled)

57. A method of treating a disease or disorder in a subject, comprising administering to the subject a therapeutically effective amount of the composition of claim 54.

58-72. (canceled)
